(19) United States
(12) Patent Application Publication
(10) Pub. No.: US 2016/0009767 A9
(43) Pub. Date: Jan. 14, 2016

(54) ORAL ADMINISTRATION

(71) Applicant: AFFIBODY AB, Solna (SE)

(72) Inventors: David Bejker, Huddinge (SE); Caroline Ekblad, Saltsjo-Boo (SE)

(21) Appl. No.: 14/385,618

(22) PCT Filed: Mar. 15, 2013
(86) PCT No.: PCT/EP2013/055441

§ 371 Date: Sep. 16, 2014

Related U.S. Application Data
(60) Provisional application No. 61/616,490, filed on Mar. 28, 2012.

(30) Foreign Application Priority Data
Mar. 28, 2012 (EP) 12161808.6

(51) Int. Cl. C07K 14/315; C07K 14/31; C07K 14/195; C07K 14/00; A61K 9/00

(52) U.S. Cl. CPC C07K 14/315; C07K 14/00; A61K 9/0053; C07K 14/195; C07K 14/31; C07K 2319/31

(57) ABSTRACT

The present invention is within the field of administration of biopharmaceuticals. More specifically, the invention provides for oral administration of a compound comprising a moiety which confers a desired therapeutic activity; and a polypeptide moiety which binds to albumin.

### FIELD OF THE INVENTION

The present invention is within the field of administration of biopharmaceuticals. More specifically, the invention provides for oral administration of a compound comprising a moiety which confers a desired therapeutic activity; and a polypeptide moiety which binds to albumin.

### BACKGROUND

### Oral Delivery of Protein Therapeutics

The majority of protein and peptide therapeutics currently on the market are administered by the parenteral route, i.e. without passing the gastrointestinal tract, such as by intravenous, intramuscular or subcutaneous injections. Intravenous administration directly into the systemic circulation provides 100% bioavailability and fast onset of drug action. However, the instant high concentration of the drug in the blood increases the risk of side effects. Furthermore, administration by any injection method is associated with low patient compliance due to the pain and discomfort. Self-administration is often not possible and hence treatment has to be carried out in the clinic. The latter becomes a particular problem if the half-life of the drug is short, and frequent, repeated administrations are required to maintain adequate levels of therapeutic action. Clinical treatment, and in some cases necessary hospitalization of the patient, also implies increased costs for society. Simplified administration is thus a major driving force for development of drugs intended for alternative delivery routes such as oral, intranasal, pulmonary, transdermal or rectal, each of which is associated with specific advantages and limitations. Oral administration remains one of the most convenient administration routes, in particular for the treatment of pediatric patients. Furthermore, oral formulations do not require production under sterile conditions, which reduces the manufacturing costs per unit of drug (Salama et al, Adv Drug Deliv Rev. 58:15-28, 2006). For some protein therapeutics, the oral delivery route may even be more physiological, as has been suggested for insulin (Hoffman and Ziv, Clin Pharmacokinet. 33:285-301, 1997).

Oral delivery of conventional low molecular weight drugs has been well established in practice. However, oral delivery of larger, less stable and often polar, peptide and protein therapeutics faces other challenges including that the drug must 1) be resistant to the acidic environment of the stomach 2) be resistant to enzymatic degradation in the gastrointestinal tract and 3) be able to cross the intestinal epithelium and reach into the circulation. Different approaches have been attempted to address these challenges either by modifying the protein itself, or by optimizing the formulation or drug carrier system.

### Factors Influencing Oral Bioavailability

The bioavailability of a protein therapeutic administered orally depends on the physiological properties of the protein, such as molecular weight, amino acid sequence, hydrophobicity, isoelectric point (pI), solubility and pH stability, as well as on the biological barriers encountered in the gastrointestinal tract, i.e. the proteolytic environment and the generally poor absorption of large molecules through the intestinal wall.

The physiochemical environment of the gastrointestinal tract varies depending on the feeding status of the individual. Factors that vary between the fasted and fed stages include pH, the composition of gastrointestinal fluids and the volume of the stomach. In humans, the pH of the stomach is around 1-2 in the fed state whereas it rises to 3-7 in the fasted state. The pH varies throughout the small intestine, but averages around pH 5 and 6.5 in the fed and fasted state, respectively (Klein, AAPS J. 12:397-406, 2010). The differences in pH affect the level of activity of proteolytic enzymes, which are each associated with a specific pH optimum. Pepsin, the predominant protease in the stomach, has optimal activity around pH 2, whereas trypsin and chymotrypsin of the intestine has optimal activity around pH 8. Furthermore, gastric emptying is a rate-limiting step. Food, in particular fatty food, slows gastric emptying and hence the rate of drug absorption (Singh, Clin Pharmacokinet. 37:213-55, 1999), and thus prolongs the time for which the drug is exposed to proteolytic enzymes. Therefore, the bioavailability of the drug can be affected if the drug is taken during or in between meals, with or without a significant of volume liquid, or different types of liquid.

Poor absorption through the intestinal wall remains the main factor limiting the bioavailability of orally delivered protein therapeutics. Drugs taken orally have, as with any nutrient, two options to cross the intestinal wall; by using either the transcellular pathway, which involves passage across cells, or the paracellular pathway, which involves passage between adjacent cells via tight junctions. Small molecules with a molecular weight less than 500 Da can cross using either pathway (Müller, Curr Issues Mol Biol. 13:13-24, 2011). The ability of drugs with a larger molecular weight to cross the intestinal wall depends on the physiochemical properties of the drug, such as charge, lipophilicity and hydrophilicity. For lipophilic drugs, the transcellular route dominates, whereas hydrophilic drugs can cross by the paracellular route (Salama et al, 2006, supra). However, the dimension of the paracellular space is between 10 and 30-50 Å and it has been suggested that the paracellular transport is generally limited to molecules with a radius less than 15 Å (˜3.5 kDa) (Rubas et al, J Pharm Sci. 85:165-9, 1996). As for the transcellular pathway, small molecular weight substances readily cross by passive diffusion. However, larger molecular weight substances are confined to active processes requiring energy expenditure, such as pinocytocis (nonspecific “cell drinking”) or transcytosis (receptor-mediated transport).

Finally, bioavailability is also influenced by interpatient variability, including age (drugs are generally metabolized more slowly in fetal, neonatal and geriatric populations), health of the gastrointestinal tract, and general disease state (e.g. hepatic insufficiency, poor renal function), as well as intrapatient variability i.e. variability in the same patient over time.

Increasing the bioavailability of orally administered proteins and peptides is crucial for enabling delivery of a therapeutically effective dose, reducing the manufacturing costs and to a lesser extent having to account for interpatient and intrapatient variability. Strategies to improve the oral bioavailability of protein therapeutics have ranged from changing the physiochemical properties such as hydrophobicity, charge, pH stability and solubility; inclusion of protease inhibitors or absorbance enhancers in the drug formulation; and use of formulation vehicles such as emulsions, liposomes, microspheres or nanoparticles (reviewed in Park et al, Reactive and Functional Polymers, 71:280-287, 2011).

### Prolonging the In Vivo Half-Life of Proteins

Considering the relatively low bioavailability of orally administered peptide and protein drugs, it becomes relevant to maintain a long in vivo plasma half-life of the fraction that manages to cross the intestinal epithelial membrane in a biologically active form. Several strategies for preventing rapid renal clearance have been described in the literature, and are known to the person skilled in the art. These strategies include fusion, conjugation or association with albumin, antibodies or fragments thereof, or conjugation to one or several polyethylene glycol (PEG) derivatives. PEGylation has also been reported to promote the absorption through the mucosa, stabilize peptide drugs and prevent degradation by proteases (Meibohm, Pharmacokinetics and Pharmacodynamics of Biotech Drugs, Wiley-VCH, 2006). In vivo post-administration association with molecules exhibiting long half-life may be favored over direct fusion or conjugation to the same prior to administration, so as to retain a small size and prevent proteolytic degradation of the part of the molecule exhibiting the long half-life.

Association with Serum Albumin for Increasing the In Vivo Half-Life of Proteins

Serum albumin is the most abundant protein in mammalian sera (35-50 g/l, i.e. 0.53-0.75 mM, in humans) and several strategies to covalently couple a peptide or protein to carrier molecule that will allow in vivo association to serum albumin have been described e.g. in WO91/01743, in WO01/45746 and in Dennis et al (J Biol Chem 277:35035-43, 2002). The first document describes inter alia the use of albumin binding peptides or proteins derived from streptococcal protein G (SpG) for increasing the half-life of other proteins. The idea is to fuse the bacterially derived, albumin binding peptide/protein to a therapeutically interesting peptide/protein, which has been shown to have a rapid elimination from blood. The generated fusion protein binds to serum albumin in vivo, and benefits from its longer half-life, which increases the net half-life of the fused therapeutically interesting peptide/protein. WO01/45746 and Dennis et al relate to the same concept, but here, the authors utilize relatively short peptides to bind serum albumin. The peptides were selected from a phage displayed peptide library. The US patent application published as US2004/0001827 (Dennis) also discloses the use of constructs comprising peptide ligands, again identified by phage display technology, which bind to serum albumin and which are conjugated to bioactive compounds for tumor targeting.

### Albumin Binding Domains of Bacterial Receptor Proteins

Streptococcal protein G (SpG) is a bi-functional receptor present on the surface of certain strains of streptococci and is capable of binding to both IgG and serum albumin (Björck et al, Mol Immunol 24:1113, 1987). The structure is highly repetitive with several structurally and functionally different domains (Guss et al, EMBO J 5:1567, 1986), more precisely three Ig-binding domains and three serum albumin binding domains (Olsson et al, Eur J Biochem 168:319, 1987). The structure of one of the three serum albumin binding domains in SpG has been determined, showing a three-helix bundle fold (Kraulis et al, FEBS Lett 378:190, 1996; Johansson et al, J. Biol. Chem. 277:8114-20, 2002). A 46 amino acid motif was defined as ABD (albumin binding domain) and has subsequently also been designated G148-GA3 (GA for protein G-related albumin binding.

Other bacterial albumin binding domains than the ones in protein G have also been identified, some of which are structurally similar to the ones of protein G. Examples of proteins containing such albumin binding domains are the PAB, PPL, MAG and ZAG proteins (Rozak et al, Biochemistry 45:3263-3271, 2006). Structural and functional studies of such albumin binding domains have been carried out and reported e.g. by Johansson and co-workers (Johansson et al, J Mol Biol 266:859-865, 1997).

In addition to the three-helix bundle proteins described above, there are also other unrelated bacterial proteins that bind albumin. For example, the family of streptococcal proteins designated the “M proteins” comprises members that bind albumin (see e.g. Table 2 in Navarre & Schneewind, MMBR 63:174-229, 1999). Non-limiting examples are proteins M1/Emm1, M3/Emm3, M12/Emm12, EmmL55/Emm55, Emm49/EmmL49, and H.

### Engineered ABD Variants

Rozak et al have reported the creation of artificial variants of G148-GA3, which were selected and studied with regard to different species specificity and stability (Rozak et al, 2006, supra), whereas Jonsson et al developed artificial variants of G148-GA3 having very much improved affinity for human serum albumin (Jonsson et al, Prot Eng Des Sel 21:515-27, 2008; WO2009/016043).

A few T- and B-cell epitopes have been experimentally identified within the albumin binding region of streptococcal protein G strain 148 (G148) (Goetsch et al, Clin Diagn Lab Immunol 10:125-32, 2003), making the albumin binding domain G148 as such less suitable for use in pharmaceutical compositions for human administration. To reduce the immune stimulatory properties, new ABD variants with fewer potential B- and T-cell epitopes, but with retained high albumin binding capacity, were developed as described in WO2012/004384.

As is evident from the background description above, there remains a need for therapeutically effective biopharmaceuticals which can be administered via the oral route.

### DESCRIPTION

The different aspects of the present invention address this need through enabling the oral administration of molecules as defined further below. Thus, the invention provides such molecules for use in treatment via oral administration; pharmaceutical compositions which comprise such molecules and are formulated to be suited to oral administration; and treatment methods in which such molecules or pharmaceutical compositions are administered orally to a subject in need of such treatment.

### I Compound for Use

In a first aspect, the present invention provides a compound for use in treatment via oral administration, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment.

The compound as defined above comprises at least the two moieties (I) and (II), which may for example be connected by covalent coupling using known organic chemistry methods, or, if one or both moieties are polypeptides, be expressed as one or more fusion polypeptides in a system for recombinant expression of polypeptides, or joined in any other fashion, directly or mediated by a linker comprising a number of amino acids. For discussions concerning the coupling of albumin binding moieties to other moieties, for example in order to provide a compound as defined above, see for example PCT publications WO2010/054699 and WO2012/004384, incorporated herein by reference.

### Moiety (I) Conferring a Desired Therapeutic Activity

In one embodiment of the present invention, the part of the compound designated moiety (I) comprises a component selected from the group consisting of human endogenous enzymes, hormones, growth factors, chemokines, cytokines, blood clotting and complement factors, innate immune defense and regulatory peptides, for example selected from the group consisting of insulin, insulin analogs, IL-2, IL-5, GLP-1, BNP, IL 1-RA, KGF, Stemgen®, GH, G-CSF, CTLA-4, myostatin, Factor VII, Factor VIII and Factor IX, and derivatives of anyone thereof.

In another embodiment, moiety (I) comprises a non-human biologically active protein, selected from the group consisting of modulins, bacterial toxins, hormones (excluding exendins), innate immune defense and regulatory peptides, enzymes and activating proteins.

In yet another embodiment, moiety (I) comprises a binding polypeptide capable of selective interaction with a target molecule. Such a binding polypeptide may for example be selected from the group consisting of antibodies and fragments and domains thereof substantially retaining antibody binding activity; microbodies, maxybodies, avimers and other small disulfide-bonded proteins; and binding proteins derived from a scaffold selected from the group consisting of staphylococcal protein A and domains thereof, other three helix domains, lipocalins, ankyrin repeat domains, cellulose binding domains, γ crystallines, green fluorescent protein, human cytotoxic T lymphocyte-associated antigen 4, protease inhibitors such as Kunitz domains, PDZ domains, SH3 domains, peptide aptamers, staphylococcal nuclease, tendamistats, fibronectin type III domain, transferrin, zinc fingers and conotoxins.

In some examples of such an embodiment, the binding polypeptide comprises a variant of protein Z, in turn derived from domain B of staphylococcal protein A and described in Nilsson B et al, Protein Engineering 1:107-133, 1987. Such variants, having affinity for a number of different targets, have been selected from libraries and engineered further as described in numerous prior publications, for example but not limited to WO95/19374; Nord et al, Nat Biotech (1997) 15:772-777; and WO2009/080811, all incorporated herein by reference. In this embodiment of a compound for use according to the present invention, the variant of protein Z which corresponds to moiety (I) comprises a scaffold amino acid sequence selected from SEQ ID NO:719, SEQ ID NO:720 and SEQ ID NO:721, wherein X denotes any amino acid residue. As described in the article and PCT publications referred to above, the amino acid positions comprising an X are all involved in the binding function of the protein Z variant, and will vary depending on what target the Z variant is designed to bind. Preferably in these embodiments, the scaffold amino acid sequence of moiety (I) comprises SEQ ID NO:719 or SEQ ID NO:720.

In embodiments of the present invention wherein moiety (I) comprises a binding polypeptide capable of selective interaction with a target molecule, said target molecule may be selected from the group consisting of tumor-related or other cell surface related antigens, such as CD14, CD19, CD20, CD22, CD30, CD33, CD37, CD40, CD52, CD56, CD70, CD138, cMet, HER1, HER2, HER3, HER4, CAIX, CEA, IL-2 receptor, IGF1R, VEGFR2, MUC1, PDGFR-beta, PSMA, TAG-72, FOLR1, mesothelin, CA6, GPNMB, integrins and ephA2; cytokines such as TNF-α, IL-1α, IL-1β, IL-1Ra, IL-5, IL-6, IL-13, IL-17A, IL-18, IL-23, IL-36, G-CSF, GM-CSF, and their receptors; chemokines such as IL-8, CCL-2 and CCL11, and their receptors; complement factors such as C3 and factor D; growth factors such as HGF and myostatin; hormones such as GH, insulin and somatostatin; peptides such as Aβ peptide of Alzheimer's disease; other disease-associated amyloid peptides; hypersensitivity mediators such as histamine and IgE; blood clotting factors, such as von Willebrand factor; and toxins, such as bacterial toxins and snake venoms.

In an alternative embodiment, moiety (I) comprises a non-proteinaceous component having a therapeutic activity. Examples of particular interest are cytotoxic agents and anti-inflammatory agents, since albumin has been shown to accumulate in tumor tissues and at sites of inflammation (Kratz and Beyer, Drug Delivery 5: 281-99, 1998; Wunder et al, J. Immunol. 170: 4793-801, 2003). This, in turn, provides a rationale for oral delivery of such compounds together with the albumin binding moiety for targeting and accumulation at relevant tumor tissues or inflammation sites. Non-limiting examples of cytotoxic agents are calicheamycin, auristatin, doxorubicin, maytansinoid, taxane, ecteinascidin, geldanamycin, methotrexate, camptothecin, cyclophosphamide, cyclosporine and their derivatives, and combinations thereof. Non-limiting examples of anti-inflammatory agents are non-steroidal anti-inflammatory drugs (NSAIDs), cytokine suppressive anti-inflammatory drugs (CSAIDs), corticosteroids, methotrexate, prednisone, cyclosporine, morroniside cinnamic acid, leflunomide and their derivatives, and combinations thereof.

In such embodiments, the non-proteinaceous moiety (I) and albumin binding moiety (II) may be non-covalently associated, but it is currently preferred that they be covalently coupled together.

Conjugation of a non-proteinaceous moiety (I) to an albumin binding moiety (II) may increase the solubility, and thereby the bioavailability, of poorly soluble compounds otherwise not suitable for oral administration.

Moiety (II) which Binds to Albumin

As defined herein, the compound for use in treatment via oral administration comprises an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof.

As explained in the background section and in the articles by Rozak et al and Johansson et al cited therein, many of the above albumin binding proteins comprise albumin binding domains denoted GA domains. In one embodiment of the present invention, moiety (II) of the compound comprises a naturally occurring GA domain or a derivative thereof. Specific examples of useful such GA domains are domain GA1, domain GA2 and domain GA3 of protein G from Streptococcus strain G148, and derivatives thereof. In one specific embodiment, moiety (II) comprises domain GA3 of protein G from Streptococcus strain G148. This albumin binding domain is also frequently denoted “ABD” or “ABDwt” in the literature, and has the amino acid sequence of SEQ ID NO:515 in the appended listing. In another embodiment, moiety (II) comprises a derivative of domain GA3 of protein G from Streptococcus strain G148. Several such derivatives have been developed, for example as described in the abovementioned WO2009/016043 and WO2012/004384, incorporated herein by reference.

### Moiety (II) Comprising an ABD Derivative as Disclosed in WO2009/016043

Thus, with reference to WO2009/016043, moiety (II) of the compound for use in treatment via oral administration according to the invention may comprise an albumin binding motif, which motif consists of the amino acid sequence:

GVSDX₅YKX₈X₉I X₁₁X₁₂AX₁₄TVEGVX₂₀ ALX₂₃X₂₄X₂₅I

wherein, independently of each other,
X₅ is selected from Y and F;
X₈ is selected from N, R and S;
X₉ is selected from V, I, L, M, F and Y;
X₁₁ is selected from N, S, E and D;
X₁₂ is selected from R, K and N;
X₁₄ is selected from K and R;
X₂₀ is selected from D, N, Q, E, H, S, R and K;
X₂₃ is selected from K, I and T;
X₂₄ is selected from A, S, T, G, H, L and D; and
X₂₅ is selected from H, E and D.

The above definition of a class of sequence related, albumin binding polypeptides for use in moiety (II) in the compound is based on a statistical analysis of a large number of albumin binding polypeptides identified and characterized as detailed in the experimental section of WO2009/016043. Briefly, the variants were selected from a large pool of random variants of the parent polypeptide sequence of ABDwt (SEQ ID NO:515), said selection being based on an interaction with albumin in e.g. phage display or other selection experiments. The identified albumin binding motif, or “ABM”, corresponds to the albumin binding region of the parent scaffold, which region constitutes two alpha helices within a three-helical bundle protein domain. While the original amino acid residues of the two ABM helices in the parent scaffold already constitute a binding surface for interaction with albumin, that binding surface is modified by the substitutions according to the invention to provide an alternative albumin binding ability.

In one embodiment, X₅ is Y.

In one embodiment, X₈ is selected from N and R, and may in particular be R.

In one embodiment, X₉ is L.

In one embodiment, X₁₁ is selected from N and S, and may in particular be N.

In one embodiment, X₁₂ is selected from R and K, such as X₁₂ being R or X₁₂ being K.

In one embodiment, X₁₄ is K.

In one embodiment, X₂₀ is selected from D, N, Q, E, H, S and R, and may in particular be E.

In one embodiment, X₂₃ is selected from K and I, and may in particular be K.

In one embodiment, X₂₄ is selected from A, S, T, G, H and L.

In a more specific embodiment, X₂₄ is L.

In an even more specific embodiment, X₂₃X₂₄ is KL.

In another even more specific embodiment, X₂₃X₂₄ is TL.

In one embodiment, X₂₄ is selected from A, S, T, G and H.

In a more specific embodiment, X₂₄ is selected from A, S, T, G and H and X₂₃ is I.

In one embodiment, X₂₅ is H.

As described in detail in the experimental section of WO2009/016043, the selection of albumin binding variants led to the identification of a substantial amount of individual albumin binding motif (ABM) sequences. These sequences constitute individual embodiments of the ABM sequence in the definition of an albumin binding amino acid sequence as moiety (II) in the context of the present invention. The sequences of individual albumin binding motifs are presented in FIG. 1 as SEQ ID NO:1-257. In certain embodiments, the ABM consists of an amino acid sequence selected from SEQ ID NO:1-257. In a more specific embodiment, the ABM sequence is selected from SEQ ID NO:2, SEQ ID NO:3, SEQ ID NO:9, SEQ ID NO:15, SEQ ID NO:25, SEQ ID NO:27, SEQ ID NO:46, SEQ ID NO:49, SEQ ID NO:53, SEQ ID NO:54, SEQ ID NO:55, SEQ ID NO:155, SEQ ID NO:239, SEQ ID NO:240, SEQ ID NO:241, SEQ ID NO:242, SEQ ID NO:243, SEQ ID NO:244 and SEQ ID NO:245. In yet more specific embodiments, the ABM sequence is selected from SEQ ID NO:3, SEQ ID NO:53 and SEQ ID NO:239.

In embodiments of the albumin binding moiety (II), the ABM may form part of a three-helix bundle protein domain. For example, the ABM may essentially constitute or form part of two alpha helices with an interconnecting loop, within said three-helix bundle protein domain.

In particular embodiments, such a three-helix bundle protein domain is selected from the group consisting of three-helix domains of bacterial receptor proteins. Non-limiting examples of such bacterial receptor proteins are selected from the group consisting of albumin binding receptor proteins from species of Streptococcus, Peptostreptococcus and Finegoldia, such as for example selected from the group consisting of proteins G, MAG, ZAG, PPL and PAB. In a specific embodiment of the invention, the ABM forms part of protein G, such as for example protein G from Streptococcus strain G148. In different variants of this embodiment, the three-helix bundle protein domain of which the ABM forms a part is selected from the group consisting of domain GA1, domain GA2 and domain GA3 of protein G from Streptococcus strain G148, in particular domain GA3.

In alternative embodiments, the ABM forms part of one or more of the five three-helix domains of the bacterial receptor protein A from Staphylococcus aureus; i.e. the three-helix bundle protein domain is selected from the group consisting of protein A domains A, B, C, D and E. In other similar embodiments, the ABM forms part of protein Z, derived from domain B of protein A from Staphylococcus aureus.

In embodiments wherein the ABM “forms part of” a three-helix bundle protein domain, this is understood to mean that the sequence of the ABM is “inserted” into or “grafted” onto the sequence of the naturally occurring (or otherwise original) three-helix bundle domain, such that the ABM replaces a similar structural motif in the original domain. For example, without wishing to be bound by theory, the ABM is thought to constitute two of the three helices of a three-helix bundle, and can therefore replace such a two-helix motif within any three-helix bundle. As the skilled person will realize, the replacement of two helices of the three-helix bundle domain by the two ABM helices has to be performed so as not to affect the basic structure of the polypeptide. That is, the overall folding of the Ca backbone of the polypeptide according to this embodiment will be substantially the same as that of the three-helix bundle protein domain of which it forms a part, e.g. having the same elements of secondary structure in the same order etc. Thus, an ABM according to the invention “forms part” of a three-helix bundle domain if the polypeptide according to this embodiment of the invention has the same fold as the original domain, implying that the basic structural properties are shared, those properties e.g. resulting in similar CD spectra. The skilled person is aware of other parameters that are relevant.

In one embodiment, the albumin binding polypeptide is a three-helix bundle protein domain, which comprises the albumin binding motif as defined above and additional sequences making up the remainder of the three-helix configuration. Thus, in this embodiment, moiety (II) comprises an albumin binding domain having the amino acid sequence:

LAEAKXₐX_{b}AX_{c}X_{d} ELXₑKY-[ABM]-LAALP

wherein
[ABM] is an albumin binding motif as defined above in this section,
and, independently of each other,
Xₐ is selected from V and E;
X_{b} is selected from L, E and D;
X_{c} is selected from N, L and I;
X_{d} is selected from R and K; and
Xₑ is selected from D and K.

In one embodiment, Xₐ is V.

In one embodiment, X_{b} is L.

In one embodiment, X_{c} is N.

In one embodiment, X_{d} is R.

In one embodiment, Xₑ is D.

Again, as described in detail in the experimental section of WO2009/016043, the selection and sequencing of a number of albumin binding variants led to the identification of individual albumin binding domain sequences. These sequences constitute individual embodiments of the albumin binding domain comprised in moiety (II) in the compound for use in treatment by oral administration of the present invention. The sequences of these individual albumin binding domains are presented in FIG. 1 and as SEQ ID NO:258-514. Also encompassed by the definition herein is an albumin binding domain having an amino acid sequence with 85% or greater identity to a sequence selected from SEQ ID NO:258-514. In particular embodiments, the sequence of the albumin binding domain is selected from SEQ ID NO:259, SEQ ID NO:260, SEQ ID NO:266, SEQ ID NO:272, SEQ ID NO:282, SEQ ID NO:284, SEQ ID NO:303, SEQ ID NO:306, SEQ ID NO:310, SEQ ID NO:311, SEQ ID NO:312, SEQ ID NO:412, SEQ ID NO:496, SEQ ID NO:497, SEQ ID NO:498, SEQ ID NO:499, SEQ ID NO:500, SEQ ID NO:501 and SEQ ID NO:502 and sequences having 85% or greater identity thereto. In more specific embodiments of this aspect of the invention, the sequence of the albumin binding polypeptide is selected from SEQ ID NO:260, SEQ ID NO:310 and SEQ ID NO:496 and sequences having 85% or greater identity thereto.

### Moiety (II) Comprising an ABD Derivative as Disclosed in WO2012/004384

With reference instead to WO2012/004384, moiety (II) of the compound for use in treatment via oral administration according to the invention may instead comprise an albumin binding domain, which in turn comprises an amino acid sequence selected from

i) LAX₃AKX₆X₇ANX₁₀ ELDX₁₄YGVSDF YKRLIX₂₆KAKT
VEGVEALKX₃₉X₄₀ ILX₄₃X₄₄LP

wherein independently of each other
X₃ is selected from E, S, Q and C;
X₆ is selected from E, S and C;
X₇ is selected from A and S;
X₁₀ is selected from A, S and R;
X₁₄ is selected from A, S, C and K;
X₂₆ is selected from D and E;
X₃₉ is selected from D and E;
X₄₀ is selected from A and E;
X₄₃ is selected from A and K;
X₄₄ is selected from A, S and E;
L in position 45 is present or absent; and
P in position 46 is present or absent;
and
ii) an amino acid sequence which has at least 95% identity to the sequence defined in i).

The albumin binding domains according to this definition exhibit a set of characteristics, which, for example, make them suitable for use as fusion or conjugate partners for therapeutic molecules for human administration. The advantages of this class of albumin binding domains are explained in detail in WO2012/004384.

In one embodiment, X₆ is E.

In another embodiment, X₃ is S.

In another embodiment, X₃ is E.

In another embodiment, X₇ is A.

In another embodiment, X₁₄ is S.

In another embodiment, X₁₄ is C.

In another embodiment, X₁₀ is A.

In another embodiment, X₁₀ is S.

In another embodiment, X₂₆ is D.

In another embodiment, X₂₆ is E.

In another embodiment, X₃₉ is D.

In another embodiment, X₃₉ is E.

In another embodiment, X₄₀ is A.

In another embodiment, X₄₃ is A.

In another embodiment, X₄₄ is A.

In another embodiment, X₄₄ is S.

In another embodiment, the L residue in position 45 is present.

In another embodiment, the P residue in position 46 is present.

In another embodiment, the P residue in position 46 is absent.

In another embodiment, the albumin binding domain according to the definition in this section is subject to the proviso that X₇ is neither L, E nor D.

The albumin binding domain according to the definition in this section for use in moiety (II) may be prepared for conjugation with a suitable conjugation partner as described in detail in WO2012/004384.

In one embodiment, the amino acid sequence of the albumin binding domain of moiety (II) is selected from any one of SEQ ID NO:516-659 and SEQ ID NO:679-718, such as selected from any one of SEQ ID NO:516-659. More specifically, the amino acid sequence is selected from SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557, SEQ ID NO:564-565, SEQ ID NO:679-685 and SEQ ID NO:707-718. Thus, the amino acid sequence may be selected from SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557 and SEQ ID NO:564-565.

In one embodiment, the albumin binding domain according to this definition further comprises one or more additional amino acid residues positioned at the N- and/or the C-terminal of the sequence defined in i). These additional amino acid residues may play a role in enhancing the binding of albumin by the domain, and improving the conformational stability of the folded albumin binding domain, but may equally well serve other purposes, related for example to one or more of production, purification, stabilization in vivo or in vitro, coupling, labeling or detection of the polypeptide, as well as any combination thereof. Such additional amino acid residues may comprise one or more amino acid residue(s) added for purposes of chemical coupling, e.g. to the moiety (I) conferring a therapeutic effect; to a chromatographic resin to obtain an affinity matrix or to a chelating moiety for complexing with a radiometal.

The amino acids directly preceding or following the alpha helix at the N- or C-terminus of the amino acid sequence i) may thus in one embodiment affect the conformational stability. One example of an amino acid residue which may contribute to improved conformational stability is a serine residue positioned at the N-terminal of the amino acid sequence i) as defined above. The N-terminal serine residue may in some cases form a canonical S-X-X-E capping box, by involving hydrogen bonding between the gamma oxygen of the serine side chain and the polypeptide backbone NH of the glutamic acid residue. This N-terminal capping may contribute to stabilization of the first alpha helix of the three helix domain constituting the albumin binding domain according to this definition.

Thus, in one embodiment, the additional amino acids comprise at least one serine residue at the N-terminal of the domain. The amino acid sequence is in other words preceded by one or more serine residue(s). In another embodiment, the additional amino acids comprise a glycine residue at the N-terminal of the domain. It is understood that the amino acid sequence i) may be preceded by one, two, three, four or any suitable number of amino acid residues. Thus, the amino acid sequence may be preceded by a single serine residue, a single glycine residue or a combination of the two, such as a glycine-serine (GS) combination or a glycine-serine-serine (GSS) combination. Examples of albumin binding domains comprising additional amino residues at the N-terminal are set out in SEQ ID NO:660-678, such as in SEQ ID NO:660-663 and SEQ ID NO:677-678. In yet another embodiment, the additional amino acid residues comprise a glutamic acid at the N-terminal as defined by the sequence i).

Similarly, C-terminal capping may be exploited to improve stability of the third alpha helix of the three helix domain constituting the albumin binding domain. A proline residue, when present at the C-terminal of the amino acid sequence defined in i), may at least partly function as a capping residue. In such a case, a lysine residue following the proline residue at the C-terminal may contribute to further stabilization of the third helix of the albumin binding domain, by hydrogen bonding between the epsilon amino group of the lysine residue and the carbonyl groups of the amino acids located two and three residues before the lysine in the polypeptide backbone, e.g., when both L45 and P46 are present, the carbonyl groups of the leucine and alanine residues of the amino acid sequence defined in i). Thus, in one embodiment, the additional amino acids comprise a lysine residue at the C-terminal of the domain.

The additional amino acids may be related to the production of the albumin binding domain. In particular, when an albumin binding domain according to an embodiment in which P46 is present is produced by chemical peptide synthesis, one or more optional amino acid residues following the C-terminal proline may provide advantages. Such additional amino acid residues may for example prevent formation of undesired substances, such as diketopiperazine at the dipeptide stage of the synthesis. One example of such an amino acid residue is glycine. Thus, in one embodiment, the additional amino acids comprise a glycine residue at the C-terminal of the domain, directly following the proline residue or following an additional lysine and/or glycine residue as accounted for above. Alternatively, polypeptide production may benefit from amidation of the C-terminal proline residue of the amino acid sequence i), when present. In this case, the C-terminal proline comprises an additional amine group at the carboxyl carbon. In one embodiment of the domains described in this section, particularly those ending at their C-terminus with proline or other amino acid known to racemize during peptide synthesis, the above-mentioned addition of a glycine to the C-terminus or amidation of the proline, when present, can also counter potential problems with racemization of the C-terminal amino acid residue. If the domain, amidated in this way, is intended to be produced by recombinant means, rather than by chemical synthesis, amidation of the C-terminal amino acid can be performed by several methods known in the art, e.g. through the use of amidating PAM enzyme.

Examples of albumin binding domains comprising additional amino acid residues at the C-terminal are set out in SEQ ID NO:660-667, such as in SEQ ID NO:663-665. The skilled person is aware of methods for accomplishing C-terminal modification, such as by different types of pre-made matrices for peptide synthesis.

In another embodiment, the additional amino acid residues comprise a cysteine residue at the N- and/or C-terminal of the domain. Such a cysteine residue may directly precede and/or follow the amino acid sequence as defined in i) or may precede and/or follow any other additional amino acid residues as described above. Examples of albumin binding domains comprising a cysteine residue at the N- and/or C-terminal of the polypeptide chain are set out in SEQ ID NO:664-665 (C-terminal) and SEQ ID NO:666-667 (N-terminal). By the addition of a cysteine residue to the polypeptide chain, a thiol group for site directed conjugation of the albumin binding domain may be obtained. Alternatively, a selenocysteine residue may be introduced at the C-terminal of the polypeptide chain, in a similar fashion as for the introduction of a cysteine residue, to facilitate site-specific conjugation (Cheng et al, Nat Prot 1:2, 2006).

In one embodiment, the albumin binding domain comprises no more than two cysteine residues. In another embodiment, the albumin binding domain comprises no more than one cysteine residue.

### Generally Applicable Aspects of Moiety (II)

In some embodiments, the albumin binding domain within moiety (II) in the compound for use according to the invention binds to albumin such that the K_{D} value of the interaction is at most 1×10⁻⁸ M, i.e. 10 nM. In some embodiments, the K_{D} value of the interaction is at most 1×10⁻⁹ M, at most 1×10⁻¹⁰ M, at most 1×10⁻¹¹ M, or at most 1×10⁻¹² M.

In one embodiment, the albumin binding domain within moiety (II) binds to human serum albumin. In one embodiment, the albumin binding domain instead or additionally binds to albumin from other species than the human species, such as albumin from mouse, rat, dog and cynomolgus macaques.

As explained extensively above, the albumin binding moiety (II) may comprise an amino acid sequence selected from SEQ ID NO:258-718 or a subset thereof, or comprise, as an albumin binding motif in a larger albumin binding domain, a sequence selected from SEQ ID NO:1-257. As the skilled person will realize, the function of any polypeptide, such as the albumin binding capacity of these polypeptide domains, is dependent on the tertiary structure of the polypeptide. It is however possible to make changes to the sequence of amino acids in an α-helical polypeptide without affecting the structure thereof (Taverna and Goldstein, J Mol Biol 315(3):479-84, 2002; He et al, Proc Natl Acad Sci USA 105(38):14412-17, 2008). Thus, modified variants of the naturally occurring albumin proteins or of the derivatives thereof as disclosed in detail above are also envisaged as candidates for the albumin binding domain comprised in moiety (II). For example, it is possible that an amino acid residue belonging to a certain functional grouping of amino acid residues (e.g. hydrophobic, hydrophilic, polar etc) could be exchanged for another amino acid residue from the same functional group.

Thus, moiety (II) may comprise variants of the disclosed albumin binding proteins which exhibit small differences only in comparison with SEQ ID NO:1-718. One such definition is an albumin binding domain having an amino acid sequence with at least 85% identity to a sequence selected from SEQ ID NO:258-718. In some embodiments, the albumin binding domain may have a sequence which has at least 86%, at least 87%, at least 88%, at least 89%, at least 90%, at least 91%, at least 92%, at least 93%, at least 94%, at least 95%, at least 96%, at least 97%, at least 98% or at least 99% identity to the sequence selected from SEQ ID NO:258-718.

The term “% identical” or “% identity”, as used in the specification and claims, is calculated as follows. The query sequence is aligned to the target sequence using the CLUSTAL W algorithm (Thompson, J. D., Higgins, D. G. and Gibson, T. J., Nucleic Acids Research, 22: 4673-4680 (1994)). A comparison is made over the window corresponding to the shortest of the aligned sequences. The shortest of the aligned sequences may in some instances be the target sequence, such as the albumin binding domain disclosed herein. In other instances, the query sequence may constitute the shortest of the aligned sequences. The query sequence may for example consist of at least 10 amino acid residues, such as at least 20 amino acid residues, such as at least 30 amino acid residues, such as at least 40 amino acid residues, for example 45 amino acid residues. The amino acid residues at each position are compared, and the percentage of positions in the query sequence that have identical correspondences in the target sequence is reported as % identity.

The terms “albumin binding” and “binding affinity for albumin” as used in this specification refer to a property of a polypeptide which may be tested for example by the use of surface plasmon resonance technology, such as in a Biacore instrument. For example as described in the examples below, albumin binding affinity may be tested in an experiment in which albumin, or a fragment thereof, is immobilized on a sensor chip of the instrument, and the sample containing the polypeptide to be tested is passed over the chip. Alternatively, the polypeptide to be tested is immobilized on a sensor chip of the instrument, and a sample containing albumin, or a fragment thereof, is passed over the chip. Albumin may, in this regard, be a serum albumin from a mammal, such as human serum albumin. The skilled person may then interpret the results obtained by such experiments to establish at least a qualitative measure of the binding affinity of the polypeptide for albumin. If a quantitative measure is desired, for example to determine a K_{D} value for the interaction, surface plasmon resonance methods may also be used. Binding values may for example be defined in a Biacore2000 instrument (GE Healthcare). Albumin is suitably immobilized on a sensor chip of the measurement, and samples of the polypeptide whose affinity is to be determined are prepared by serial dilution and injected. K_{D} values may then be calculated from the results using for example the 1:1 Langmuir binding model of the BIAevaluation 4.1 software provided by the instrument manufacturer (GE Healthcare).

### II Pharmaceutical Composition

In a second aspect, the invention provides a pharmaceutical composition for oral administration, comprising:

a) a compound, which comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment; and

b) at least one pharmaceutically acceptable excipient.

Thus, this second aspect of the invention provides a pharmaceutical composition which comprises as component a) a compound as defined in connection with the first aspect of the invention. When present in a pharmaceutical composition for oral administration, this compound may exhibit any one or more of the properties, features, characteristics and/or embodiments described above in connection with the first aspect of the invention, in any combination. For the sake of brevity, this information will not be repeated verbatim in connection with this second aspect, but is incorporated by reference to the above disclosure.

The pharmaceutical composition also comprises b) at least one pharmaceutically acceptable excipient. “Excipients” are inert substances used as diluents or vehicles in a drug formulation. It is mixed with the therapeutically active compound or compounds to facilitate administration or manufacture, improve product delivery, promote the consistent release and bioavailability of the drug, enhance stability, assist in product identification, or enhance other product characteristics. Excipients may be classified into binders, diluents/fillers, lubricants, glidants, disintegrants, polishing agents, colorings, suspending agents, film formers and coatings, plasticizers, dispersing agents, preservatives, flavorings, sweeteners etc.

In some embodiments of the inventive pharmaceutical composition, it further comprises at least one component for increasing oral bioavailability of the moiety (I) which confers a desired therapeutic activity. In those embodiments, the component in question may be selected from the group consisting of protease inhibitors, absorbance enhancers, mucoadhesive polymers, formulation vehicles and any combination thereof. Uses of such components and the scientific rationale behind them are described in the following sections, concerning general strategies to improve the oral bioavailability of therapeutics.

The resistance of the pharmaceutical composition to the acid and enzymatic environment of the gastrointestinal tract may be increased by adding one or more inhibitors (cocktails or individually targeting) of the relevant peptide- and protein-targeting enzymes active in the stomach (e.g. pepsin) and the intestine (e.g. trypsin, chymotrypsin and carboxypeptidase). Such inhibitors may be selected from trypsin and α-chymotrypsin inhibitors such as pancreatin inhibitor, soybean trypsin inhibitor, FK-448, camostat mesylate, aprotinin, chicken and duck ovomucoids, carboxymethylcellulose and Bowman-Birk inhibitor; or mucoadhesive polymer protease-inhibitor conjugates (Park et al, Reactive and Functional Polymers, 71:280-287, 2011).

To increase the absorption of polypeptides though the intestinal wall and hence improve the therapeutic efficacy, absorbance enhancers rendering the epithelial barrier more permeable may be included in the pharmaceutical composition. The absorbance enhancers may for instance disrupt the lipid bilayer of the cell membrane improving the transcellular transport, or act as chelating agents rupturing tight junctions facilitating paracellular transport. Non-limiting examples of absorbance enhancers for use in this aspect of the invention are detergents, surfactants, bile salts, calcium chelating agents, fatty acids, medium chain glycerides, salicylates, alkanoyl cholines, N-acetylated α-amino acids, N-acetylated non-α-amino acids, chitosans, phospholipids, sodium caprate, acyl carnitine and Zonula Occludens toxin (Park et al, 2011, supra; Salama et al, Adv Drug Deliv Rev. 58:15-28, 2006).

As an additional or alternative component in the pharmaceutical composition, mucoadhesive polymers have the potential to protect from proteolytic degradation, but are primarily applied to provide site-specific delivery to the mucus membrane, extend the residence time at the site of drug absorption and to improve membrane permeation, all promoting increased absorbance through the intestinal wall. Non-limiting examples for use in the inventive pharmaceutical composition are poly(methacrylic acid-g-ethylene glycol)[P(MAA-g-EG)] hydrogel microparticles, lecithin conjugated alginate microparticles, thiolated polymers (thiomers), gastrointestinal mucoadhesive patch systems (GI-MAPS) and mucoadhesive polymer protease-inhibitor conjugates (Park et al, 2011, supra).

Formulation vehicles, such as emulsions, liposomes, microspheres, nanospheres, nanocapsules or complete encapsulation, may contribute to the protection from proteolytic degradation and provide a controlled release rate, as well as promoting enhanced delivery across the intestinal wall. Such formulation vehicles constitute yet an alternative or complementary component for use in the inventive pharmaceutical composition. In particular, nanoparticles having modified surface properties or being coupled to a targeting molecule may be used. Surface modification of nanoparticles can for example be achieved either by coating with hydrophilic stabilizing, bioadhesive polymers or surfactants, or by incorporating hydrophilic copolymers in the nanoparticle formulation. Examples of such hydrophilic polymers include PEG and chitosan (des Rieux et al, J Control Release. 116:1-27, 2006). Targeting nanoparticles are designed to specifically adhere to receptors expressed on enterocytes or M-cells of the epithelial layer of the intestinal wall by for instance coupling ligands such as lectins or RGD (arginine-glycine-aspartate) derivatives to the nanoparticle (des Rieux et al, 2006, supra). M-cells also provide a route for delivery into the lymphatic system (Rubas and Grass, Advanced Drug Delivery Reviews, 7:15-69, 1991).

The pharmaceutical composition of the invention may for example be orally administered in solid form, such as in pills, tablets, capsules, powders or granules; in semi-solid form, such as in pastes; or in liquid form, such as in elixirs, solutions or suspensions. Solid forms are currently preferred, and may contain excipients such as chitosan, alginates, microcrystalline cellulose, lactose, saccharose, starch, gelatin, milk sugar, polyethylene glycols, polyvinylpyrrolidone (PVP), magnesium stearate, calcium stearate and sodium starch glycolate. Preparations in liquid forms may contain excipients such as sweetening or flavoring agents, emulsifying or suspending agents or diluents such as water, ethanol, propylene glycol and glycerin.

The formulation may be intended for immediate-, delayed- or controlled-release applications. Tablets or capsules intended for immediate release should rapidly disintegrate and release the entire active substance in the upper part of the GI tract, i.e. the stomach. On the contrary, tablets or capsules intended for delayed or controlled release can be designed for time-dependent release (depot) or site-specific release (e.g. intestine). Time-dependent release may for instance be based on dissolution or diffusion controlled release dependent on the matrix or membrane composition. Site-specific release may for instance be based on pH- or enzyme sensitivity. Particularly preferred for formulation of the pharmaceutical composition according to the invention are enteric-coated capsules, intended for release in the small intestine or colon. Such enteric-coated capsules should be stable at the highly acidic pH of the stomach, but be rapidly dissolved at the less acidic pH of the intestinal tract. Examples of pH sensitive enteric film forming agents include cellulose polymers such as hydroxypropyl methyl cellulosephthalate (HPMCP), cellulose acetate phthalate (CAP), cellulose acetate trimellitate (CAT), hydroxypropyl methyl acetatesuccinate (HPMCAS), polyvinyl acetate phthalate (PVAP), and other polymers such as Eudragit® derivatives, shellac (SH), chitosan and chitin.

### III Method of Treatment

In a third aspect, the invention provides a method of treatment of a mammalian subject in need of such treatment, comprising oral administration of a compound, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment.

Thus, this third aspect of the invention provides a method of treatment which comprises orally administering a compound as defined in connection with the first aspect of the invention. Optionally, this compound may be administered present in a pharmaceutical composition as defined in connection with the second aspect of the invention. The compound and pharmaceutical composition, respectively, may each individually exhibit any one or more of the properties, features, characteristics and/or embodiments described above in connection with the first and second aspects of the invention, in any combination. For the sake of brevity, this information will not be repeated verbatim in connection with this third aspect, but is incorporated by reference to the above disclosure.

In one embodiment, the method of treatment according to the invention is carried out according to a specified dosage regime. The optimal dosage regime will depend on the potency of the moiety conferring the therapeutic effect, on the bioavailability of the compound as defined herein and on the nature of the disease to be treated. However, the compound as defined herein, which comprises an albumin binding moiety (II) that is thought to extend the half-life of the compound, would not require a single high dose to reach the level of a therapeutic effect, but, due to the sustained residence time in the circulation, allows for administration of lower repeated doses leading to a build-up of the concentration of the compound, eventually reaching a sustainable desired therapeutic effect. In other words, following oral administration, a lower bioavailability than for a short-lived therapeutic would be acceptable. Such repeated dosing may be given at least twice monthly, once weekly, twice weekly, three times weekly, once daily, twice daily, such as at least three times daily.

For certain diseases it may be desirable to administer a bolus dose, followed by repeated lower doses. The bolus dose may be taken as multiples of an orally formulated drug, at least once daily, twice daily, three times daily, four times daily or at least five times daily. Alternatively, the high bolus dose may be administered via another route, such as by an intravenous or subcutaneous injection. Subsequent dosing, serving the purpose of providing a sustained therapeutic effect, may be given at least twice monthly, once weekly, twice weekly, three times weekly, once daily, twice daily, three times daily, such as at least four times daily.

### DEFINITIONS AND USE OF TERMS

In the present text, “bioavailability” refers to the fraction of an administered dose of an active drug substance that reaches the systemic circulation. By definition, the bioavailability of an intravenously administered drug is 100%. However, when the drug is administered via other routes, for instance by the oral route, the bioavailability decreases due to metabolism and incomplete absorbance. Absolute bioavailability compares the bioavailability of the active drug in systemic circulation following non-intravenous administration with the bioavailability of the same drug following intravenous administration. It is calculated as the fraction of the drug absorbed through non-intravenous administration compared with the corresponding intravenous administration of the same drug. The comparison must be normalized (e.g. account for different doses or varying weights of the subjects). In order to determine absolute bioavailability of a drug, a pharmacokinetic study must be performed to obtain a plasma drug concentration versus time plot for the drug after both intravenous and non-intravenous administration. The absolute bioavailability is the dose-corrected area under curve (AUC) non-intravenous divided by AUC intravenous.

The invention will now be further illustrated by the following non-limiting Examples.

### BRIEF DESCRIPTION OF THE FIGURES

FIG. 1 is a table providing an informal listing of the various amino acid sequences discussed in the present text.

FIG. 2 shows the result of SDS-PAGE analysis of purified polypeptide variants produced as described in Example 1. Lane 1-2: 25 and 50 μg, respectively, of PEP04419; lane 3-4: 25 and 50 μg, respectively, of PEP10986; and lane 5-6: 25 and 50 μg, respectively of PEP03973. Lane M: Novex®Sharp pre-stained protein standard (molecular weights: 3.5, 10, 15, 20, 30, 40, 50, 60, 80, 110, 160 and 260 kDa).

FIG. 3 shows the pharmacokinetic profiles after oral administration of PEP03973 (open squares), PEP10986 (open triangles) and PEP04419 (open circles), respectively, in mice as described in Example 2. FIG. 3A shows the concentrations in serum measured over time (mean of three animals per time-point). FIG. 3B represents the same data as in A but adjusted for variation in administered dose of the three polypeptides (i.e. at each time-point: [measured serum concentration]/[administered dose]).

FIG. 4 shows the pharmacokinetic profile of PEP10896 in serum samples obtained from rat after oral gavage (open squares) or intraduodenal administration (open circles) as described in Example 3. The concentration of PEP10896 was determined by ELISA and mean nM+/−SD values are presented.

FIG. 5 shows the pharmacokinetic profile after repeated intraduodenal administration of PEP10896 as described in Example 4. The polypeptide was given at time points zero, 2 and 24 hours, marked in the graph by arrows. The concentration of PEP10896 was determined by ELISA and the mean nM+/−SD values are shown (open circles). The pharmacokinetic profile after single intraduodenal administration as determined in Example 3 is shown for comparison (open squares).

### EXAMPLES

### Example 1

### Cloning, Production and Characterization of Polypeptides

### Materials and Methods

For illustration of the invention, three polypeptides denoted PEP03973, PEP10986 and PEP04419, respectively, were prepared for oral administration in mice. PEP03973 and PEP10986 each comprise a different albumin binding moiety, which has been N-terminally fused to a variant of protein Z (derivative of domain B of staphylococcal protein A; Nilsson B et al, 1987, supra), whereas PEP04419 is a variant of protein Z which has been C-terminally conjugated to MMA-DOTA (maleimide-monoamide-1,4,7,10-tetraazacyclododecane-1,4,7,10-tetraacetic acid) as previously described in Feldwisch et al (J. Mol. Biol. 398: 232-247, 2010; there denoted ABY-025). Thus, PEP04419 does not comprise any albumin binding moiety and is tested for comparison. PEP03973 comprises the wild-type albumin binding domain (ABDwt, SEQ ID NO:515; i.e. the GA3 domain of protein G from Streptococcus strain G148; Kraulis et al, 1996, supra; Johansson et al, 2002, supra), whereas PEP10986 comprises a derivative of this GA3 domain (SEQ ID NO:528).

Cloning and Cultivation of polypeptide Variants:

DNA encoding the polypeptides PEP03973 and PEP10986, respectively, were cloned into expression vectors containing a T7 promoter, a multiple cloning site and a kanamycin resistance gene, using standard molecular biology techniques. The expression vector encodes the amino acids GSSLQ N-terminally of the Z variant sequences, and the Z variant and the albumin binding domain sequences are separated by the amino acids VD and VDSS in PEP03973 and PEP10986, respectively.

E. coli BL21(DE3) cultures transformed with plasmids for expression of PEP03973 and PEP10986, respectively, were inoculated into 800 ml TSB+YE medium supplemented with 50 μg/ml kanamycin and 0.3 ml/I anti-foam agent (Breox FMT 30) and grown at 37° C. to an OD₆₀₀ of approximately 2. Protein expression was then induced by addition of 1 M IPTG to a final concentration of 0.2 mM. The cultivations were performed using the multifermentor system Hedvig (Belach Bioteknik, Stockholm, Sweden). The cultures were harvested 5 h after induction by centrifugation at 15900×g for 20 min. Supernatants were discarded and the cell pellets collected and stored at −20° C. The protein expression level was determined using SDS-PAGE and ocular inspection of stained gels.

Purification of Polypeptide Variants:

Pelleted bacterial cells harboring soluble PEP03973 and PEP10986, respectively, were suspended in TST-buffer (25 mM Tris-HCl, 1 mM EDTA, 200 mM NaCl, 0.05% Tween 20, pH 8) supplemented with 20 U/ml Benzonase® and disrupted by ultra sonication. The lysates were clarified by centrifugation and loaded on 100 ml affinity agarose packed in an XK50 column (GE Healthcare), pre-equilibrated with TST-buffer. After column wash with 6 column volumes (CV) TST-buffer, followed by washing with 6 CV 5 mM NH₄Ac pH 5.5, bound proteins were eluted with 3 CV 0.1 M HAc. The flow rate was 15 ml/min and the 280 nm signal was monitored. Fractions containing PEP03973 and PEP10986, respectively, were identified by SDS-PAGE analysis. Relevant fractions were pooled and acetonitrile (ACN) was added to a final concentration of 10% and loaded on a FineLine column packed with 125 ml Source 15 RPC (GE Healthcare), pre-equilibrated with RPC Eluent A (0.1% TFA, 10% ACN, 90% water). After column wash with 5 CV RPC Eluent A, bound proteins were eluted with a linear gradient 0-60% RPC Eluent B (0.1% TFA, 80% ACN, 20% water) during 10 CV. The flow rate was 30 ml/min and the signal at 280 nm was monitored. Fractions containing pure PEP03973 and PEP10986, respectively, were identified by SDS-PAGE analysis and separately pooled.

Purified PEP03973 and PEP10986 were transferred to 50 mM NaAc pH 4.5 and 50 mM sodium phosphate pH 7.0, respectively, by buffer exchange using 500 ml Sephadex G25m (GE Healthcare) packed in a XK50 column (GE Healthcare). Finally, concentration was performed using 15 ml Amicon Ultra centrifugal filter units with 3 kDa MWCO (Millipore).

PEP04419 was produced essentially as described in Feldwisch et al (J. Mol. Biol. 2010, supra). Purified PEP04419 was transferred to 25 mM NH₄Ac, 6.25 mM HCl, 112.5 mM NaCl, pH 4.9 by buffer exchange and concentrated as described for PEP03973 and PEP10986 above.

### Analysis of Purified Polypeptide Variants:

Determination of protein concentration was performed by measuring the absorbance at 280 nm using a NanoDrop® ND-1000 spectrophotometer.

For the SDS-PAGE analysis, concentrated PEP03973, PEP10986 and PEP04419 were diluted to 5 mg/ml and mixed with 4xLDS Sample Buffer, incubated at 70° C. for 15 min and loaded onto a 10 well NuPAGE® 4-12 Bis-Tris Gel. The gel was run with MES SDS Running Buffer in a Novex Mini-Cell employing the Novex®Sharp pre-stained protein standard as molecular weight marker and Coomassie blue for staining.

To verify the identity of the purified PEP03973, PEP10986 and

PEP04419, LC/MS-analyses were performed using an Agilent 1100 LC/MSD system, equipped with API-ESI and single quadruple mass analyzer. 25 μg was loaded on a Zorbax 300SB-C8 Narrow-Bore column (2.1×150 mm, 3.5 μm) at a flow-rate of 0.5 ml/min. Proteins were eluted using a linear gradient of 10 to 70% of Eluent B over 15 min at 0.5 ml/min. The separation was performed at 30° C. The ion signal and the absorbance at 280 and 220 nm were monitored. The molecular weights of the purified proteins were determined by analysis of the ion signal.

### Results

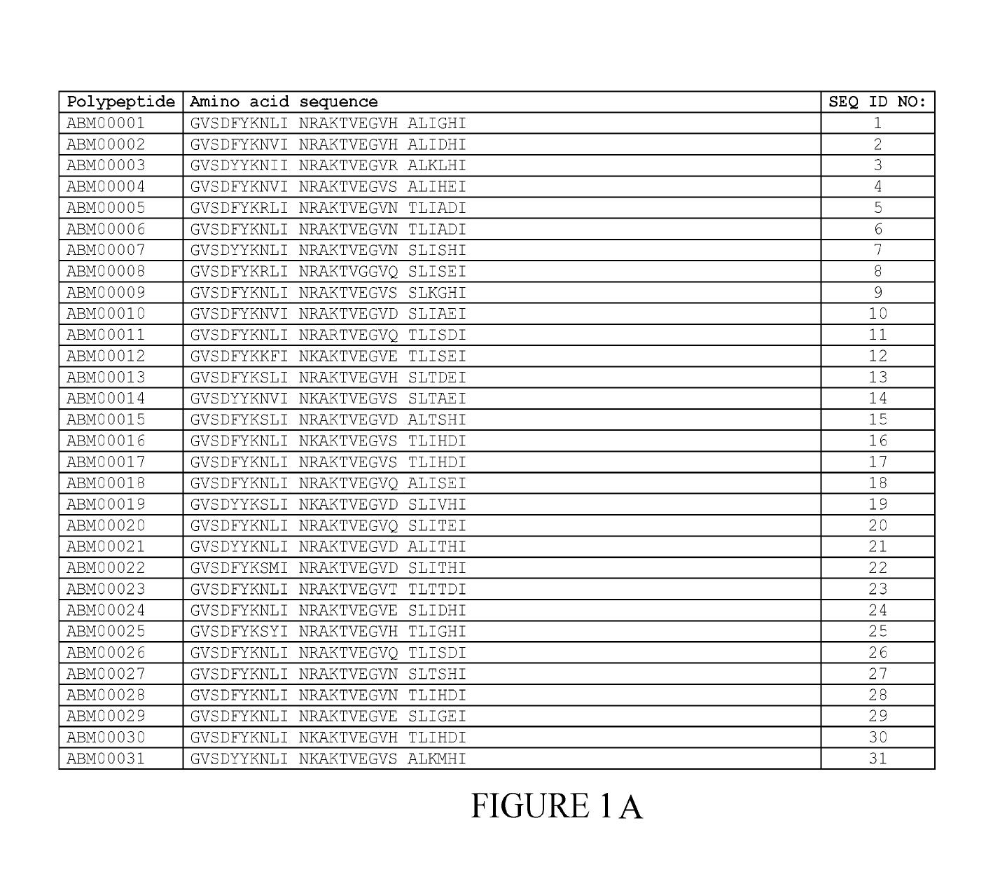

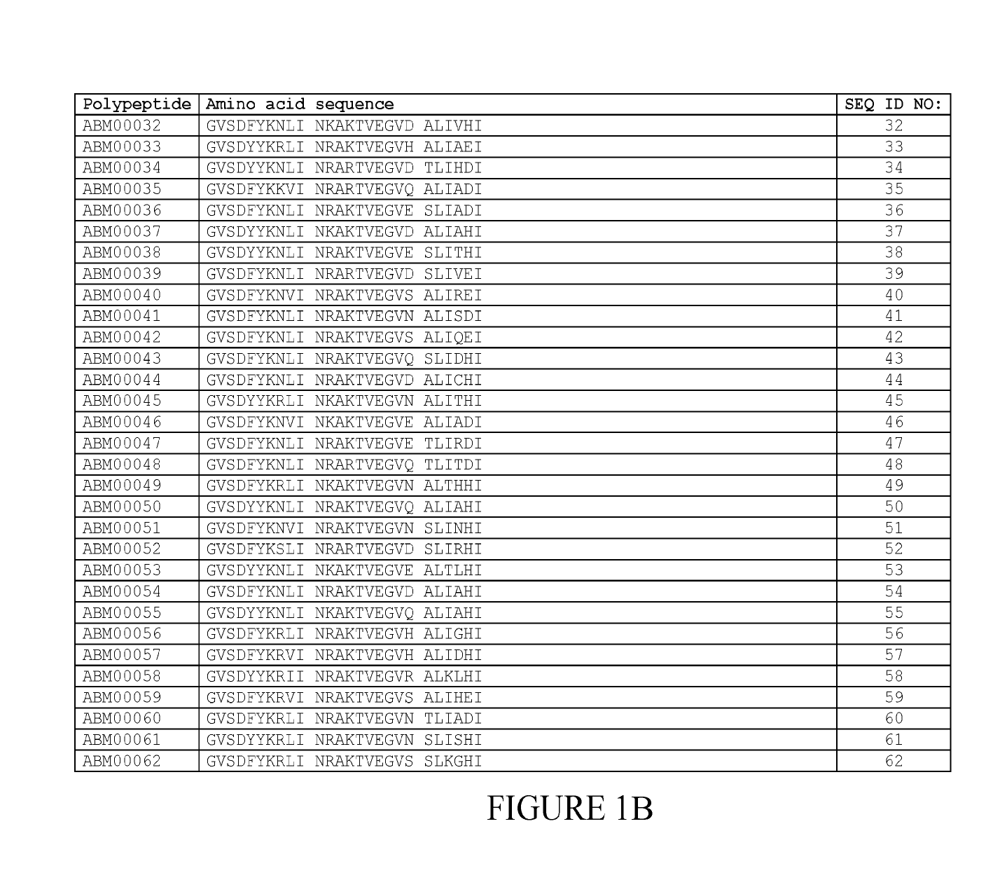

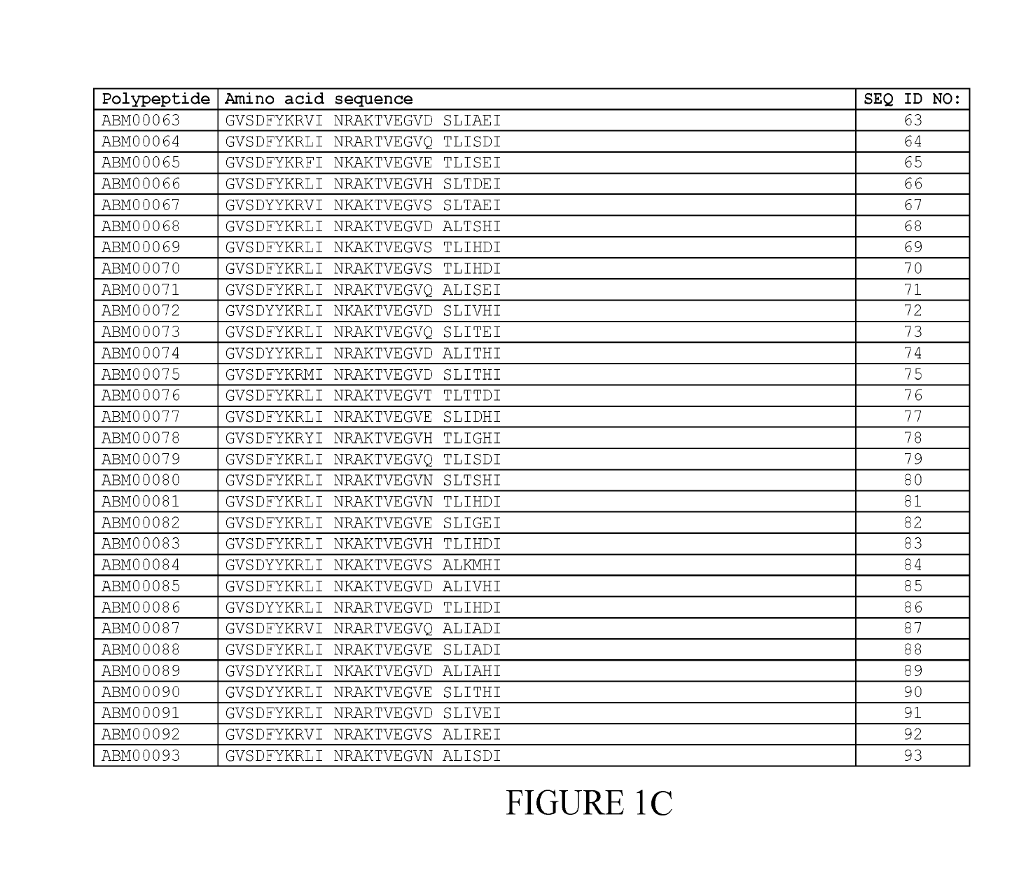

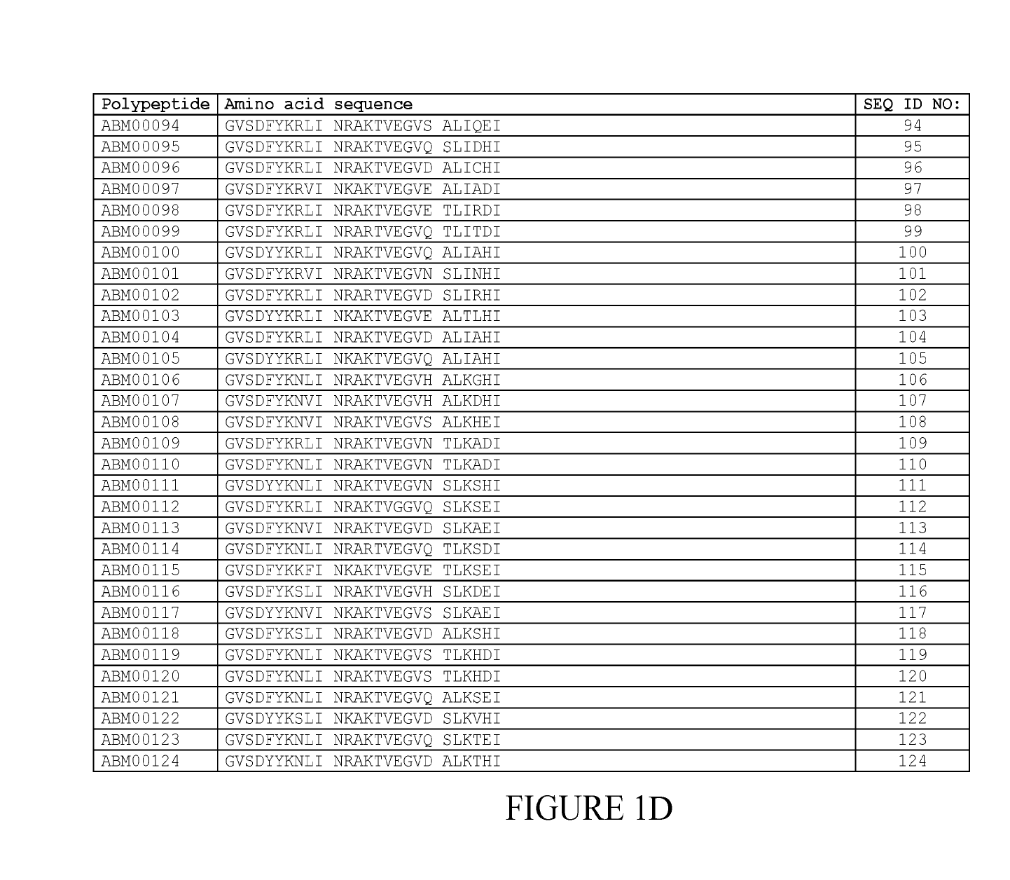

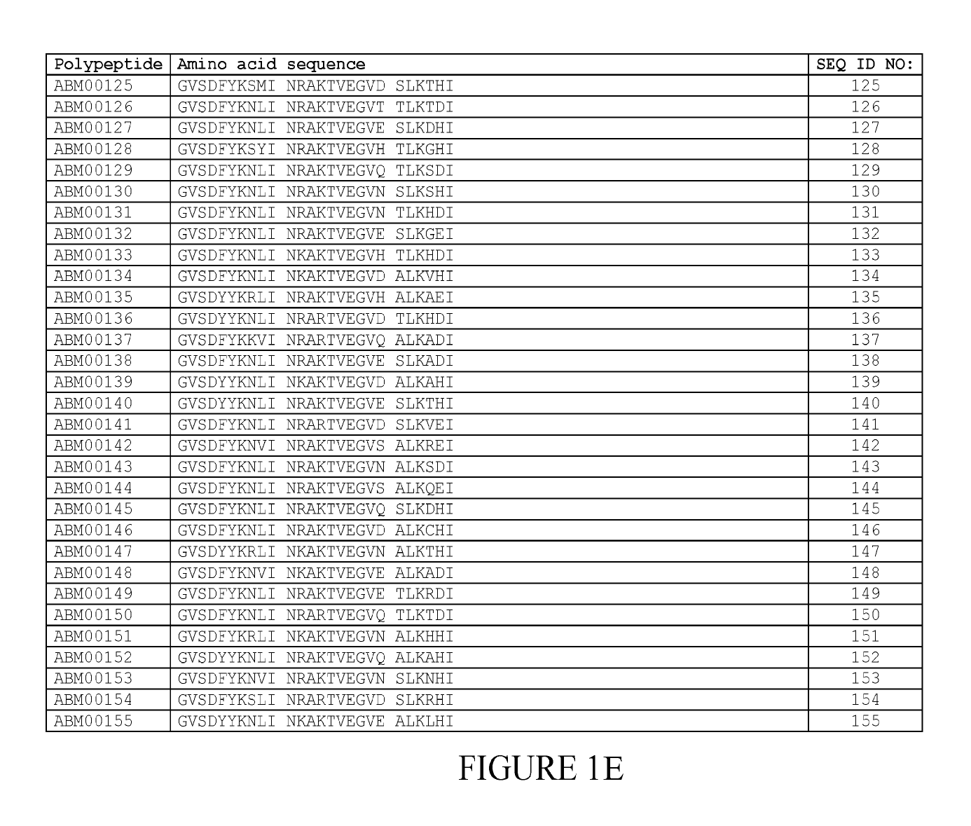

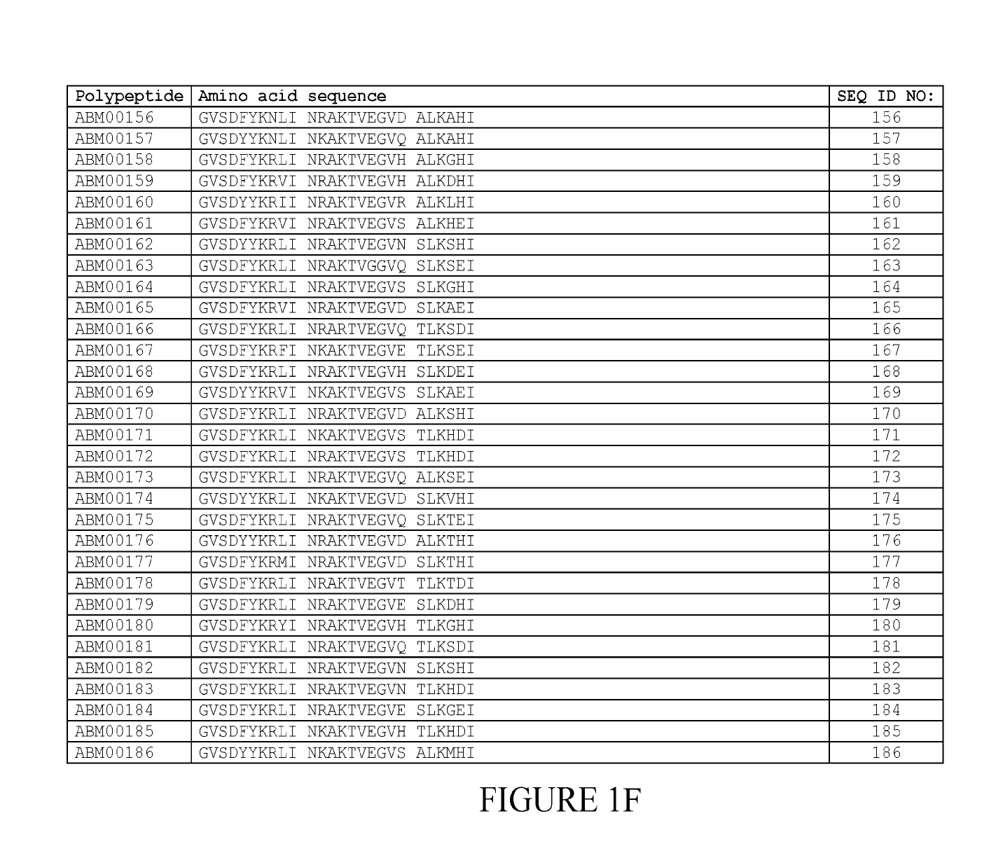

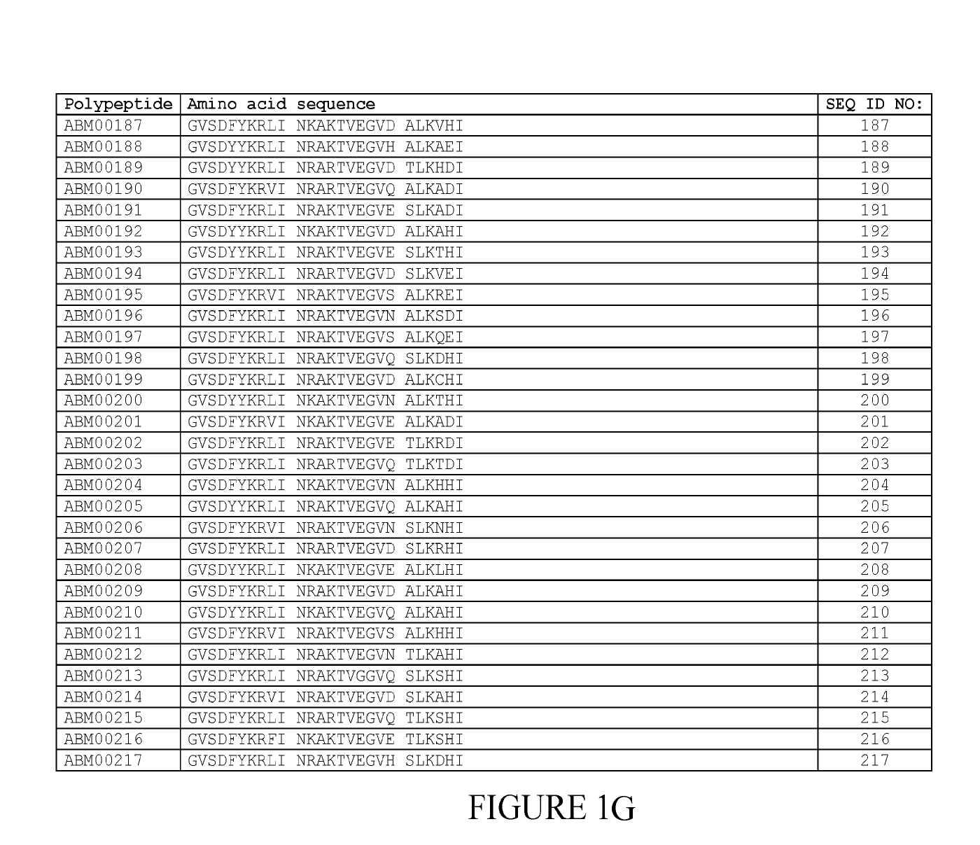

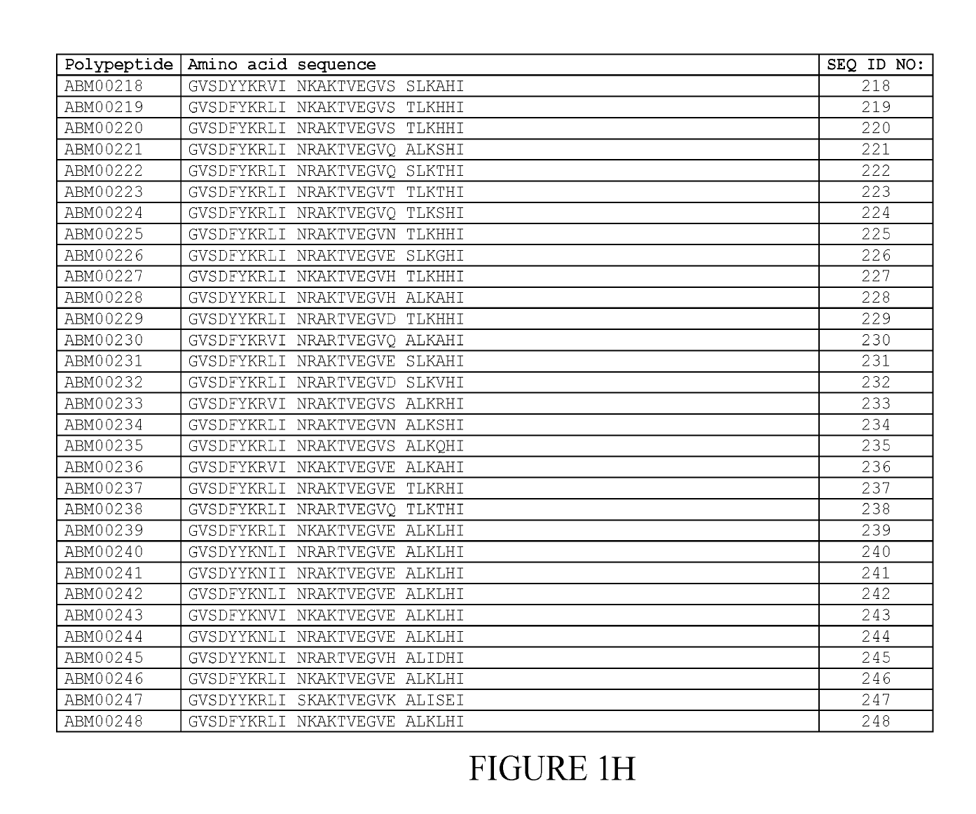

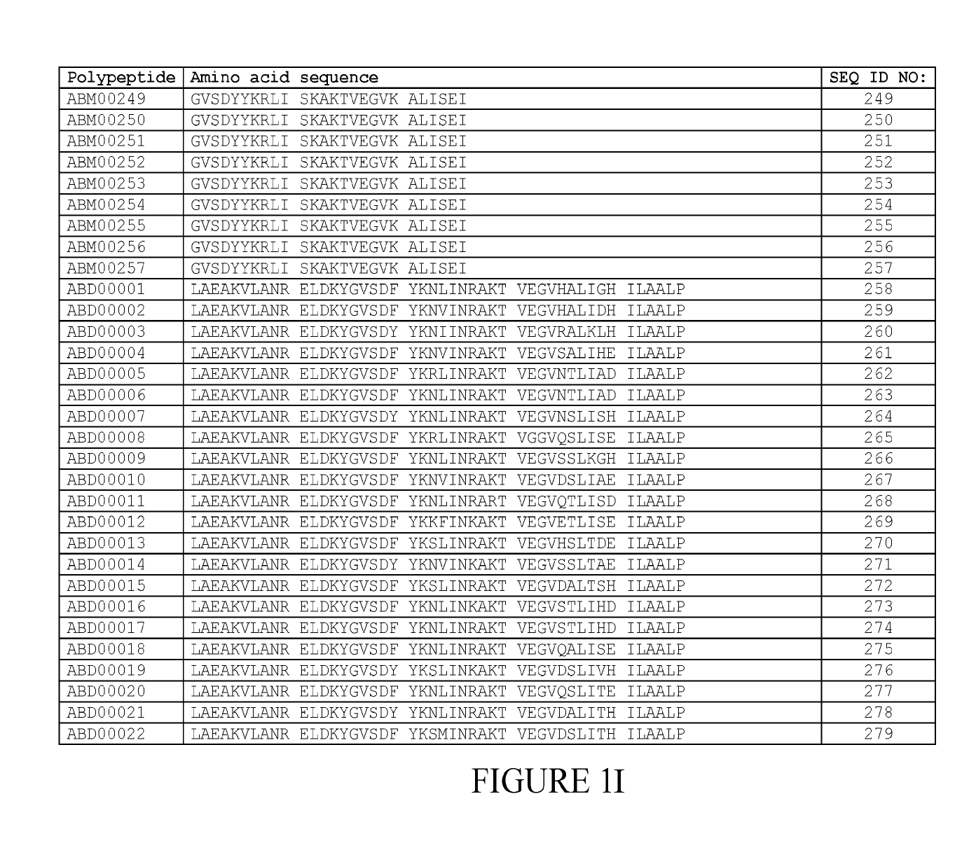

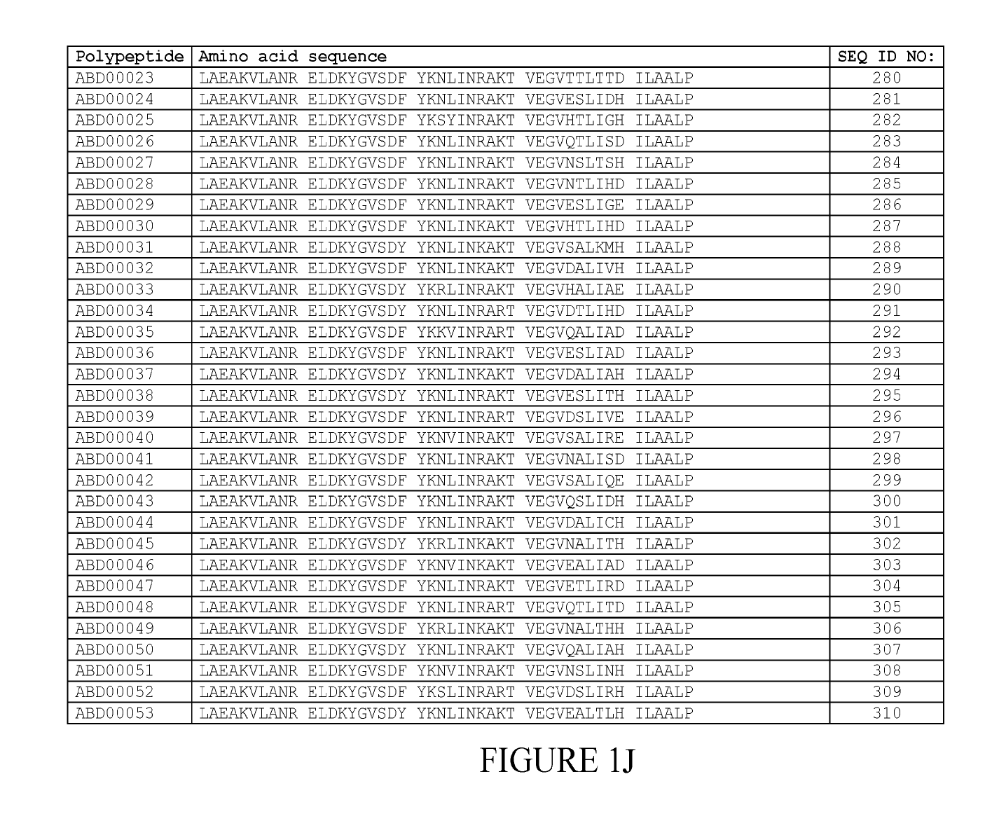

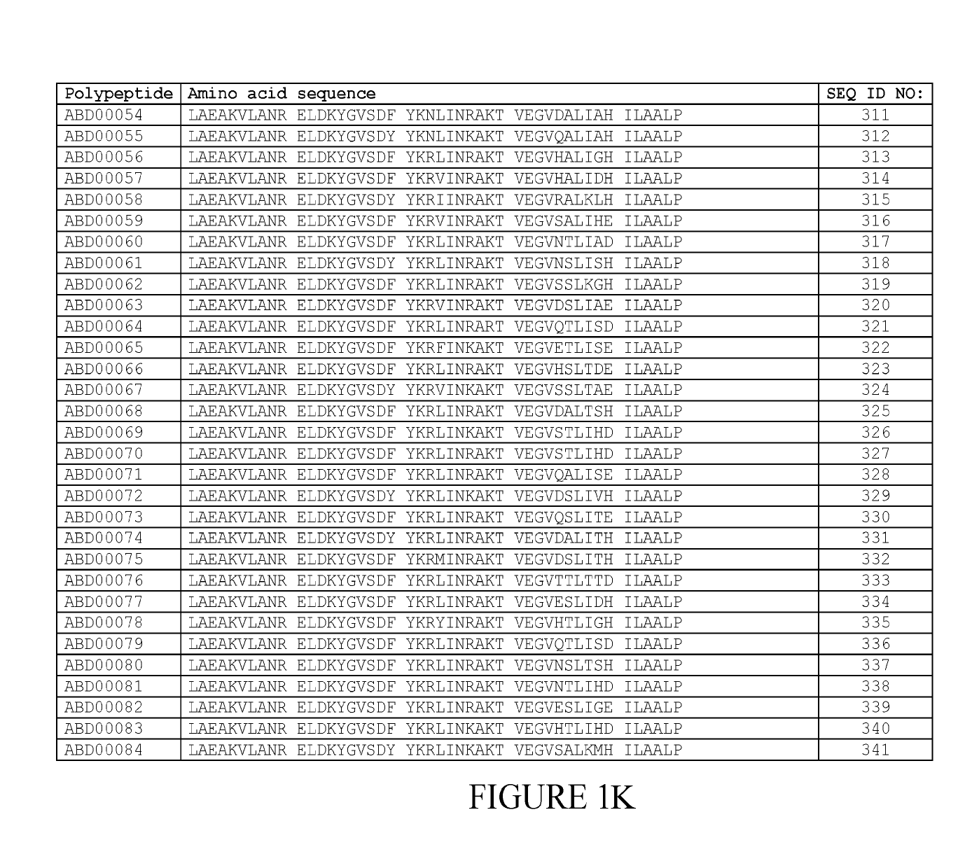

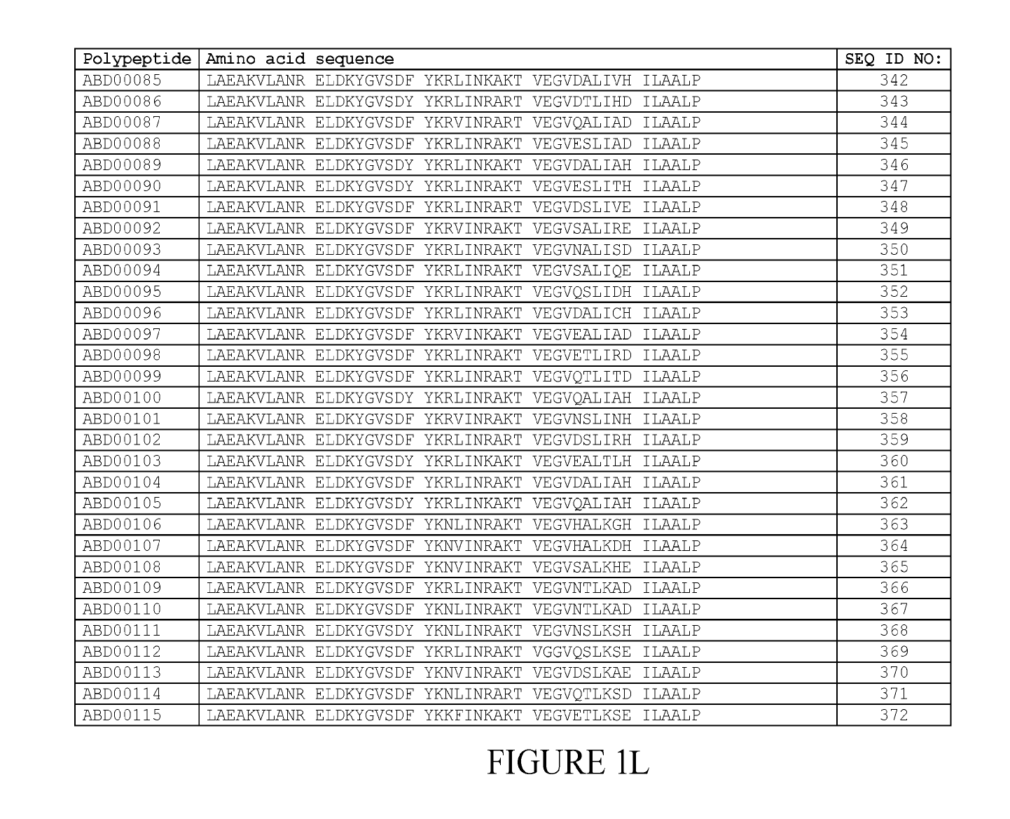

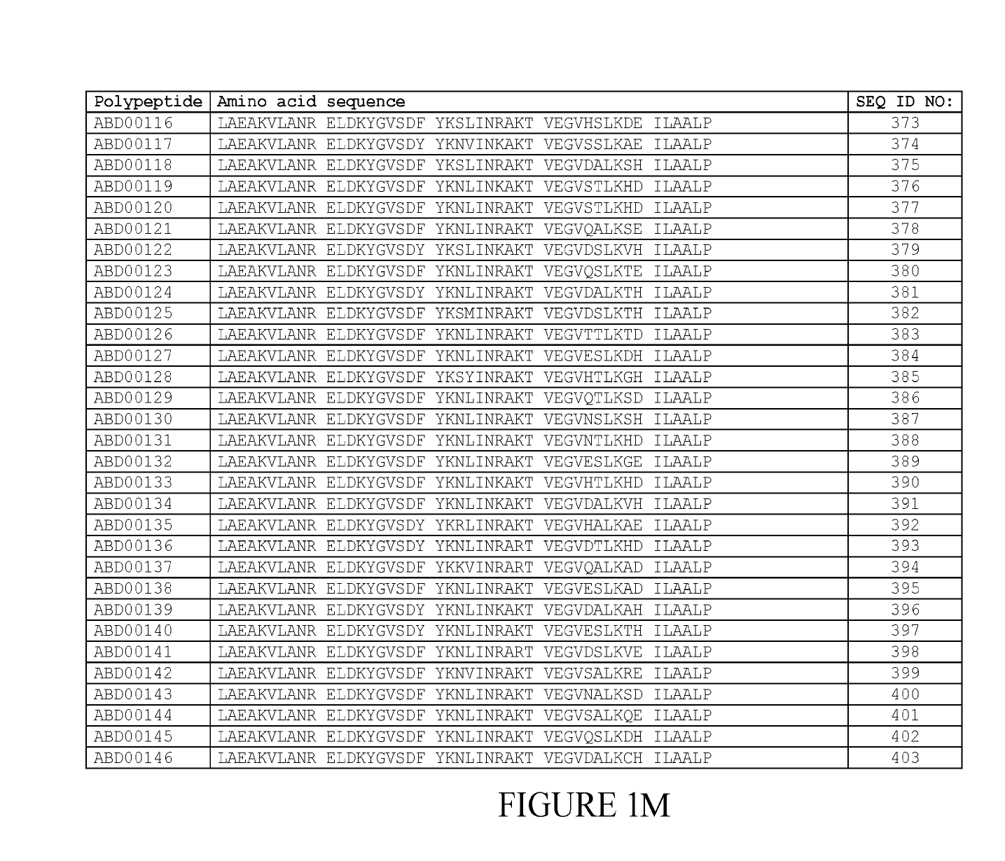

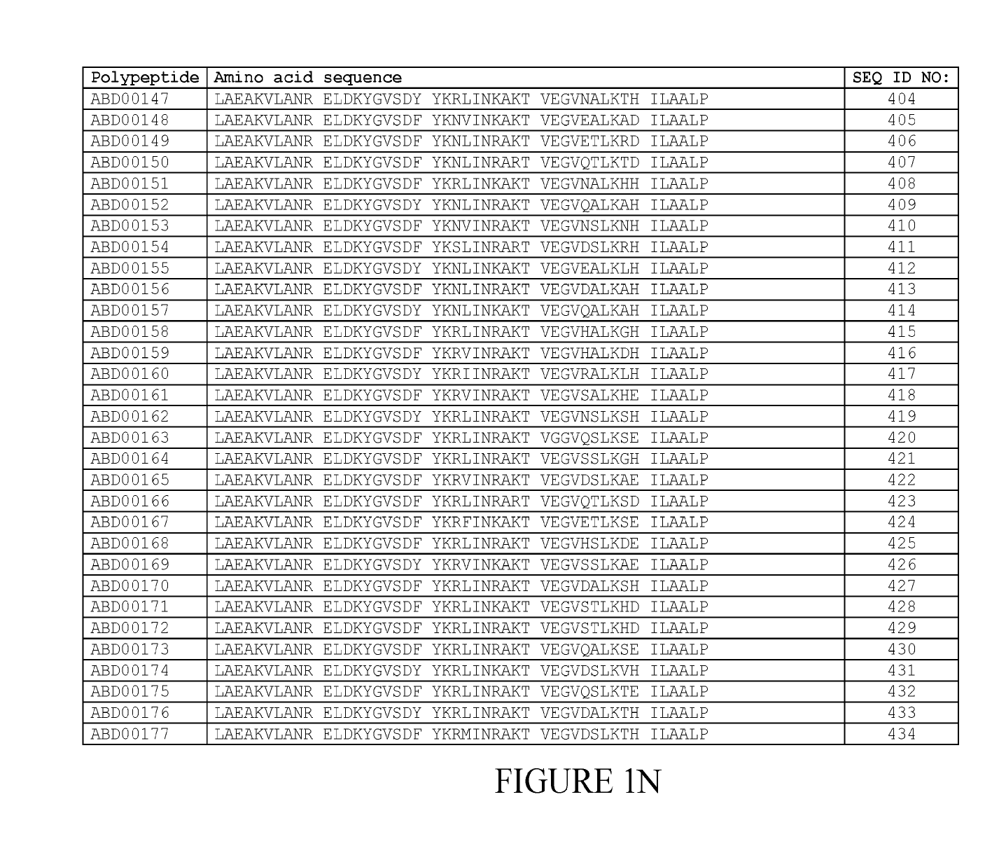

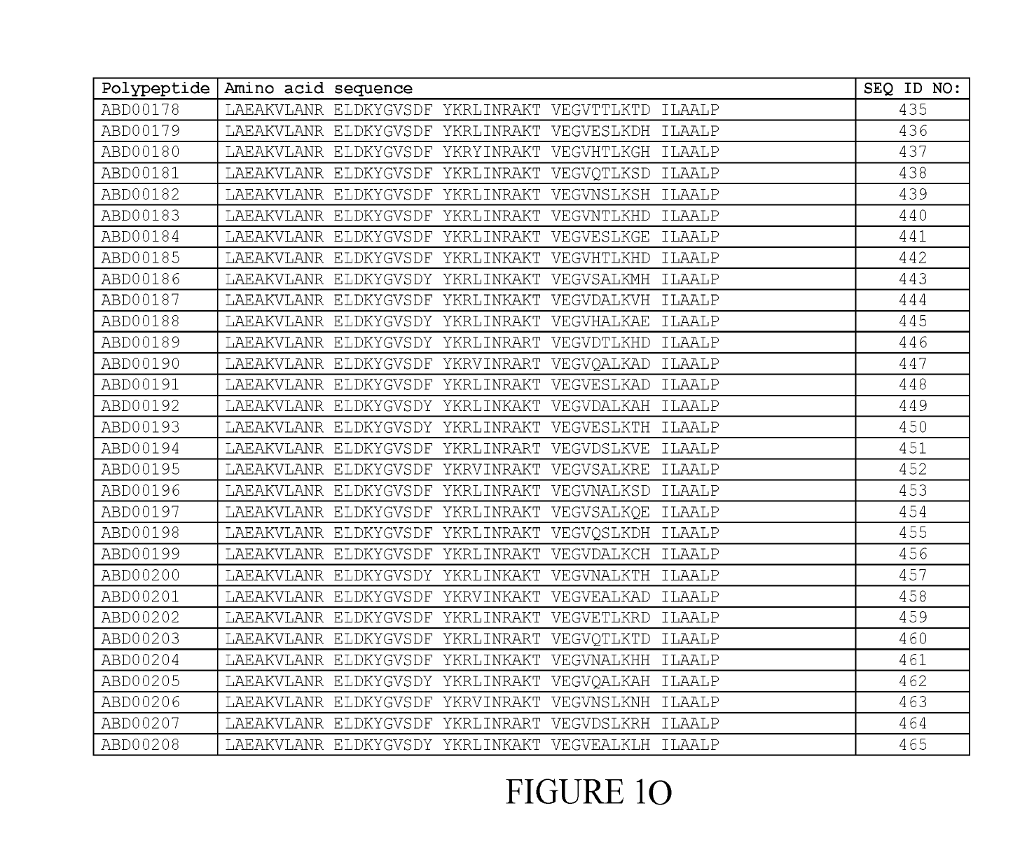

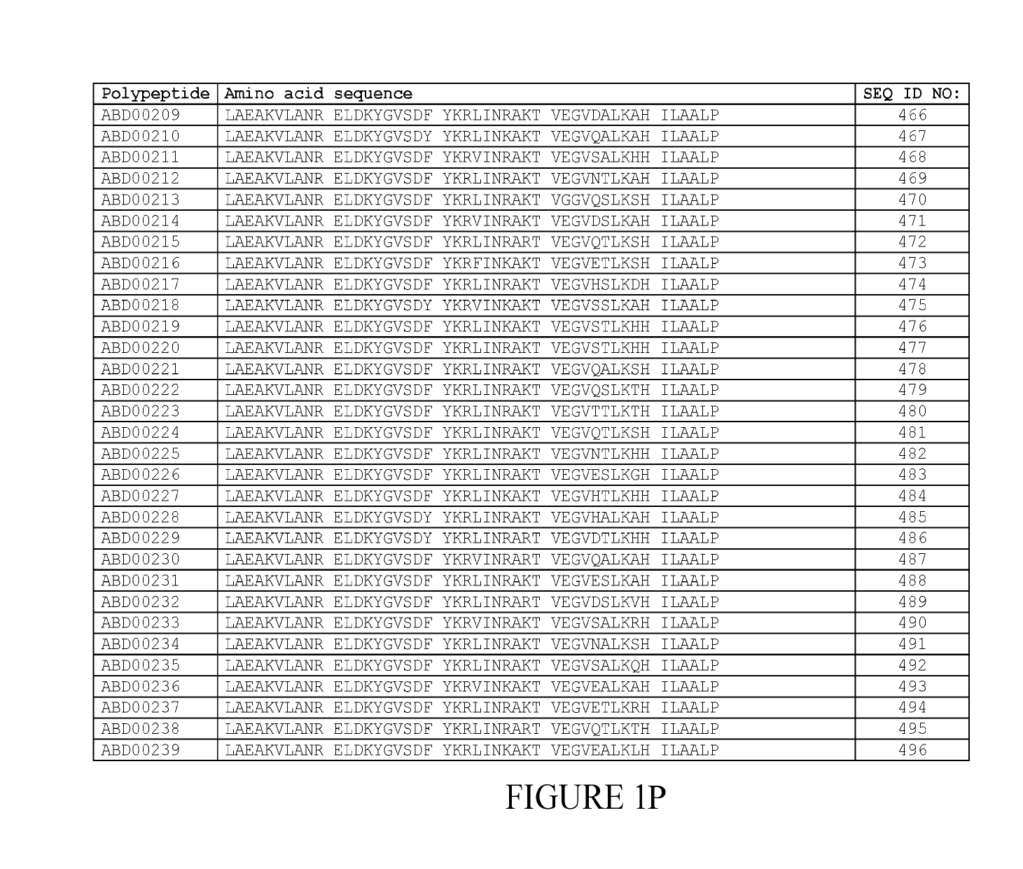

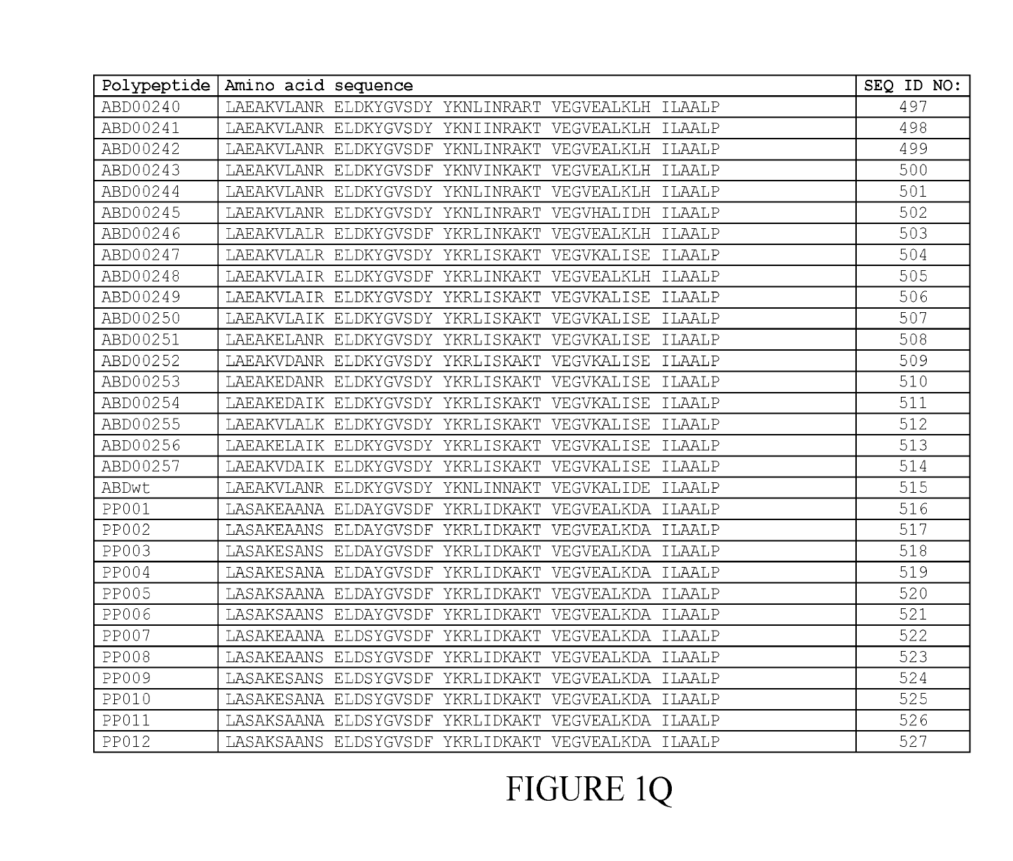

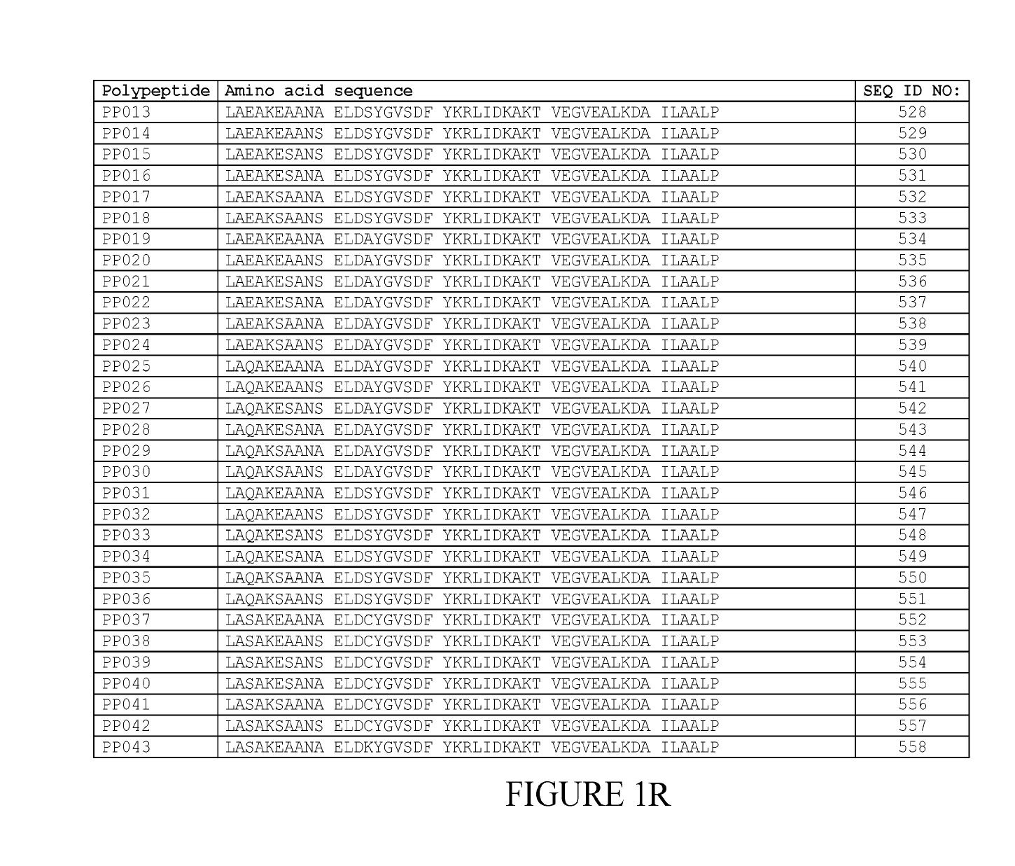

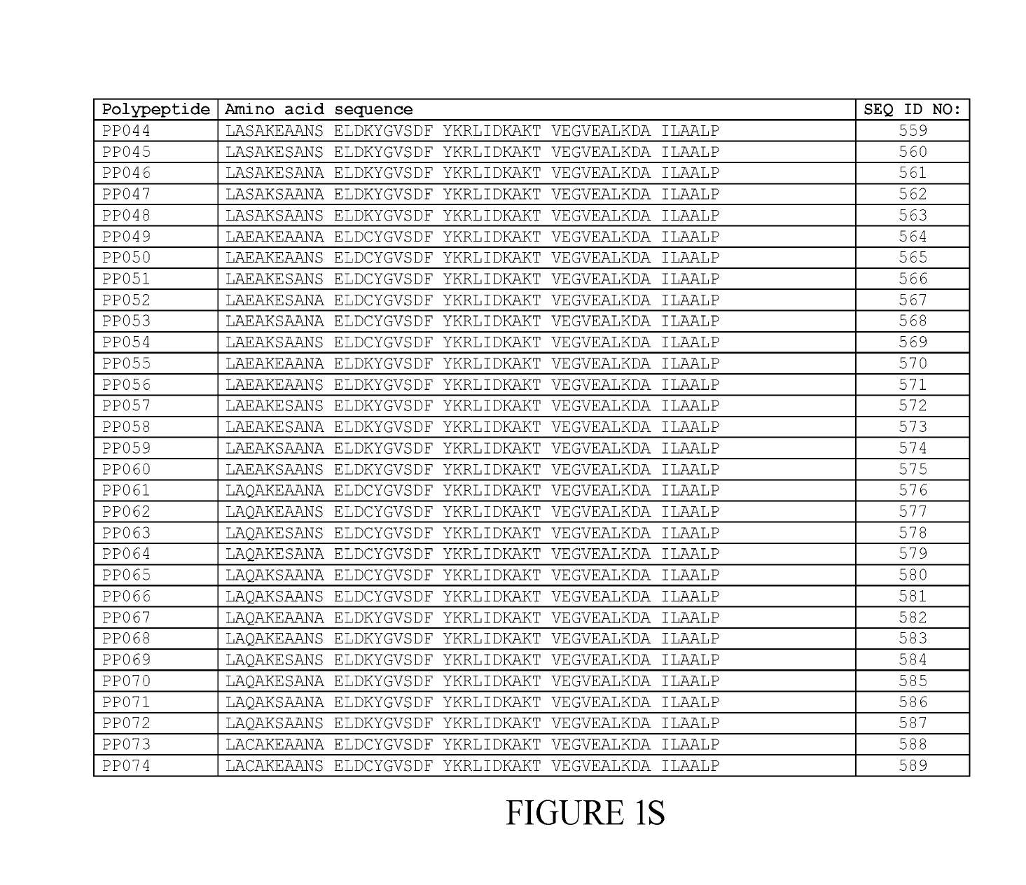

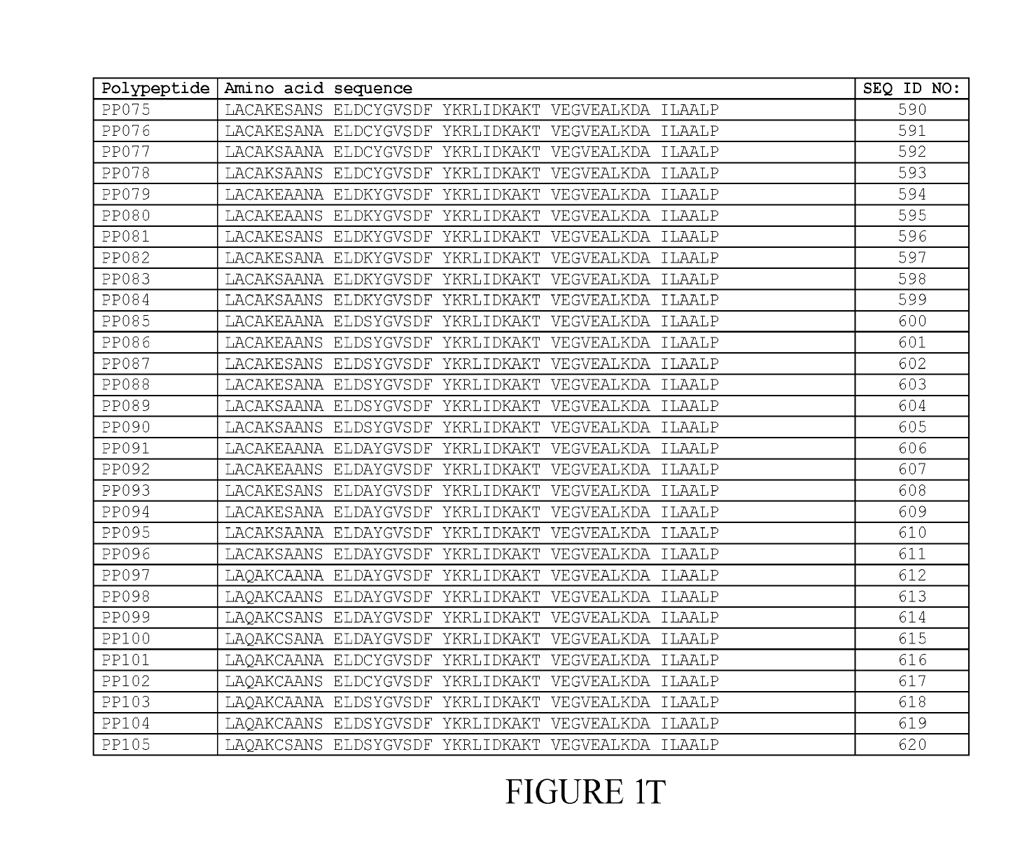

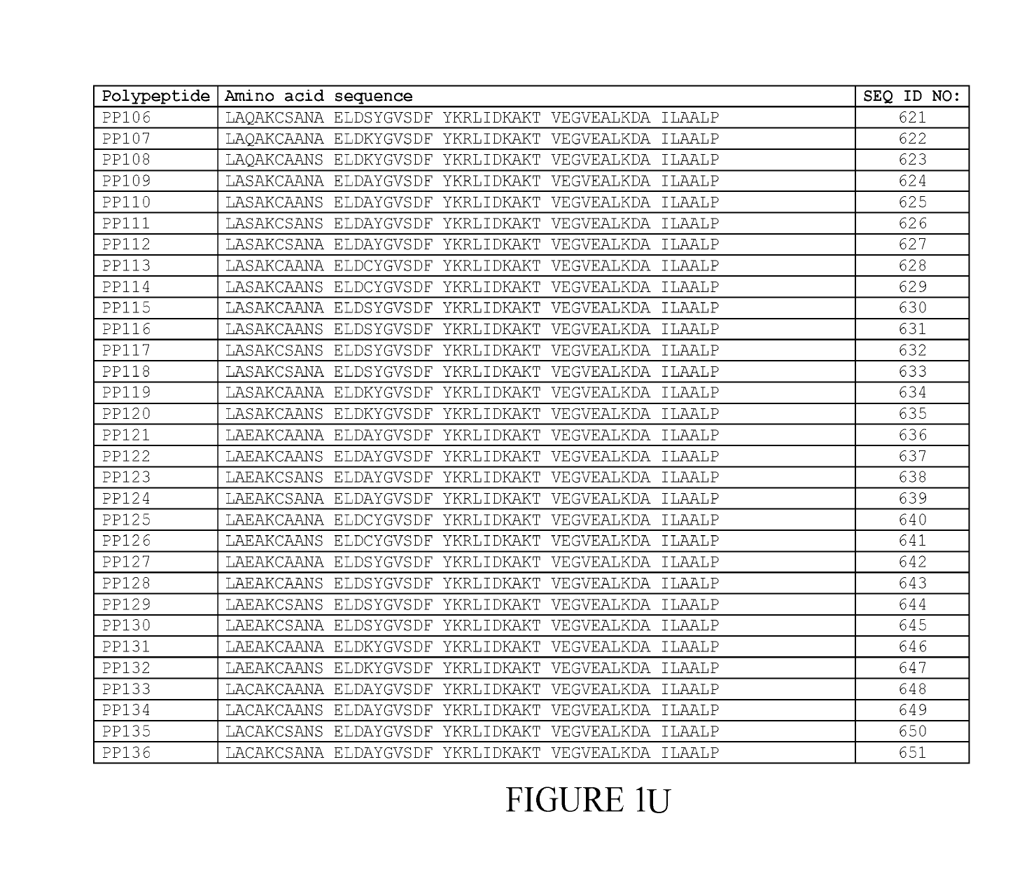

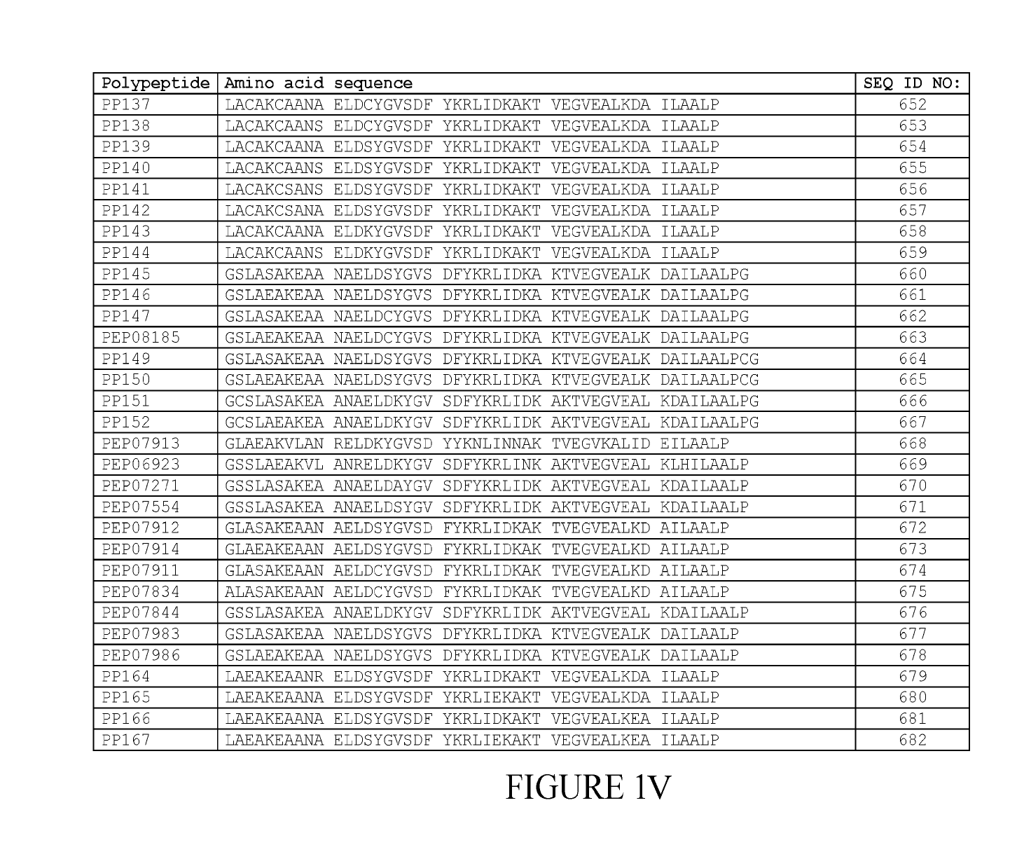

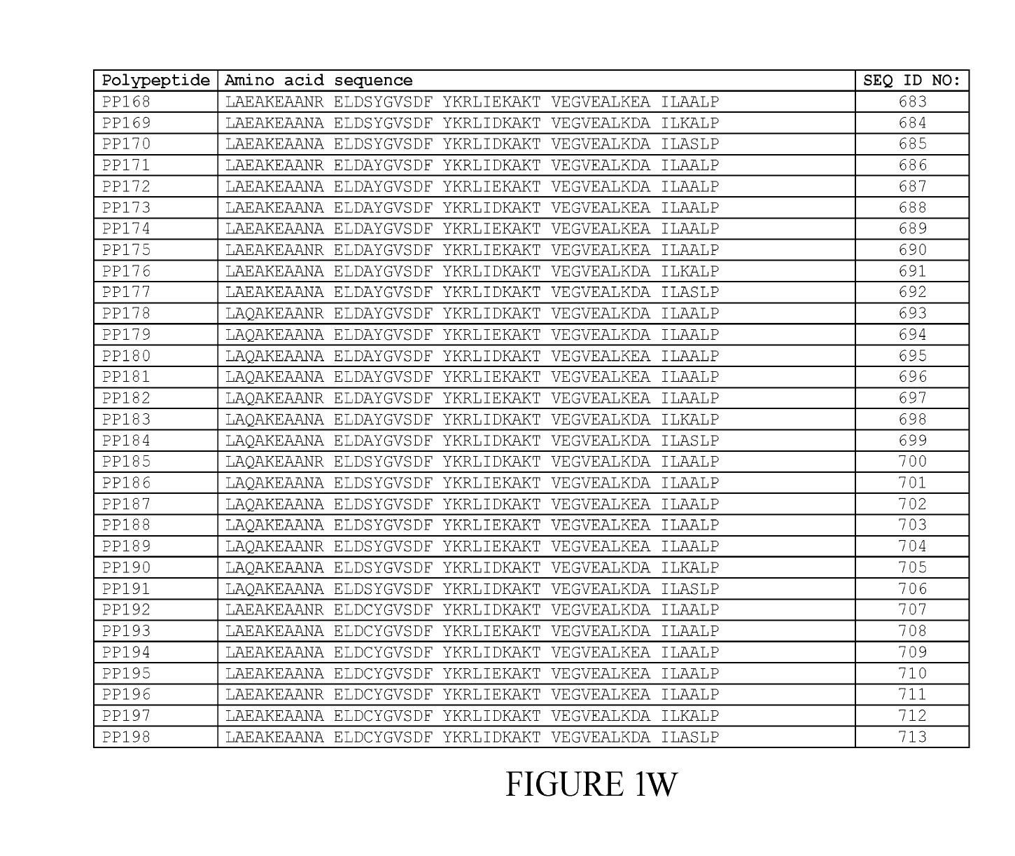

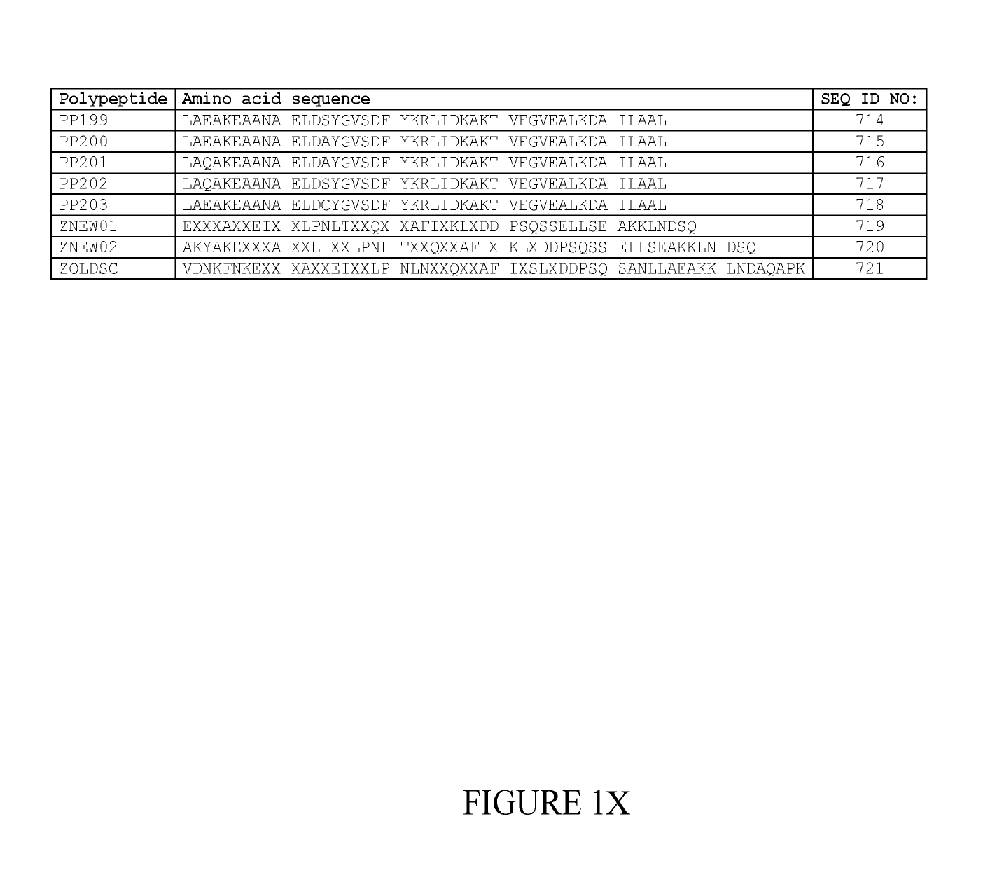

The purity of the produced polypeptides PEP03973, PEP10986 and PEP04419 was estimated to exceed 98% as assessed by SDS-PAGE analysis (FIG. 1). The prepared concentrations as determined by absorbance measurements at 280 nm and the correct molecular weights verified by LC/MS-analyses are summarized in Table 1.

TABLE 1
Prepared concentrations and molecular
weights of purified polypeptides

| | | Theoretical | Determined |
|---|---|---|---|
| Polypeptide | Concentration | molecular weight | molecular weight |
| variant | (mg/ml) | (Da) | (Da) |
| PEP03973 | 39.5 | 12421.9 | 12420.9 ± 1.3 |
| PEP10986 | 55.2 | 12500.8 | 12499.9 ± 1.3 |
| PEP04419 | 109.7 | 7556.3 | 7555.7 ± 0.8 |

### Example 2

### Pharmacokinetic Analysis of Orally Administered Polypeptide Variants in Mice

### Materials and Methods

### Oral Administration:

The polypeptide variants PEP03973, PEP10986 and PEP04419, prepared as described in Example 1, were administered orally at a dose of 65 μmol/kg, 92 μmol/kg and 298 μmol/kg body weight, respectively, to fasted male NMRI mice (Charles River, Germany) by gavage at time-point zero. Food was re-introduced approximately 30 minutes after administration. Serum samples were taken by cardiac puncture of anesthetized mice at 1, 3, 8, 24, 48 and 72 hours after administration of PEP03973 and PEP10986, and at 15, 30, 60, 180 and 480 minutes after administration of PEP04419. Three mice were sacrificed at each time-point. The concentration of respective protein in serum was measured by sandwich ELISA assays specific for each polypeptide variant.

### ELISA Assay for PEP03973:

ELISA plates (Greiner 96 well half area plates, cat no 675074) were coated over night at 4° C. with 1 μg/ml (50 μl/well) of in-house produced goat anti-protein Z immunoglobulins (Igs) in carbonate buffer (Sigma, cat no C3041). The next day the plates were blocked with PBS (2.68 mM KCl, 0.47 mM KH₂PO₄, 137 mM NaCl, 8.1 mM Na₂HPO₄, pH 7.4)+0.5% casein (Sigma, cat no C8654), PBSC, for 1.5 h. Serum samples and purified PEP03973 used as standard were titrated in duplicates in a 3-fold dilution series in PBSC in a total volume of 50 μl/well and incubated for another 1.5 h. Bound PEP03973 was detected by addition of 50 μl/well of in-house produced rabbit anti-protein Z/ABDwt Ig diluted to 1 μg/ml in PBSC, followed by 50 μl/well of DELFIA Eu-N1-anti-rabbit IgG (Perkin Elmer cat no AD0105) diluted to 20 ng/ml in PBSC. The antibody incubation periods were 1.5 and 1 hour, respectively, with washes after each step. Subsequently, 50 μl/well of Enhancement Solution (Perkin Elmer cat 4001-0010) was added and time resolved fluorescence (TRF) was read in an ELISA reader (Victor³, Perkin Elmer) after 5 minutes of gentle agitation. The concentration of PEP03973 in serum samples was calculated from the PEP03973 standard curve using nonlinear regression (Graph Pad Prism5).

### ELISA Assay for PEP10986:

The concentration of PEP10986 in serum samples was determined by an ELISA assay similar to that described above for PEP03973, but coating was performed with 8 μg/ml of in-house produced goat anti-protein Z Ig, and the first detection step was performed using in-house produced rabbit Ig specific for the albumin binding domain specified by SEQ ID NO:2 above, at a concentration of 2 μg/ml.

ELISA assay for PEP04419:

ELISA plates (Costar 96 well half area plates, cat no 3690) were coated overnight at 4° C. with 2 μg/ml (50 μl/well) of goat Ig against protein Z, in carbonate buffer. The next day, the plates were blocked with PBSC for 1.5 hours. Serum samples and purified PEP04419 used as standard were titrated in duplicates in a 2-fold dilution series in PBSC in a total volume of 50 μl/well and incubated for another 1.5 hours. The plate was washed four times in PBS+0.05% Tween (PBST). Bound PEP04419 was detected with 50 μl/well of rabbit anti-protein Z Ig (0.125 μg/ml in PBSC) followed by 50 μl/well of anti-rabbit IgG-HRP (Dako, cat no P0448) diluted 1:10000 in PBSC. Each antibody was incubated for 1 hour with washes after each step. After the final wash, the reaction was developed with 50 μl/well of TMB Immunopure (Thermo Scientific, cat no 34021) and stopped by the addition of 50 μl/well of H₂SO₄. The absorbance at 450 nm was read in an ELISA reader (Victor³, Perkin Elmer). The concentration of PEP04419 in serum samples was calculated from the PEP04419 standard curve using nonlinear regression (Graph Pad Prism5).

### Results

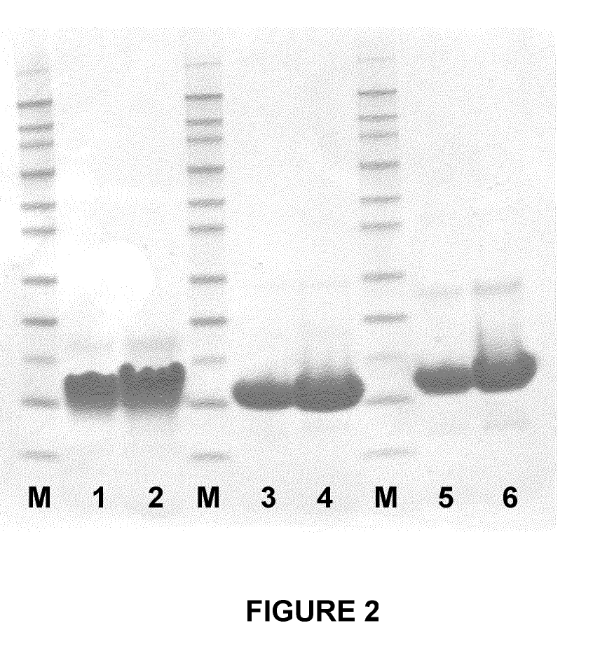

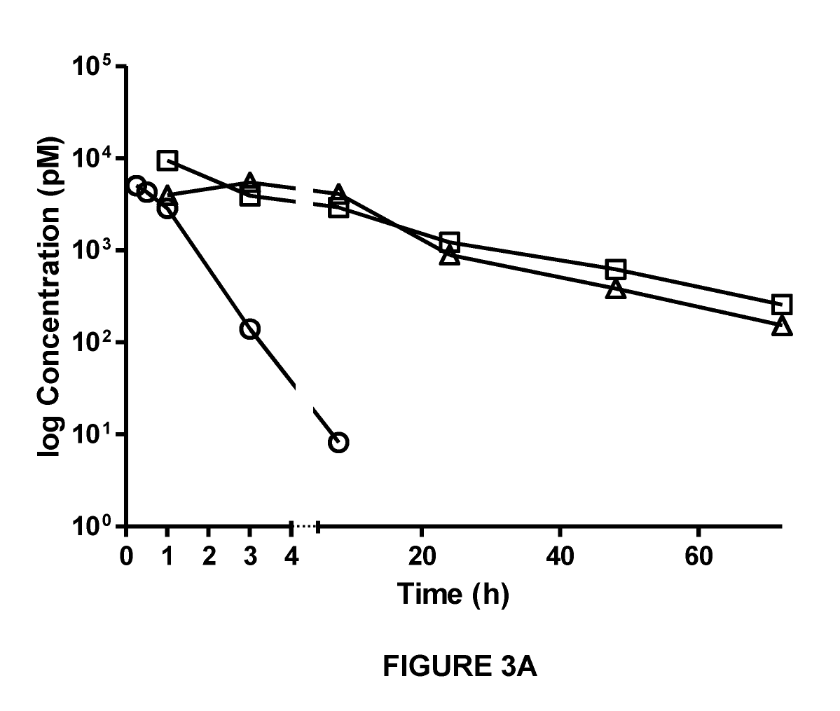

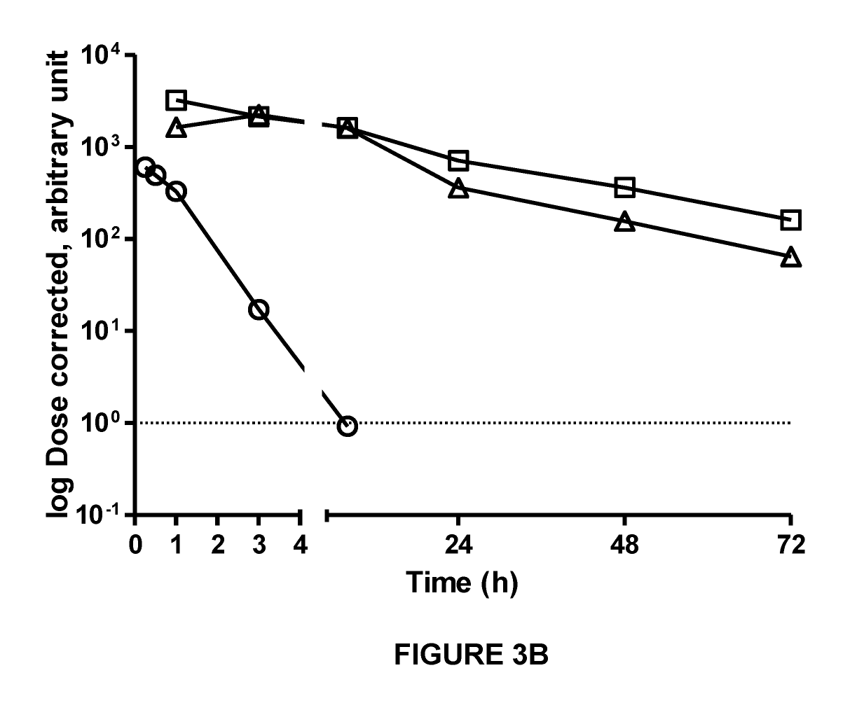

As can be seen in FIG. 2, all three administered polypeptides were taken up after administration via the oral route. Surprisingly, the polypeptides comprising an albumin binding moiety, i.e. PEP03973 and PEP10986, were taken up at least as well as the polypeptide without albumin binding moiety, PEP04419, despite being nearly twice the size. Furthermore, the polypeptides comprising an albumin binding moiety showed a much longer residence time compared to the polypeptide without albumin binding moiety, which could not be detected in samples taken 8 hours after administration (see FIGS. 3A and 3B). The area under curve (AUC), was 106, 96 and 7 nMh for PEP03973, PEP10986 and PEP044194, respectively. The expected pharmacokinetic profiles indicate that PEP03973 and PEP10986 retain their albumin binding capability upon oral administration.

### Example 3

### Pharmacokinetic Analysis of Oral and Duodenal Administration of PEP10896 in Rat

### Materials and Methods

### Oral Administration:

The polypeptide PEP10986, prepared as described in Example 1, was administered orally by gavage at time-point zero at a dose of 67 μmol/kg body weight to fasted male Sprague Dawley rats (Charles River, Germany). Food was re-introduced approximately 30 min after administration. Blood samples were taken under isoflurane anesthesia at 1, 3, 8, 24, 72, 120 and 168 hours after administration, and serum was prepared by standard procedures. The concentration of PEP10896 in serum was measured by the sandwich ELISA assay described in Example 2.

### Duodenal Administration:

An indwelling catheter (IITC Life Science, cat no S2C6) was inserted surgically into the duodenum 10-15 mm from its origin, and tunneled subcutaneously to the back of anaesthetized male Sprague Dawley rats (Charles River). After a recovery period of 3-5 days, the polypeptide PEP10986, prepared as described in Example 1, was administered directly to the duodenum at a dose of 67 μmol/kg body weight to fasted rats at time-point zero. Food was re-introduced approximately 30 minutes after administration. Blood samples were taken under isoflurane anesthesia at 1, 3, 8, 24, 72, 120 and 168 hours after administration of PEP10986, and serum was prepared by standard procedures. The concentration of PEP10896 in serum was measured by the sandwich ELISA assay described in Example 2.

### Results

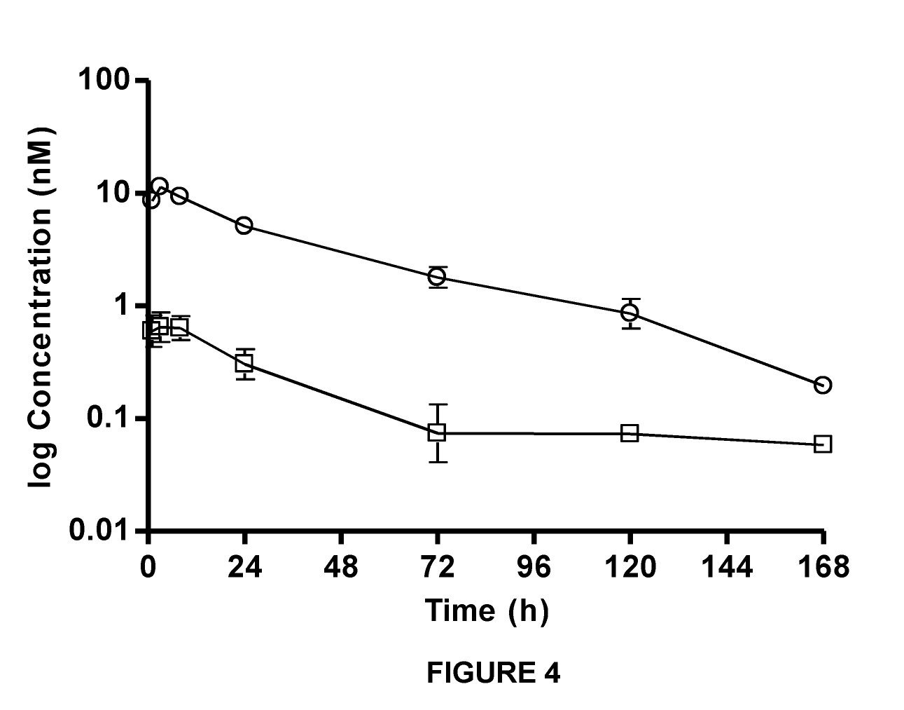

The pharmacokinetic profiles of PEP10896 obtained after oral and intraduodenal administration are presented in FIG. 4. The results showed that PEP10896 was taken up via the oral route in rat after both oral gavage and intraduodenal administration. Duodenal administration contributed to increased intestinal uptake as indicated by a higher Cmax (3 hours) concentration compared to oral gavage (11.3 versus 0.71 nM). At least 15 times higher AUC was obtained as a result of improved uptake. The prolonged pharmacokinetic profiles, with half-lives similar to albumin, indicated that PEP10986 retained the albumin binding capacity after oral uptake, whether by gavage or intraduodenal administration.

### Example 4

### Pharmacokinetic Analysis Repeated Duodenal Administration of PEP10896 in Rat

### Materials and Methods

### Repeated Duodenal Administration:

polypeptide variant PEP10986, prepared as described in Example 1, was administered directly into the duodenum of fasted rats as described in Example 3. PEP10986 was administered three times at a dose of 67 μmol/kg body weight at time point zero, 2 and 24 hours. Food was re-introduced approximately 30 minutes after each administration. Blood samples were taken under isoflurane anesthesia at 1, 3, 5, 24, 27, 96, 144 and 192 hours after administration of PEP10986, and serum was prepared according to standard procedures. The concentration of PEP10896 in serum was measured by the sandwich ELISA assay described in Example 2.

### Results

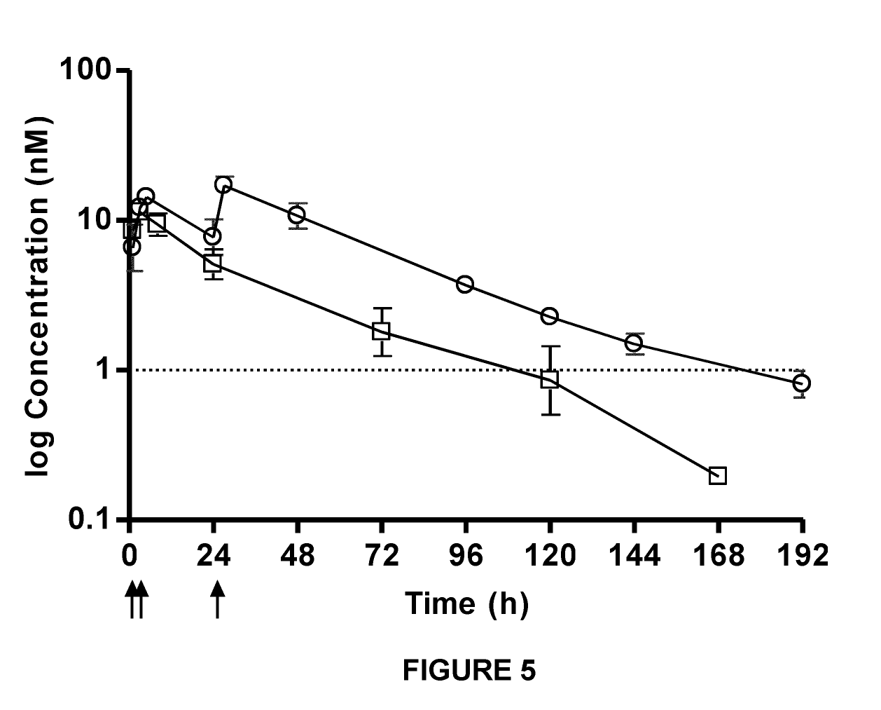

The pharmacokinetic profile of PEP10896 obtained after repeated intraduodenal administration in shown in FIG. 5. The result from duodenal administration described in Example 3 is included in FIG. 5 to emphasize the difference between single and repeated administrations. Repeated administration boosted the concentration of polypeptide PEP10896 in rat serum and prolonged the pharmacokinetic profile, indicating that PEP10986 retained the albumin binding capability after oral uptake. The concentration increased more than two times after the third administration, indicating that higher serum levels are possible to obtain by repeated administration.

### Itemized Listing of Embodiments

1. Compound for use in treatment via oral administration, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment.

2. Compound for use according to item 1, in which said moiety (I) comprises a component selected from the group consisting of human endogenous enzymes, hormones, growth factors, chemokines, cytokines, blood clotting and complement factors, innate immune defense and regulatory peptides, for example selected from the group consisting of insulin, insulin analogs, IL-2, IL-5, GLP-1, BNP, IL 1-RA, KGF, Stemgen®, GH, G-CSF, CTLA-4, myostatin, Factor VII, Factor VIII and Factor IX, and derivatives of anyone thereof.

3. Compound for use according to item 1, in which said moiety (I) comprises a non-human biologically active protein, selected from the group consisting of modulins, bacterial toxins, hormones, innate immune defense and regulatory peptides, enzymes and activating proteins.

4. Compound for use according to item 1, in which said moiety (I) comprises a binding polypeptide capable of selective interaction with a target molecule.

5. Compound for use according to item 4, in which the binding polypeptide is selected from the group consisting of antibodies and fragments and domains thereof substantially retaining antibody binding activity; microbodies, maxybodies, avimers and other small disulfide-bonded proteins; and binding proteins derived from a scaffold selected from the group consisting of staphylococcal protein A and domains thereof, other three helix domains, lipocalins, ankyrin repeat domains, cellulose binding domains, γ crystallines, green fluorescent protein, human cytotoxic T lymphocyte-associated antigen 4, protease inhibitors such as Kunitz domains, PDZ domains, SH3 domains, peptide aptamers, staphylococcal nuclease, tendamistats, fibronectin type III domain, transferrin, zinc fingers and conotoxins.

6. Compound for use according to item 5, in which said binding polypeptide comprises a variant of protein Z derived from domain B of staphylococcal protein A, which variant comprises a scaffold amino acid sequence selected from SEQ ID NO:719, SEQ ID NO:720 and SEQ ID NO:721, wherein X denotes any amino acid residue.

7. Compound for use according to any one of items 4-6, in which said target molecule is selected from the group consisting of tumor-related or other cell surface related antigens, such as CD14, CD19, CD20, CD22, CD30, CD33, CD37, CD40, CD52, CD56, CD70, CD138, cMet, HER1, HER2, HER3, HER4, CAIX, CEA, IL-2 receptor, IGF1R, VEGFR2, MUC1, PDGFR-beta, PSMA, TAG-72, FOLR1, mesothelin, CA6, GPNMB, integrins and ephA2; cytokines such as TNF-α, IL-1α, IL-1β, IL-1Ra, IL-5, IL-6, IL-13, IL-17A, IL-18, IL-23, IL-36, G-CSF, GM-CSF, and their receptors; chemokines such as IL-8, CCL-2 and CCL11, and their receptors; complement factors such as C3 and factor D; growth factors such as HGF and myostatin; hormones such as GH, insulin and somatostatin; peptides such as AR peptide of Alzheimer's disease; other disease-associated amyloid peptides; hypersensitivity mediators such as histamine and IgE; blood clotting factors, such as von Willebrand factor; and toxins, such as bacterial toxins and snake venoms.

8. Compound for use according to item 1, in which said moiety (I) comprises a non-proteinaceous component selected from the group consisting of a) cytotoxic agents, for example calicheamycin, auristatin, doxorubicin, maytansinoid, taxane, ecteinascidin, geldanamycin, methotrexate, camptothecin, cyclophosphamide, cyclosporine and their derivatives, and combinations thereof; and b) anti-inflammatory agents, for example non-steroidal anti-inflammatory drugs, cytokine suppressive anti-inflammatory drugs, corticosteroids, methotrexate, prednisone, cyclosporine, morroniside cinnamic acid, leflunomide and their derivatives, and combinations thereof.

9. Compound for use according to any preceding item, in which said moiety (II) comprises a naturally occurring GA domain or a derivative thereof.

10. Compound for use according to item 9, in which said moiety (II) comprises a GA domain selected from the group consisting of domain GA1, domain GA2 and domain GA3 of protein G from Streptococcus strain G148, and derivatives thereof.

11. Compound for use according to item 10, in which said moiety (II) comprises domain GA3 of protein G from Streptococcus strain G148 or a derivative thereof.

12. Compound for use according to item 11, in which said moiety (II) comprises domain GA3 of protein G from Streptococcus strain G148 having the amino acid sequence SEQ ID NO:515.

13. Compound for use according to any one of items 1-11, in which said moiety (II) comprises an albumin binding motif, which motif consists of the amino acid sequence:

GVSDX₅YKX₈X₉I X₁₁X₁₂AX₁₄TVEGVX₂₀ ALX₂₃X₂₄X₂₅I

wherein, independently of each other,
X₅ is selected from Y and F;
X₈ is selected from N, R and S;
X₉ is selected from V, I, L, M, F and Y;
X₁₁ is selected from N, S, E and D;
X₁₂ is selected from R, K and N;
X₁₄ is selected from K and R;
X₂₀ is selected from D, N, Q, E, H, S, R and K;
X₂₃ is selected from K, I and T;
X₂₄ is selected from A, S, T, G, H, L and D; and
X₂₅ is selected from H, E and D.

14. Compound for use according to item 13, wherein X₅ is Y.

15. Compound for use according to any one of items 13-14, wherein X₈ is selected from N and R, in particular R.

16. Compound for use according to any one of items 13-15, wherein X₉ is L.

17. Compound for use according to any one of items 13-16, wherein X₁₁ is selected from N and S, in particular N.

18. Compound for use according to any one of items 13-17, wherein X₁₂ is selected from R and K, is R, or is K.

19. Compound for use according to any one of items 13-18, wherein X₁₄ is K.

20. Compound for use according to any one of items 13-19, wherein X₂₀ is selected from D, N, Q, E, H, R and S, in particular E.

21. Compound for use according to any one of items 13-20, wherein X₂₃ is selected from K and I, in particular K.

22. Compound for use according to any one of items 13-21, wherein X₂₄ is selected from A, S, T, G, H and L, in particular L.

23. Compound for use according to item 22, wherein X₂₄ is L.

24. Compound for use according to item 23, wherein X₂₃X₂₄ is KL.

25. Compound for use according to item 23, wherein X₂₃X₂₄ is TL.

26. Compound for use according to item 22, wherein X₂₄ is selected from A, S, T, G and H.

27. Compound for use according to item 26, wherein X₂₃ is I.

28. Compound for use according to any one of items 13-27, wherein

X₂₅ is H.

29. Compound for use according to any one of items 13-28, in which said albumin binding motif consists of an amino acid sequence selected from SEQ ID NO:1-257, in particular selected from SEQ ID NO:2, SEQ ID NO:3, SEQ ID NO:9, SEQ ID NO:15, SEQ ID NO:25, SEQ ID NO:27, SEQ ID NO:46, SEQ ID NO:49, SEQ ID NO:53, SEQ ID NO:54, SEQ ID NO:55, SEQ ID NO:155, SEQ ID NO:239, SEQ ID NO:240, SEQ ID NO:241, SEQ ID NO:242, SEQ ID NO:243, SEQ ID NO:244 and SEQ ID NO:245, more in particular selected from SEQ ID NO:3, SEQ ID NO:53 and SEQ ID NO:239.

30. Compound for use according to any one of items 13-29, in which moiety (II) comprises the amino acid sequence:

LAEAKXₐX_{b}AX_{c}X_{d} ELXₑKY-[ABM]-LAALP

wherein
[ABM] is an albumin binding motif as defined in any one of items 13-29,
and, independently of each other,
Xₐ is selected from V and E;
X_{b} is selected from L, E and D;
X_{c} is selected from N, L and I;
X_{d} is selected from R and K; and
Xₑ is selected from D and K.

31. Compound for use according to item 30, wherein Xₐ is V.

32. Compound for use according to any one of items 30-31, wherein X_{b} is L.

33. Compound for use according to any one of items 30-32, wherein X_{c} is N.

34. Compound for use according to any one of items 30-33, wherein X_{d} is R.

35. Compound for use according to any one of items 30-34, wherein Xₑ is D.

36. Compound for use according to item 11, in which said moiety (II) comprises a derivative of domain GA3 of protein G from Streptococcus strain G148, which derivative comprises an amino acid sequence which fulfils one definition selected from the following:
i) it is selected from SEQ ID NO:258-514, in particular selected from SEQ ID NO:259, SEQ ID NO:260, SEQ ID NO:266, SEQ ID NO:272, SEQ ID NO:282, SEQ ID NO:284, SEQ ID NO:303, SEQ ID NO:306, SEQ ID NO:310, SEQ ID NO:311, SEQ ID NO:312, SEQ ID NO:412, SEQ ID NO:496, SEQ ID NO:497, SEQ ID NO:498, SEQ ID NO:499, SEQ ID NO:500, SEQ ID NO:501 and SEQ ID NO:502, more in particular selected from SEQ ID NO:260, SEQ ID NO:310 and SEQ ID NO:496;
ii) it is an amino acid sequence having 85% or greater identity to a sequence of (i).

37. Compound for use according to item 11, in which said moiety (II) comprises a derivative of domain GA3 of protein G from Streptococcus strain G148, which derivative comprises an amino acid sequence selected from

i) LAX₃AKX₆X₇ANX₁₀ ELDX₁₄YGVSDF YKRLIX₂₆KAKT
VEGVEALKX₃₉X₄₀ ILX₄₃X₄₄LP

wherein, independently of each other,
X₃ is selected from E, S, Q and C;
X₆ is selected from E, S and C;
X₇ is selected from A and S;
X₁₀ is selected from A, S and R;
X₁₄ is selected from A, S, C and K;
X₂₆ is selected from D and E;
X₃₉ is selected from D and E;
X₄₀ is selected from A and E;
X₄₃ is selected from A and K;
X₄₄ is selected from A, S and E;
L in position 45 is present or absent; and
P in position 46 is present or absent;
and
ii) an amino acid sequence which has at least 95% identity to the sequence defined in i).

38. Compound for use according to item 37, wherein X₆ is E.

39. Compound for use according to any one of items 37-38, wherein X₃ is S or is E.

40. Compound for use according to any one of items 37-39, wherein X₇ is A.

41. Compound for use according to any one of items 37-40, wherein X₁₄ is S or is C.

42. Compound for use according to any one of items 37-41, wherein X₁₀ is A or is S.

43. Compound for use according to any one of items 37-42, wherein X₂₆ is D or is E.

44. Compound for use according to any one of items 37-43, wherein X₃₉ is D or is E.

45. Compound for use according to any one of items 37-44, wherein X₄₀ is A.

46. Compound for use according to any one of items 37-45, wherein X₄₃ is A.

47. Compound for use according to any one of items 37-46, wherein X₄₄ is A or is S.

48. Compound for use according to any one of items 37-47, wherein L in position 45 is present.

49. Compound for use according to any one of items 37-48, wherein P in position 46 is present.

50. Compound for use according to item 37, in which the derivative comprises an amino acid sequence selected from the group consisting of SEQ ID NO:516-659 and SEQ ID NO:679-718, in particular selected from the group consisting of SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557, SEQ ID NO:564-565, SEQ ID NO:679-685 and SEQ ID NO:707-718, or selected from the group consisting of SEQ ID NO:516-659, in particular selected from any the group consisting of SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557 and SEQ ID NO:564-565.

51. Compound for use according to any one of items 37-50, further comprising one or more additional amino acid residues positioned at the N- and/or the C-terminal of the sequence defined in i).

52. Compound for use according to item 51, in which the derivative comprises an amino acid sequence selected from the group consisting of SEQ ID NO:660-665 and SEQ ID NO:677-678.

53. Compound for use according to any preceding item, in which moiety (II) binds to albumin such that the K_{D} of the interaction is at most 1×10⁻⁸ M, for example at most 1×10⁻⁹ M, for example at most 1×10⁻¹⁰ M, for example at most 1×10⁻¹¹ M, for example at most 1×10⁻¹² M.

54. Pharmaceutical composition for oral administration, comprising:

a) a compound, which comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment; and

b) at least one pharmaceutically acceptable excipient.

55. Pharmaceutical composition according to item 54, in which said compound is as defined in any one of items 2-53.

56. Pharmaceutical composition according to any one of items 54-55, which further comprises at least one component for increasing oral bioavailability of said therapeutic activity.

57. Pharmaceutical composition according to item 56, wherein said component is selected from the group consisting of protease inhibitors, absorbance enhancers, mucoadhesive polymers, formulation vehicles and any combination thereof.

58. Pharmaceutical composition according to any one of items 54-57, which is present in a form selected from solid forms, such as pills, tablets, capsules, powders or granules; semi-solid forms, such as pastes; and liquid forms, such as elixirs, solutions or suspensions.

59. Pharmaceutical composition according to any one of items 54-58, in a formulation designed for immediate, delayed or controlled release.

60. Pharmaceutical composition according to any one of items 54-59, formulated as enteric-coated capsules.

61. Method of treatment of a mammalian subject in need of such treatment, comprising oral administration of a compound, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,

with the proviso that moiety (I) is not selected from an exendin sequence, an exendin analog sequence, an exendin active fragment sequence or an exendin analog active fragment.

62. Method of treatment of a mammalian subject in need of such treatment, comprising oral administration of a pharmaceutical composition according to any one of items 54-60.

63. Method according to any one of items 61-62, in which said compound is as defined in any one of items 2-53.

1. A Compound for use in treatment via oral administration, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,
with the proviso that moiety (I) is not selected from an exendin sequence, an exendin analog sequence, an exendin active fragment sequence or an exendin analog active fragment.

2. The Compound for use according to claim 1, in which said moiety (I) comprises a binding polypeptide capable of selective interaction with a target molecule.

3. The Compound for use according to claim 2, in which the binding polypeptide is a variant of protein Z derived from domain B of staphylococcal protein A, which variant comprises a scaffold amino acid sequence selected from SEQ ID NO:719, SEQ ID NO:720 and SEQ ID NO:721, wherein X denotes any amino acid residue.

4. The Compound for use according to claim 3, in which said moiety (II) comprises domain GA3 of protein G from Streptococcus strain G148 having the amino acid sequence SEQ ID NO:515.

5. The Compound for use according to claim 1, in which said moiety (II) comprises an albumin binding motif, which motif consists of the amino acid sequence:
(SEQ ID NO: 722)
GVSDX₅YKX₈X₉I X₁₁X₁₂AX₁₄TVEGVX₂₀ ALX₂₃X₂₄X₂₅I
wherein, independently of each other,
X₅ is selected from Y and F;
X₈ is selected from N, R and S;
X₉ is selected from V, I, L, M, F and Y;
X₁₁ is selected from N, S, E and D;
X₁₂ is selected from R, K and N;
X₁₄ is selected from K and R;
X₂₀ is selected from D, N, Q, E, H, S, R and K;
X₂₃ is selected from K, I and T;
X₂₄ is selected from A, S, T, G, H, L and D; and
X₂₅ is selected from H, E and D.

6. The Compound for use according to claim 5, in which said albumin binding motif consists of an amino acid sequence selected from SEQ ID NO:1-257, in particular selected from SEQ ID NO:2, SEQ ID NO:3, SEQ ID NO:9, SEQ ID NO:15, SEQ ID NO:25, SEQ ID NO:27, SEQ ID NO:46, SEQ ID NO:49, SEQ ID NO:53, SEQ ID NO:54, SEQ ID NO:55, SEQ ID NO:155, SEQ ID NO:239, SEQ ID NO:240, SEQ ID NO:241, SEQ ID NO:242, SEQ ID NO:243, SEQ ID NO:244 and SEQ ID NO:245, more in particular selected from SEQ ID NO:3, SEQ ID NO:53 and SEQ ID NO:239.

7. The Compound for use according to claim 5, in which moiety (II) comprises the amino acid sequence:
(SEQ ID NO: 723)
LAEAKXₐX_{b}AX_{c}X_{d} ELXₑKY-[ABM]-LAALP
wherein
[ABM] is an albumin binding motif as defined in claim 5,
and, independently of each other,
Xₐ is selected from V and E;
X_{b} is selected from L, E and D;
X_{c} is selected from N, L and I;
X_{d} is selected from R and K; and
Xₑ is selected from D and K.

8. The Compound for use according to claim 3, in which said moiety (II) comprises a derivative of domain GA3 of protein G from Streptococcus strain G148, which derivative comprises an amino acid sequence which fulfils one definition selected from the following:
i) it is selected from SEQ ID NO:258-514, in particular selected from SEQ ID NO:259, SEQ ID NO:260, SEQ ID NO:266, SEQ ID NO:272, SEQ ID NO:282, SEQ ID NO:284, SEQ ID NO:303, SEQ ID NO:306, SEQ ID NO:310, SEQ ID NO:311, SEQ ID NO:312, SEQ ID NO:412, SEQ ID NO:496, SEQ ID NO:497, SEQ ID NO:498, SEQ ID NO:499, SEQ ID NO:500, SEQ ID NO:501 and SEQ ID NO:502, more in particular selected from SEQ ID NO:260, SEQ ID NO:310 and SEQ ID NO:496;
ii) it is an amino acid sequence having 85% or greater identity to a sequence of (i).

9. The Compound for use according to claim 3, in which said moiety (II) comprises a derivative of domain GA3 of protein G from Streptococcus strain G148, which derivative comprises an amino acid sequence selected from
(SEQ ID NO: 724)
i)LAX₃AKX₆X₇ANX₁₀ ELDX₁₄YGVSDF YKRLIX₂₆KAKT
VEGVEALKX₃₉X₄₀ ILX₄₃X₄₄LP
wherein, independently of each other,
X₃ is selected from E, S, Q and C;
X₆ is selected from E, S and C;
X₇ is selected from A and S;
X₁₀ is selected from A, S and R;
X₁₄ is selected from A, S, C and K;
X₂₆ is selected from D and E;
X₃₉ is selected from D and E;
X₄₀ is selected from A and E;
X₄₃ is selected from A and K;
X₄₄ is selected from A, S and E;
L in position 45 is present or absent; and
P in position 46 is present or absent;
and
ii) an amino acid sequence which has at least 95% identity to the sequence defined in i).

10. The Compound for use according to claim 9, in which the derivative comprises an amino acid sequence selected from the group consisting of SEQ ID NO:516-659 and SEQ ID NO:679-718, in particular selected from the group consisting of SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557, SEQ ID NO:564-565, SEQ ID NO:679-685 and SEQ ID NO:707-718, or selected from the group consisting of SEQ ID NO:516-659, in particular selected from any the group consisting of SEQ ID NO:519-520, SEQ ID NO:522-523, SEQ ID NO:525-526, SEQ ID NO:528-529, SEQ ID NO:531-532, SEQ ID NO:534-535, SEQ ID NO:537-538, SEQ ID NO:540-541, SEQ ID NO:543-544, SEQ ID NO:546-547, SEQ ID NO:549-550, SEQ ID NO:552-553, SEQ ID NO:556-557 and SEQ ID NO:564-565.

11. A Pharmaceutical composition for oral administration, comprising:
a) a compound, which comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,
with the proviso that moiety (I) is not selected from an exendin sequence, an exendin analog sequence, an exendin active fragment sequence or an exendin analog active fragment; and
b) at least one pharmaceutically acceptable excipient.

12. The Pharmaceutical composition according to claim 11, in which said compound is as defined in claim 2.

13. The Pharmaceutical composition according to claim 11, which further comprises at least one component for increasing oral bioavailability of said therapeutic activity.

14. The Pharmaceutical composition according to claim 11, formulated as enteric-coated capsules.

15. A Method of treatment of a mammalian subject in need of such treatment, comprising oral administration of a compound, which compound comprises
a moiety (I) which confers a desired therapeutic activity; and
an amino acid sequence corresponding to a moiety (II) which binds to albumin and comprises a naturally occurring, albumin binding protein selected from M1/Emm1, M3/Emm3, M12/Emm12, EmmL55/Emm55, Emm49/EmmL49, H, G, MAG, ZAG, PPL and PAB or an albumin binding domain, fragment or derivative of any one thereof,
with the proviso that moiety (I) is not selected from an exendin sequence, an exendin analog sequence, an exendin active fragment sequence or an exendin analog active fragment.

16. A Method of treatment of a mammalian subject in need of such treatment, comprising oral administration of a pharmaceutical composition according to claim 11.

17. The Method according to claim 15, in which said compound is as defined in claim 2.


SEQUENCE LISTING (724 sequences)

SEQ ID NO: 1 - protein, 26 aa, Artificial
  Engineered binding domain
GVSDFYKNLINRAKTVEGVHALIGHI

SEQ ID NO: 2 - protein, 26 aa, Artificial
  Engineered binding domain
GVSDFYKNVINRAKTVEGVHALIDHI

SEQ ID NO: 3 - protein, 26 aa, Artificial
  Engineered binding domain
GVSDYYKNIINRAKTVEGVRALKLHI

SEQ ID NO: 4 - protein, 26 aa, Artificial
  Engineered binding domain
GVSDFYKNVINRAKTVEGVSALIHEI

SEQ ID NO: 5 - protein, 26 aa, Artificial
  Engineered binding domain
GVSDFYKRLINRAKTVEGVNTLIADI

SEQ ID NO: 6 - protein, 26 aa, Artificial
  Engineered binding domain
GVSDFYKNLINRAKTVEGVNTLIADI

SEQ ID NO: 7 - protein, 26 aa, Artificial
  Engineered binding domain
GVSDYYKNLINRAKTVEGVNSLISHI

SEQ ID NO: 8 - protein, 26 aa, Artificial
  Engineered binding domain
GVSDFYKRLINRAKTVGGVQSLISEI

SEQ ID NO: 9 - protein, 26 aa, Artificial
  Engineered binding domain
GVSDFYKNLINRAKTVEGVSSLKGHI

SEQ ID NO: 10 - protein, 26 aa, Artificial
  Engineered binding domain
GVSDFYKNVINRAKTVEGVDSLIAEI

SEQ ID NO: 11 - protein, 26 aa, Artificial
  Engineered binding domain
GVSDFYKNLINRARTVEGVQTLISDI

SEQ ID NO: 12 - protein, 26 aa, Artificial
  Engineered binding domain
GVSDFYKKFINKAKTVEGVETLISEI

SEQ ID NO: 13 - protein, 26 aa, Artificial
  Engineered binding domain
GVSDFYKSLINRAKTVEGVHSLTDEI

SEQ ID NO: 14 - protein, 26 aa, Artificial
  Engineered binding domain
GVSDYYKNVINKAKTVEGVSSLTAEI

SEQ ID NO: 15 - protein, 26 aa, Artificial
  Engineered binding domain
GVSDFYKSLINRAKTVEGVDALTSHI

SEQ ID NO: 16 - protein, 26 aa, Artificial
  Engineered binding domain
GVSDFYKNLINKAKTVEGVSTLIHDI

SEQ ID NO: 17 - protein, 26 aa, Artificial
  Engineered binding domain
GVSDFYKNLINRAKTVEGVSTLIHDI

SEQ ID NO: 18 - protein, 26 aa, Artificial
  Engineered binding domain
GVSDFYKNLINRAKTVEGVQALISEI

SEQ ID NO: 19 - protein, 26 aa, Artificial
  Engineered binding domain
GVSDYYKSLINKAKTVEGVDSLIVHI

SEQ ID NO: 20 - protein, 26 aa, Artificial
  Engineered binding domain
GVSDFYKNLINRAKTVEGVQSLITEI

SEQ ID NO: 21 - protein, 26 aa, Artificial
  Engineered binding domain
GVSDYYKNLINRAKTVEGVDALITHI

SEQ ID NO: 22 - protein, 26 aa, Artificial
  Engineered binding domain
GVSDFYKSMINRAKTVEGVDSLITHI

SEQ ID NO: 23 - protein, 26 aa, Artificial
  Engineered binding domain
GVSDFYKNLINRAKTVEGVTTLTTDI

SEQ ID NO: 24 - protein, 26 aa, Artificial
  Engineered binding domain
GVSDFYKNLINRAKTVEGVESLIDHI

SEQ ID NO: 25 - protein, 26 aa, Artificial
  Engineered binding domain
GVSDFYKSYINRAKTVEGVHTLIGHI

SEQ ID NO: 26 - protein, 26 aa, Artificial
  Engineered binding domain
GVSDFYKNLINRAKTVEGVQTLISDI

SEQ ID NO: 27 - protein, 26 aa, Artificial
  Engineered binding domain
GVSDFYKNLINRAKTVEGVNSLTSHI

SEQ ID NO: 28 - protein, 26 aa, Artificial
  Engineered binding domain
GVSDFYKNLINRAKTVEGVNTLIHDI

SEQ ID NO: 29 - protein, 26 aa, Artificial
  Engineered binding domain
GVSDFYKNLINRAKTVEGVESLIGEI

SEQ ID NO: 30 - protein, 26 aa, Artificial
  Engineered binding domain
GVSDFYKNLINKAKTVEGVHTLIHDI

SEQ ID NO: 31 - protein, 26 aa, Artificial
  Engineered binding domain
GVSDYYKNLINKAKTVEGVSALKMHI

SEQ ID NO: 32 - protein, 26 aa, Artificial
  Engineered binding domain
GVSDFYKNLINKAKTVEGVDALIVHI

SEQ ID NO: 33 - protein, 26 aa, Artificial
  Engineered binding domain
GVSDYYKRLINRAKTVEGVHALIAEI

SEQ ID NO: 34 - protein, 26 aa, Artificial
  Engineered binding domain
GVSDYYKNLINRARTVEGVDTLIHDI

SEQ ID NO: 35 - protein, 26 aa, Artificial
  Engineered binding domain
GVSDFYKKVINRARTVEGVQALIADI

SEQ ID NO: 36 - protein, 26 aa, Artificial
  Engineered binding domain
GVSDFYKNLINRAKTVEGVESLIADI

SEQ ID NO: 37 - protein, 26 aa, Artificial
  Engineered binding domain
GVSDYYKNLINKAKTVEGVDALIAHI

SEQ ID NO: 38 - protein, 26 aa, Artificial
  Engineered binding domain
GVSDYYKNLINRAKTVEGVESLITHI

SEQ ID NO: 39 - protein, 26 aa, Artificial
  Engineered binding domain
GVSDFYKNLINRARTVEGVDSLIVEI

SEQ ID NO: 40 - protein, 26 aa, Artificial
  Engineered binding domain
GVSDFYKNVINRAKTVEGVSALIREI

SEQ ID NO: 41 - protein, 26 aa, Artificial
  Engineered binding domain
GVSDFYKNLINRAKTVEGVNALISDI

SEQ ID NO: 42 - protein, 26 aa, Artificial
  Engineered binding domain
GVSDFYKNLINRAKTVEGVSALIQEI

SEQ ID NO: 43 - protein, 26 aa, Artificial
  Engineered binding domain
GVSDFYKNLINRAKTVEGVQSLIDHI

SEQ ID NO: 44 - protein, 26 aa, Artificial
  Engineered binding domain
GVSDFYKNLINRAKTVEGVDALICHI

SEQ ID NO: 45 - protein, 26 aa, Artificial
  Engineered binding domain
GVSDYYKRLINKAKTVEGVNALITHI

SEQ ID NO: 46 - protein, 26 aa, Artificial
  Engineered binding domain
GVSDFYKNVINKAKTVEGVEALIADI

SEQ ID NO: 47 - protein, 26 aa, Artificial
  Engineered binding domain
GVSDFYKNLINRAKTVEGVETLIRDI

SEQ ID NO: 48 - protein, 26 aa, Artificial
  Engineered binding domain
GVSDFYKNLINRARTVEGVQTLITDI

SEQ ID NO: 49 - protein, 26 aa, Artificial
  Engineered binding domain
GVSDFYKRLINKAKTVEGVNALTHHI

SEQ ID NO: 50 - protein, 26 aa, Artificial
  Engineered binding domain
GVSDYYKNLINRAKTVEGVQALIAHI

SEQ ID NO: 51 - protein, 26 aa, Artificial
  Engineered binding domain
GVSDFYKNVINRAKTVEGVNSLINHI

SEQ ID NO: 52 - protein, 26 aa, Artificial
  Engineered binding domain
GVSDFYKSLINRARTVEGVDSLIRHI

SEQ ID NO: 53 - protein, 26 aa, Artificial
  Engineered binding domain
GVSDYYKNLINKAKTVEGVEALTLHI

SEQ ID NO: 54 - protein, 26 aa, Artificial
  Engineered binding domain
GVSDFYKNLINRAKTVEGVDALIAHI

SEQ ID NO: 55 - protein, 26 aa, Artificial
  Engineered binding domain
GVSDYYKNLINKAKTVEGVQALIAHI

SEQ ID NO: 56 - protein, 26 aa, Artificial
  Engineered binding domain
GVSDFYKRLINRAKTVEGVHALIGHI

SEQ ID NO: 57 - protein, 26 aa, Artificial
  Engineered binding domain
GVSDFYKRVINRAKTVEGVHALIDHI

SEQ ID NO: 58 - protein, 26 aa, Artificial
  Engineered binding domain
GVSDYYKRIINRAKTVEGVRALKLHI

SEQ ID NO: 59 - protein, 26 aa, Artificial
  Engineered binding domain
GVSDFYKRVINRAKTVEGVSALIHEI

SEQ ID NO: 60 - protein, 26 aa, Artificial
  Engineered binding domain
GVSDFYKRLINRAKTVEGVNTLIADI

SEQ ID NO: 61 - protein, 26 aa, Artificial
  Engineered binding domain
GVSDYYKRLINRAKTVEGVNSLISHI

SEQ ID NO: 62 - protein, 26 aa, Artificial
  Engineered binding domain
GVSDFYKRLINRAKTVEGVSSLKGHI

SEQ ID NO: 63 - protein, 26 aa, Artificial
  Engineered binding domain
GVSDFYKRVINRAKTVEGVDSLIAEI

SEQ ID NO: 64 - protein, 26 aa, Artificial
  Engineered binding domain
GVSDFYKRLINRARTVEGVQTLISDI

SEQ ID NO: 65 - protein, 26 aa, Artificial
  Engineered binding domain
GVSDFYKRFINKAKTVEGVETLISEI

SEQ ID NO: 66 - protein, 26 aa, Artificial
  Engineered binding domain
GVSDFYKRLINRAKTVEGVHSLTDEI

SEQ ID NO: 67 - protein, 26 aa, Artificial
  Engineered binding domain
GVSDYYKRVINKAKTVEGVSSLTAEI

SEQ ID NO: 68 - protein, 26 aa, Artificial
  Engineered binding domain
GVSDFYKRLINRAKTVEGVDALTSHI

SEQ ID NO: 69 - protein, 26 aa, Artificial
  Engineered binding domain
GVSDFYKRLINKAKTVEGVSTLIHDI

SEQ ID NO: 70 - protein, 26 aa, Artificial
  Engineered binding domain
GVSDFYKRLINRAKTVEGVSTLIHDI

SEQ ID NO: 71 - protein, 26 aa, Artificial
  Engineered binding domain
GVSDFYKRLINRAKTVEGVQALISEI

SEQ ID NO: 72 - protein, 26 aa, Artificial
  Engineered binding domain
GVSDYYKRLINKAKTVEGVDSLIVHI

SEQ ID NO: 73 - protein, 26 aa, Artificial
  Engineered binding domain
GVSDFYKRLINRAKTVEGVQSLITEI

SEQ ID NO: 74 - protein, 26 aa, Artificial
  Engineered binding domain
GVSDYYKRLINRAKTVEGVDALITHI

SEQ ID NO: 75 - protein, 26 aa, Artificial
  Engineered binding domain
GVSDFYKRMINRAKTVEGVDSLITHI

SEQ ID NO: 76 - protein, 26 aa, Artificial
  Engineered binding domain
GVSDFYKRLINRAKTVEGVTTLTTDI

SEQ ID NO: 77 - protein, 26 aa, Artificial
  Engineered binding domain
GVSDFYKRLINRAKTVEGVESLIDHI

SEQ ID NO: 78 - protein, 26 aa, Artificial
  Engineered binding domain
GVSDFYKRYINRAKTVEGVHTLIGHI

SEQ ID NO: 79 - protein, 26 aa, Artificial
  Engineered binding domain
GVSDFYKRLINRAKTVEGVQTLISDI

SEQ ID NO: 80 - protein, 26 aa, Artificial
  Engineered binding domain
GVSDFYKRLINRAKTVEGVNSLTSHI

SEQ ID NO: 81 - protein, 26 aa, Artificial
  Engineered binding domain
GVSDFYKRLINRAKTVEGVNTLIHDI

SEQ ID NO: 82 - protein, 26 aa, Artificial
  Engineered binding domain
GVSDFYKRLINRAKTVEGVESLIGEI

SEQ ID NO: 83 - protein, 26 aa, Artificial
  Engineered binding domain
GVSDFYKRLINKAKTVEGVHTLIHDI

SEQ ID NO: 84 - protein, 26 aa, Artificial
  Engineered binding domain
GVSDYYKRLINKAKTVEGVSALKMHI

SEQ ID NO: 85 - protein, 26 aa, Artificial
  Engineered binding domain
GVSDFYKRLINKAKTVEGVDALIVHI

SEQ ID NO: 86 - protein, 26 aa, Artificial
  Engineered binding domain
GVSDYYKRLINRARTVEGVDTLIHDI

SEQ ID NO: 87 - protein, 26 aa, Artificial
  Engineered binding domain
GVSDFYKRVINRARTVEGVQALIADI

SEQ ID NO: 88 - protein, 26 aa, Artificial
  Engineered binding domain
GVSDFYKRLINRAKTVEGVESLIADI

SEQ ID NO: 89 - protein, 26 aa, Artificial
  Engineered binding domain
GVSDYYKRLINKAKTVEGVDALIAHI

SEQ ID NO: 90 - protein, 26 aa, Artificial
  Engineered binding domain
GVSDYYKRLINRAKTVEGVESLITHI

SEQ ID NO: 91 - protein, 26 aa, Artificial
  Engineered binding domain
GVSDFYKRLINRARTVEGVDSLIVEI

SEQ ID NO: 92 - protein, 26 aa, Artificial
  Engineered binding domain
GVSDFYKRVINRAKTVEGVSALIREI

SEQ ID NO: 93 - protein, 26 aa, Artificial
  Engineered binding domain
GVSDFYKRLINRAKTVEGVNALISDI

SEQ ID NO: 94 - protein, 26 aa, Artificial
  Engineered binding domain
GVSDFYKRLINRAKTVEGVSALIQEI

SEQ ID NO: 95 - protein, 26 aa, Artificial
  Engineered binding domain
GVSDFYKRLINRAKTVEGVQSLIDHI

SEQ ID NO: 96 - protein, 26 aa, Artificial
  Engineered binding domain
GVSDFYKRLINRAKTVEGVDALICHI

SEQ ID NO: 97 - protein, 26 aa, Artificial
  Engineered binding domain
GVSDFYKRVINKAKTVEGVEALIADI

SEQ ID NO: 98 - protein, 26 aa, Artificial
  Engineered binding domain
GVSDFYKRLINRAKTVEGVETLIRDI

SEQ ID NO: 99 - protein, 26 aa, Artificial
  Engineered binding domain
GVSDFYKRLINRARTVEGVQTLITDI

SEQ ID NO: 100 - protein, 26 aa, Artificial
  Engineered binding domain
GVSDYYKRLINRAKTVEGVQALIAHI

SEQ ID NO: 101 - protein, 26 aa, Artificial
  Engineered binding domain
GVSDFYKRVINRAKTVEGVNSLINHI

SEQ ID NO: 102 - protein, 26 aa, Artificial
  Engineered binding domain
GVSDFYKRLINRARTVEGVDSLIRHI

SEQ ID NO: 103 - protein, 26 aa, Artificial
  Engineered binding domain
GVSDYYKRLINKAKTVEGVEALTLHI

SEQ ID NO: 104 - protein, 26 aa, Artificial
  Engineered binding domain
GVSDFYKRLINRAKTVEGVDALIAHI

SEQ ID NO: 105 - protein, 26 aa, Artificial
  Engineered binding domain
GVSDYYKRLINKAKTVEGVQALIAHI

SEQ ID NO: 106 - protein, 26 aa, Artificial
  Engineered binding domain
GVSDFYKNLINRAKTVEGVHALKGHI

SEQ ID NO: 107 - protein, 26 aa, Artificial
  Engineered binding domain
GVSDFYKNVINRAKTVEGVHALKDHI

SEQ ID NO: 108 - protein, 26 aa, Artificial
  Engineered binding domain
GVSDFYKNVINRAKTVEGVSALKHEI

SEQ ID NO: 109 - protein, 26 aa, Artificial
  Engineered binding domain
GVSDFYKRLINRAKTVEGVNTLKADI

SEQ ID NO: 110 - protein, 26 aa, Artificial
  Engineered binding domain
GVSDFYKNLINRAKTVEGVNTLKADI

SEQ ID NO: 111 - protein, 26 aa, Artificial
  Engineered binding domain
GVSDYYKNLINRAKTVEGVNSLKSHI

SEQ ID NO: 112 - protein, 26 aa, Artificial
  Engineered binding domain
GVSDFYKRLINRAKTVGGVQSLKSEI

SEQ ID NO: 113 - protein, 26 aa, Artificial
  Engineered binding domain
GVSDFYKNVINRAKTVEGVDSLKAEI

SEQ ID NO: 114 - protein, 26 aa, Artificial
  Engineered binding domain
GVSDFYKNLINRARTVEGVQTLKSDI

SEQ ID NO: 115 - protein, 26 aa, Artificial
  Engineered binding domain
GVSDFYKKFINKAKTVEGVETLKSEI

SEQ ID NO: 116 - protein, 26 aa, Artificial
  Engineered binding domain
GVSDFYKSLINRAKTVEGVHSLKDEI

SEQ ID NO: 117 - protein, 26 aa, Artificial
  Engineered binding domain
GVSDYYKNVINKAKTVEGVSSLKAEI

SEQ ID NO: 118 - protein, 26 aa, Artificial
  Engineered binding domain
GVSDFYKSLINRAKTVEGVDALKSHI

SEQ ID NO: 119 - protein, 26 aa, Artificial
  Engineered binding domain
GVSDFYKNLINKAKTVEGVSTLKHDI

SEQ ID NO: 120 - protein, 26 aa, Artificial
  Engineered binding domain
GVSDFYKNLINRAKTVEGVSTLKHDI

SEQ ID NO: 121 - protein, 26 aa, Artificial
  Engineered binding domain
GVSDFYKNLINRAKTVEGVQALKSEI

SEQ ID NO: 122 - protein, 26 aa, Artificial
  Engineered binding domain
GVSDYYKSLINKAKTVEGVDSLKVHI

SEQ ID NO: 123 - protein, 26 aa, Artificial
  Engineered binding domain
GVSDFYKNLINRAKTVEGVQSLKTEI

SEQ ID NO: 124 - protein, 26 aa, Artificial
  Engineered binding domain
GVSDYYKNLINRAKTVEGVDALKTHI

SEQ ID NO: 125 - protein, 26 aa, Artificial
  Engineered binding domain
GVSDFYKSMINRAKTVEGVDSLKTHI

SEQ ID NO: 126 - protein, 26 aa, Artificial
  Engineered binding domain
GVSDFYKNLINRAKTVEGVTTLKTDI

SEQ ID NO: 127 - protein, 26 aa, Artificial
  Engineered binding domain
GVSDFYKNLINRAKTVEGVESLKDHI

SEQ ID NO: 128 - protein, 26 aa, Artificial
  Engineered binding domain
GVSDFYKSYINRAKTVEGVHTLKGHI

SEQ ID NO: 129 - protein, 26 aa, Artificial
  Engineered binding domain
GVSDFYKNLINRAKTVEGVQTLKSDI

SEQ ID NO: 130 - protein, 26 aa, Artificial
  Engineered binding domain
GVSDFYKNLINRAKTVEGVNSLKSHI

SEQ ID NO: 131 - protein, 26 aa, Artificial
  Engineered binding domain
GVSDFYKNLINRAKTVEGVNTLKHDI

SEQ ID NO: 132 - protein, 26 aa, Artificial
  Engineered binding domain
GVSDFYKNLINRAKTVEGVESLKGEI

SEQ ID NO: 133 - protein, 26 aa, Artificial
  Engineered binding domain
GVSDFYKNLINKAKTVEGVHTLKHDI

SEQ ID NO: 134 - protein, 26 aa, Artificial
  Engineered binding domain
GVSDFYKNLINKAKTVEGVDALKVHI

SEQ ID NO: 135 - protein, 26 aa, Artificial
  Engineered binding domain
GVSDYYKRLINRAKTVEGVHALKAEI

SEQ ID NO: 136 - protein, 26 aa, Artificial
  Engineered binding domain
GVSDYYKNLINRARTVEGVDTLKHDI

SEQ ID NO: 137 - protein, 26 aa, Artificial
  Engineered binding domain
GVSDFYKKVINRARTVEGVQALKADI

SEQ ID NO: 138 - protein, 26 aa, Artificial
  Engineered binding domain
GVSDFYKNLINRAKTVEGVESLKADI

SEQ ID NO: 139 - protein, 26 aa, Artificial
  Engineered binding domain
GVSDYYKNLINKAKTVEGVDALKAHI

SEQ ID NO: 140 - protein, 26 aa, Artificial
  Engineered binding domain
GVSDYYKNLINRAKTVEGVESLKTHI

SEQ ID NO: 141 - protein, 26 aa, Artificial
  Engineered binding domain
GVSDFYKNLINRARTVEGVDSLKVEI

SEQ ID NO: 142 - protein, 26 aa, Artificial
  Engineered binding domain
GVSDFYKNVINRAKTVEGVSALKREI

SEQ ID NO: 143 - protein, 26 aa, Artificial
  Engineered binding domain
GVSDFYKNLINRAKTVEGVNALKSDI

SEQ ID NO: 144 - protein, 26 aa, Artificial
  Engineered binding domain
GVSDFYKNLINRAKTVEGVSALKQEI

SEQ ID NO: 145 - protein, 26 aa, Artificial
  Engineered binding domain
GVSDFYKNLINRAKTVEGVQSLKDHI

SEQ ID NO: 146 - protein, 26 aa, Artificial
  Engineered binding domain
GVSDFYKNLINRAKTVEGVDALKCHI

SEQ ID NO: 147 - protein, 26 aa, Artificial
  Engineered binding domain
GVSDYYKRLINKAKTVEGVNALKTHI

SEQ ID NO: 148 - protein, 26 aa, Artificial
  Engineered binding domain
GVSDFYKNVINKAKTVEGVEALKADI

SEQ ID NO: 149 - protein, 26 aa, Artificial
  Engineered binding domain
GVSDFYKNLINRAKTVEGVETLKRDI

SEQ ID NO: 150 - protein, 26 aa, Artificial
  Engineered binding domain
GVSDFYKNLINRARTVEGVQTLKTDI

SEQ ID NO: 151 - protein, 26 aa, Artificial
  Engineered binding domain
GVSDFYKRLINKAKTVEGVNALKHHI

SEQ ID NO: 152 - protein, 26 aa, Artificial
  Engineered binding domain
GVSDYYKNLINRAKTVEGVQALKAHI

SEQ ID NO: 153 - protein, 26 aa, Artificial
  Engineered binding domain
GVSDFYKNVINRAKTVEGVNSLKNHI

SEQ ID NO: 154 - protein, 26 aa, Artificial
  Engineered binding domain
GVSDFYKSLINRARTVEGVDSLKRHI

SEQ ID NO: 155 - protein, 26 aa, Artificial
  Engineered binding domain
GVSDYYKNLINKAKTVEGVEALKLHI

SEQ ID NO: 156 - protein, 26 aa, Artificial
  Engineered binding domain
GVSDFYKNLINRAKTVEGVDALKAHI

SEQ ID NO: 157 - protein, 26 aa, Artificial
  Engineered binding domain
GVSDYYKNLINKAKTVEGVQALKAHI

SEQ ID NO: 158 - protein, 26 aa, Artificial
  Engineered binding domain
GVSDFYKRLINRAKTVEGVHALKGHI

SEQ ID NO: 159 - protein, 26 aa, Artificial
  Engineered binding domain
GVSDFYKRVINRAKTVEGVHALKDHI

SEQ ID NO: 160 - protein, 26 aa, Artificial
  Engineered binding domain
GVSDYYKRIINRAKTVEGVRALKLHI

SEQ ID NO: 161 - protein, 26 aa, Artificial
  Engineered binding domain
GVSDFYKRVINRAKTVEGVSALKHEI

SEQ ID NO: 162 - protein, 26 aa, Artificial
  Engineered binding domain
GVSDYYKRLINRAKTVEGVNSLKSHI

SEQ ID NO: 163 - protein, 26 aa, Artificial
  Engineered binding domain
GVSDFYKRLINRAKTVGGVQSLKSEI

SEQ ID NO: 164 - protein, 26 aa, Artificial
  Engineered binding domain
GVSDFYKRLINRAKTVEGVSSLKGHI

SEQ ID NO: 165 - protein, 26 aa, Artificial
  Engineered binding domain
GVSDFYKRVINRAKTVEGVDSLKAEI

SEQ ID NO: 166 - protein, 26 aa, Artificial
  Engineered binding domain
GVSDFYKRLINRARTVEGVQTLKSDI

SEQ ID NO: 167 - protein, 26 aa, Artificial
  Engineered binding domain
GVSDFYKRFINKAKTVEGVETLKSEI

SEQ ID NO: 168 - protein, 26 aa, Artificial
  Engineered binding domain
GVSDFYKRLINRAKTVEGVHSLKDEI

SEQ ID NO: 169 - protein, 26 aa, Artificial
  Engineered binding domain
GVSDYYKRVINKAKTVEGVSSLKAEI

SEQ ID NO: 170 - protein, 26 aa, Artificial
  Engineered binding domain
GVSDFYKRLINRAKTVEGVDALKSHI

SEQ ID NO: 171 - protein, 26 aa, Artificial
  Engineered binding domain
GVSDFYKRLINKAKTVEGVSTLKHDI

SEQ ID NO: 172 - protein, 26 aa, Artificial
  Engineered binding domain
GVSDFYKRLINRAKTVEGVSTLKHDI

SEQ ID NO: 173 - protein, 26 aa, Artificial
  Engineered binding domain
GVSDFYKRLINRAKTVEGVQALKSEI

SEQ ID NO: 174 - protein, 26 aa, Artificial
  Engineered binding domain
GVSDYYKRLINKAKTVEGVDSLKVHI

SEQ ID NO: 175 - protein, 26 aa, Artificial
  Engineered binding domain
GVSDFYKRLINRAKTVEGVQSLKTEI

SEQ ID NO: 176 - protein, 26 aa, Artificial
  Engineered binding domain
GVSDYYKRLINRAKTVEGVDALKTHI

SEQ ID NO: 177 - protein, 26 aa, Artificial
  Engineered binding domain
GVSDFYKRMINRAKTVEGVDSLKTHI

SEQ ID NO: 178 - protein, 26 aa, Artificial
  Engineered binding domain
GVSDFYKRLINRAKTVEGVTTLKTDI

SEQ ID NO: 179 - protein, 26 aa, Artificial
  Engineered binding domain
GVSDFYKRLINRAKTVEGVESLKDHI

SEQ ID NO: 180 - protein, 26 aa, Artificial
  Engineered binding domain
GVSDFYKRYINRAKTVEGVHTLKGHI

SEQ ID NO: 181 - protein, 26 aa, Artificial
  Engineered binding domain
GVSDFYKRLINRAKTVEGVQTLKSDI

SEQ ID NO: 182 - protein, 26 aa, Artificial
  Engineered binding domain
GVSDFYKRLINRAKTVEGVNSLKSHI

SEQ ID NO: 183 - protein, 26 aa, Artificial
  Engineered binding domain
GVSDFYKRLINRAKTVEGVNTLKHDI

SEQ ID NO: 184 - protein, 26 aa, Artificial
  Engineered binding domain
GVSDFYKRLINRAKTVEGVESLKGEI

SEQ ID NO: 185 - protein, 26 aa, Artificial
  Engineered binding domain
GVSDFYKRLINKAKTVEGVHTLKHDI

SEQ ID NO: 186 - protein, 26 aa, Artificial
  Engineered binding domain
GVSDYYKRLINKAKTVEGVSALKMHI

SEQ ID NO: 187 - protein, 26 aa, Artificial
  Engineered binding domain
GVSDFYKRLINKAKTVEGVDALKVHI

SEQ ID NO: 188 - protein, 26 aa, Artificial
  Engineered binding domain
GVSDYYKRLINRAKTVEGVHALKAEI

SEQ ID NO: 189 - protein, 26 aa, Artificial
  Engineered binding domain
GVSDYYKRLINRARTVEGVDTLKHDI

SEQ ID NO: 190 - protein, 26 aa, Artificial
  Engineered binding domain
GVSDFYKRVINRARTVEGVQALKADI

SEQ ID NO: 191 - protein, 26 aa, Artificial
  Engineered binding domain
GVSDFYKRLINRAKTVEGVESLKADI

SEQ ID NO: 192 - protein, 26 aa, Artificial
  Engineered binding domain
GVSDYYKRLINKAKTVEGVDALKAHI

SEQ ID NO: 193 - protein, 26 aa, Artificial
  Engineered binding domain
GVSDYYKRLINRAKTVEGVESLKTHI

SEQ ID NO: 194 - protein, 26 aa, Artificial
  Engineered binding domain
GVSDFYKRLINRARTVEGVDSLKVEI

SEQ ID NO: 195 - protein, 26 aa, Artificial
  Engineered binding domain
GVSDFYKRVINRAKTVEGVSALKREI

SEQ ID NO: 196 - protein, 26 aa, Artificial
  Engineered binding domain
GVSDFYKRLINRAKTVEGVNALKSDI

SEQ ID NO: 197 - protein, 26 aa, Artificial
  Engineered binding domain
GVSDFYKRLINRAKTVEGVSALKQEI

SEQ ID NO: 198 - protein, 26 aa, Artificial
  Engineered binding domain
GVSDFYKRLINRAKTVEGVQSLKDHI

SEQ ID NO: 199 - protein, 26 aa, Artificial
  Engineered binding domain
GVSDFYKRLINRAKTVEGVDALKCHI

SEQ ID NO: 200 - protein, 26 aa, Artificial
  Engineered binding domain
GVSDYYKRLINKAKTVEGVNALKTHI

SEQ ID NO: 201 - protein, 26 aa, Artificial
  Engineered binding domain
GVSDFYKRVINKAKTVEGVEALKADI

SEQ ID NO: 202 - protein, 26 aa, Artificial
  Engineered binding domain
GVSDFYKRLINRAKTVEGVETLKRDI

SEQ ID NO: 203 - protein, 26 aa, Artificial
  Engineered binding domain
GVSDFYKRLINRARTVEGVQTLKTDI

SEQ ID NO: 204 - protein, 26 aa, Artificial
  Engineered binding domain
GVSDFYKRLINKAKTVEGVNALKHHI

SEQ ID NO: 205 - protein, 26 aa, Artificial
  Engineered binding domain
GVSDYYKRLINRAKTVEGVQALKAHI

SEQ ID NO: 206 - protein, 26 aa, Artificial
  Engineered binding domain
GVSDFYKRVINRAKTVEGVNSLKNHI

SEQ ID NO: 207 - protein, 26 aa, Artificial
  Engineered binding domain
GVSDFYKRLINRARTVEGVDSLKRHI

SEQ ID NO: 208 - protein, 26 aa, Artificial
  Engineered binding domain
GVSDYYKRLINKAKTVEGVEALKLHI

SEQ ID NO: 209 - protein, 26 aa, Artificial
  Engineered binding domain
GVSDFYKRLINRAKTVEGVDALKAHI

SEQ ID NO: 210 - protein, 26 aa, Artificial
  Engineered binding domain
GVSDYYKRLINKAKTVEGVQALKAHI

SEQ ID NO: 211 - protein, 26 aa, Artificial
  Engineered binding domain
GVSDFYKRVINRAKTVEGVSALKHHI

SEQ ID NO: 212 - protein, 26 aa, Artificial
  Engineered binding domain
GVSDFYKRLINRAKTVEGVNTLKAHI

SEQ ID NO: 213 - protein, 26 aa, Artificial
  Engineered binding domain
GVSDFYKRLINRAKTVGGVQSLKSHI

SEQ ID NO: 214 - protein, 26 aa, Artificial
  Engineered binding domain
GVSDFYKRVINRAKTVEGVDSLKAHI

SEQ ID NO: 215 - protein, 26 aa, Artificial
  Engineered binding domain
GVSDFYKRLINRARTVEGVQTLKSHI

SEQ ID NO: 216 - protein, 26 aa, Artificial
  Engineered binding domain
GVSDFYKRFINKAKTVEGVETLKSHI

SEQ ID NO: 217 - protein, 26 aa, Artificial
  Engineered binding domain
GVSDFYKRLINRAKTVEGVHSLKDHI

SEQ ID NO: 218 - protein, 26 aa, Artificial
  Engineered binding domain
GVSDYYKRVINKAKTVEGVSSLKAHI

SEQ ID NO: 219 - protein, 26 aa, Artificial
  Engineered binding domain
GVSDFYKRLINKAKTVEGVSTLKHHI

SEQ ID NO: 220 - protein, 26 aa, Artificial
  Engineered binding domain
GVSDFYKRLINRAKTVEGVSTLKHHI

SEQ ID NO: 221 - protein, 26 aa, Artificial
  Engineered binding domain
GVSDFYKRLINRAKTVEGVQALKSHI

SEQ ID NO: 222 - protein, 26 aa, Artificial
  Engineered binding domain
GVSDFYKRLINRAKTVEGVQSLKTHI

SEQ ID NO: 223 - protein, 26 aa, Artificial
  Engineered binding domain
GVSDFYKRLINRAKTVEGVTTLKTHI

SEQ ID NO: 224 - protein, 26 aa, Artificial
  Engineered binding domain
GVSDFYKRLINRAKTVEGVQTLKSHI

SEQ ID NO: 225 - protein, 26 aa, Artificial
  Engineered binding domain
GVSDFYKRLINRAKTVEGVNTLKHHI

SEQ ID NO: 226 - protein, 26 aa, Artificial
  Engineered binding domain
GVSDFYKRLINRAKTVEGVESLKGHI

SEQ ID NO: 227 - protein, 26 aa, Artificial
  Engineered binding domain
GVSDFYKRLINKAKTVEGVHTLKHHI

SEQ ID NO: 228 - protein, 26 aa, Artificial
  Engineered binding domain
GVSDYYKRLINRAKTVEGVHALKAHI

SEQ ID NO: 229 - protein, 26 aa, Artificial
  Engineered binding domain
GVSDYYKRLINRARTVEGVDTLKHHI

SEQ ID NO: 230 - protein, 26 aa, Artificial
  Engineered binding domain
GVSDFYKRVINRARTVEGVQALKAHI

SEQ ID NO: 231 - protein, 26 aa, Artificial
  Engineered binding domain
GVSDFYKRLINRAKTVEGVESLKAHI

SEQ ID NO: 232 - protein, 26 aa, Artificial
  Engineered binding domain
GVSDFYKRLINRARTVEGVDSLKVHI

SEQ ID NO: 233 - protein, 26 aa, Artificial
  Engineered binding domain
GVSDFYKRVINRAKTVEGVSALKRHI

SEQ ID NO: 234 - protein, 26 aa, Artificial
  Engineered binding domain
GVSDFYKRLINRAKTVEGVNALKSHI

SEQ ID NO: 235 - protein, 26 aa, Artificial
  Engineered binding domain
GVSDFYKRLINRAKTVEGVSALKQHI

SEQ ID NO: 236 - protein, 26 aa, Artificial
  Engineered binding domain
GVSDFYKRVINKAKTVEGVEALKAHI

SEQ ID NO: 237 - protein, 26 aa, Artificial
  Engineered binding domain
GVSDFYKRLINRAKTVEGVETLKRHI

SEQ ID NO: 238 - protein, 26 aa, Artificial
  Engineered binding domain
GVSDFYKRLINRARTVEGVQTLKTHI

SEQ ID NO: 239 - protein, 26 aa, Artificial
  Engineered binding domain
GVSDFYKRLINKAKTVEGVEALKLHI

SEQ ID NO: 240 - protein, 26 aa, Artificial
  Engineered binding domain
GVSDYYKNLINRARTVEGVEALKLHI

SEQ ID NO: 241 - protein, 26 aa, Artificial
  Engineered binding domain
GVSDYYKNIINRAKTVEGVEALKLHI

SEQ ID NO: 242 - protein, 26 aa, Artificial
  Engineered binding domain
GVSDFYKNLINRAKTVEGVEALKLHI

SEQ ID NO: 243 - protein, 26 aa, Artificial
  Engineered binding domain
GVSDFYKNVINKAKTVEGVEALKLHI

SEQ ID NO: 244 - protein, 26 aa, Artificial
  Engineered binding domain
GVSDYYKNLINRAKTVEGVEALKLHI

SEQ ID NO: 245 - protein, 26 aa, Artificial
  Engineered binding domain
GVSDYYKNLINRARTVEGVHALIDHI

SEQ ID NO: 246 - protein, 26 aa, Artificial
  Engineered binding domain
GVSDFYKRLINKAKTVEGVEALKLHI

SEQ ID NO: 247 - protein, 26 aa, Artificial
  Engineered binding domain
GVSDYYKRLISKAKTVEGVKALISEI

SEQ ID NO: 248 - protein, 26 aa, Artificial
  Engineered binding domain
GVSDFYKRLINKAKTVEGVEALKLHI

SEQ ID NO: 249 - protein, 26 aa, Artificial
  Engineered binding domain
GVSDYYKRLISKAKTVEGVKALISEI

SEQ ID NO: 250 - protein, 26 aa, Artificial
  Engineered binding domain
GVSDYYKRLISKAKTVEGVKALISEI

SEQ ID NO: 251 - protein, 26 aa, Artificial
  Engineered binding domain
GVSDYYKRLISKAKTVEGVKALISEI

SEQ ID NO: 252 - protein, 26 aa, Artificial
  Engineered binding domain
GVSDYYKRLISKAKTVEGVKALISEI

SEQ ID NO: 253 - protein, 26 aa, Artificial
  Engineered binding domain
GVSDYYKRLISKAKTVEGVKALISEI

SEQ ID NO: 254 - protein, 26 aa, Artificial
  Engineered binding domain
GVSDYYKRLISKAKTVEGVKALISEI

SEQ ID NO: 255 - protein, 26 aa, Artificial
  Engineered binding domain
GVSDYYKRLISKAKTVEGVKALISEI

SEQ ID NO: 256 - protein, 26 aa, Artificial
  Engineered binding domain
GVSDYYKRLISKAKTVEGVKALISEI

SEQ ID NO: 257 - protein, 26 aa, Artificial
  Engineered binding domain
GVSDYYKRLISKAKTVEGVKALISEI

SEQ ID NO: 258 - protein, 46 aa, Artificial
  Engineered binding domain
LAEAKVLANRELDKYGVSDFYKNLINRAKTVEGVHALIGHILAALP

SEQ ID NO: 259 - protein, 46 aa, Artificial
  Engineered binding domain
LAEAKVLANRELDKYGVSDFYKNVINRAKTVEGVHALIDHILAALP

SEQ ID NO: 260 - protein, 46 aa, Artificial
  Engineered binding domain
LAEAKVLANRELDKYGVSDYYKNIINRAKTVEGVRALKLHILAALP

SEQ ID NO: 261 - protein, 46 aa, Artificial
  Engineered binding domain
LAEAKVLANRELDKYGVSDFYKNVINRAKTVEGVSALIHEILAALP

SEQ ID NO: 262 - protein, 46 aa, Artificial
  Engineered binding domain
LAEAKVLANRELDKYGVSDFYKRLINRAKTVEGVNTLIADILAALP

SEQ ID NO: 263 - protein, 46 aa, Artificial
  Engineered binding domain
LAEAKVLANRELDKYGVSDFYKNLINRAKTVEGVNTLIADILAALP

SEQ ID NO: 264 - protein, 46 aa, Artificial
  Engineered binding domain
LAEAKVLANRELDKYGVSDYYKNLINRAKTVEGVNSLISHILAALP

SEQ ID NO: 265 - protein, 46 aa, Artificial
  Engineered binding domain
LAEAKVLANRELDKYGVSDFYKRLINRAKTVGGVQSLISEILAALP

SEQ ID NO: 266 - protein, 46 aa, Artificial
  Engineered binding domain
LAEAKVLANRELDKYGVSDFYKNLINRAKTVEGVSSLKGHILAALP

SEQ ID NO: 267 - protein, 46 aa, Artificial
  Engineered binding domain
LAEAKVLANRELDKYGVSDFYKNVINRAKTVEGVDSLIAEILAALP

SEQ ID NO: 268 - protein, 46 aa, Artificial
  Engineered binding domain
LAEAKVLANRELDKYGVSDFYKNLINRARTVEGVQTLISDILAALP

SEQ ID NO: 269 - protein, 46 aa, Artificial
  Engineered binding domain
LAEAKVLANRELDKYGVSDFYKKFINKAKTVEGVETLISEILAALP

SEQ ID NO: 270 - protein, 46 aa, Artificial
  Engineered binding domain
LAEAKVLANRELDKYGVSDFYKSLINRAKTVEGVHSLTDEILAALP

SEQ ID NO: 271 - protein, 46 aa, Artificial
  Engineered binding domain
LAEAKVLANRELDKYGVSDYYKNVINKAKTVEGVSSLTAEILAALP

SEQ ID NO: 272 - protein, 46 aa, Artificial
  Engineered binding domain
LAEAKVLANRELDKYGVSDFYKSLINRAKTVEGVDALTSHILAALP

SEQ ID NO: 273 - protein, 46 aa, Artificial
  Engineered binding domain
LAEAKVLANRELDKYGVSDFYKNLINKAKTVEGVSTLIHDILAALP

SEQ ID NO: 274 - protein, 46 aa, Artificial
  Engineered binding domain
LAEAKVLANRELDKYGVSDFYKNLINRAKTVEGVSTLIHDILAALP

SEQ ID NO: 275 - protein, 46 aa, Artificial
  Engineered binding domain
LAEAKVLANRELDKYGVSDFYKNLINRAKTVEGVQALISEILAALP

SEQ ID NO: 276 - protein, 46 aa, Artificial
  Engineered binding domain
LAEAKVLANRELDKYGVSDYYKSLINKAKTVEGVDSLIVHILAALP

SEQ ID NO: 277 - protein, 46 aa, Artificial
  Engineered binding domain
LAEAKVLANRELDKYGVSDFYKNLINRAKTVEGVQSLITEILAALP

SEQ ID NO: 278 - protein, 46 aa, Artificial
  Engineered binding domain
LAEAKVLANRELDKYGVSDYYKNLINRAKTVEGVDALITHILAALP

SEQ ID NO: 279 - protein, 46 aa, Artificial
  Engineered binding domain
LAEAKVLANRELDKYGVSDFYKSMINRAKTVEGVDSLITHILAALP

SEQ ID NO: 280 - protein, 46 aa, Artificial
  Engineered binding domain
LAEAKVLANRELDKYGVSDFYKNLINRAKTVEGVTTLTTDILAALP

SEQ ID NO: 281 - protein, 46 aa, Artificial
  Engineered binding domain
LAEAKVLANRELDKYGVSDFYKNLINRAKTVEGVESLIDHILAALP

SEQ ID NO: 282 - protein, 46 aa, Artificial
  Engineered binding domain
LAEAKVLANRELDKYGVSDFYKSYINRAKTVEGVHTLIGHILAALP

SEQ ID NO: 283 - protein, 46 aa, Artificial
  Engineered binding domain
LAEAKVLANRELDKYGVSDFYKNLINRAKTVEGVQTLISDILAALP

SEQ ID NO: 284 - protein, 46 aa, Artificial
  Engineered binding domain
LAEAKVLANRELDKYGVSDFYKNLINRAKTVEGVNSLTSHILAALP

SEQ ID NO: 285 - protein, 46 aa, Artificial
  Engineered binding domain
LAEAKVLANRELDKYGVSDFYKNLINRAKTVEGVNTLIHDILAALP

SEQ ID NO: 286 - protein, 46 aa, Artificial
  Engineered binding domain
LAEAKVLANRELDKYGVSDFYKNLINRAKTVEGVESLIGEILAALP

SEQ ID NO: 287 - protein, 46 aa, Artificial
  Engineered binding domain
LAEAKVLANRELDKYGVSDFYKNLINKAKTVEGVHTLIHDILAALP

SEQ ID NO: 288 - protein, 46 aa, Artificial
  Engineered binding domain
LAEAKVLANRELDKYGVSDYYKNLINKAKTVEGVSALKMHILAALP

SEQ ID NO: 289 - protein, 46 aa, Artificial
  Engineered binding domain
LAEAKVLANRELDKYGVSDFYKNLINKAKTVEGVDALIVHILAALP

SEQ ID NO: 290 - protein, 46 aa, Artificial
  Engineered binding domain
LAEAKVLANRELDKYGVSDYYKRLINRAKTVEGVHALIAEILAALP

SEQ ID NO: 291 - protein, 46 aa, Artificial
  Engineered binding domain
LAEAKVLANRELDKYGVSDYYKNLINRARTVEGVDTLIHDILAALP

SEQ ID NO: 292 - protein, 46 aa, Artificial
  Engineered binding domain
LAEAKVLANRELDKYGVSDFYKKVINRARTVEGVQALIADILAALP

SEQ ID NO: 293 - protein, 46 aa, Artificial
  Engineered binding domain
LAEAKVLANRELDKYGVSDFYKNLINRAKTVEGVESLIADILAALP

SEQ ID NO: 294 - protein, 46 aa, Artificial
  Engineered binding domain
LAEAKVLANRELDKYGVSDYYKNLINKAKTVEGVDALIAHILAALP

SEQ ID NO: 295 - protein, 46 aa, Artificial
  Engineered binding domain
LAEAKVLANRELDKYGVSDYYKNLINRAKTVEGVESLITHILAALP

SEQ ID NO: 296 - protein, 46 aa, Artificial
  Engineered binding domain
LAEAKVLANRELDKYGVSDFYKNLINRARTVEGVDSLIVEILAALP

SEQ ID NO: 297 - protein, 46 aa, Artificial
  Engineered binding domain
LAEAKVLANRELDKYGVSDFYKNVINRAKTVEGVSALIREILAALP

SEQ ID NO: 298 - protein, 46 aa, Artificial
  Engineered binding domain
LAEAKVLANRELDKYGVSDFYKNLINRAKTVEGVNALISDILAALP

SEQ ID NO: 299 - protein, 46 aa, Artificial
  Engineered binding domain
LAEAKVLANRELDKYGVSDFYKNLINRAKTVEGVSALIQEILAALP

SEQ ID NO: 300 - protein, 46 aa, Artificial
  Engineered binding domain
LAEAKVLANRELDKYGVSDFYKNLINRAKTVEGVQSLIDHILAALP

SEQ ID NO: 301 - protein, 46 aa, Artificial
  Engineered binding domain
LAEAKVLANRELDKYGVSDFYKNLINRAKTVEGVDALICHILAALP

SEQ ID NO: 302 - protein, 46 aa, Artificial
  Engineered binding domain
LAEAKVLANRELDKYGVSDYYKRLINKAKTVEGVNALITHILAALP

SEQ ID NO: 303 - protein, 46 aa, Artificial
  Engineered binding domain
LAEAKVLANRELDKYGVSDFYKNVINKAKTVEGVEALIADILAALP

SEQ ID NO: 304 - protein, 46 aa, Artificial
  Engineered binding domain
LAEAKVLANRELDKYGVSDFYKNLINRAKTVEGVETLIRDILAALP

SEQ ID NO: 305 - protein, 46 aa, Artificial
  Engineered binding domain
LAEAKVLANRELDKYGVSDFYKNLINRARTVEGVQTLITDILAALP

SEQ ID NO: 306 - protein, 46 aa, Artificial
  Engineered binding domain
LAEAKVLANRELDKYGVSDFYKRLINKAKTVEGVNALTHHILAALP

SEQ ID NO: 307 - protein, 46 aa, Artificial
  Engineered binding domain
LAEAKVLANRELDKYGVSDYYKNLINRAKTVEGVQALIAHILAALP

SEQ ID NO: 308 - protein, 46 aa, Artificial
  Engineered binding domain
LAEAKVLANRELDKYGVSDFYKNVINRAKTVEGVNSLINHILAALP

SEQ ID NO: 309 - protein, 46 aa, Artificial
  Engineered binding domain
LAEAKVLANRELDKYGVSDFYKSLINRARTVEGVDSLIRHILAALP

SEQ ID NO: 310 - protein, 46 aa, Artificial
  Engineered binding domain
LAEAKVLANRELDKYGVSDYYKNLINKAKTVEGVEALTLHILAALP

SEQ ID NO: 311 - protein, 46 aa, Artificial
  Engineered binding domain
LAEAKVLANRELDKYGVSDFYKNLINRAKTVEGVDALIAHILAALP

SEQ ID NO: 312 - protein, 46 aa, Artificial
  Engineered binding domain
LAEAKVLANRELDKYGVSDYYKNLINKAKTVEGVQALIAHILAALP

SEQ ID NO: 313 - protein, 46 aa, Artificial
  Engineered binding domain
LAEAKVLANRELDKYGVSDFYKRLINRAKTVEGVHALIGHILAALP

SEQ ID NO: 314 - protein, 46 aa, Artificial
  Engineered binding domain
LAEAKVLANRELDKYGVSDFYKRVINRAKTVEGVHALIDHILAALP

SEQ ID NO: 315 - protein, 46 aa, Artificial
  Engineered binding domain
LAEAKVLANRELDKYGVSDYYKRIINRAKTVEGVRALKLHILAALP

SEQ ID NO: 316 - protein, 46 aa, Artificial
  Engineered binding domain
LAEAKVLANRELDKYGVSDFYKRVINRAKTVEGVSALIHEILAALP

SEQ ID NO: 317 - protein, 46 aa, Artificial; identical to SEQ ID NO: 262
  Engineered binding domain

SEQ ID NO: 318 - protein, 46 aa, Artificial
  Engineered binding domain
LAEAKVLANRELDKYGVSDYYKRLINRAKTVEGVNSLISHILAALP

SEQ ID NO: 319 - protein, 46 aa, Artificial
  Engineered binding domain
LAEAKVLANRELDKYGVSDFYKRLINRAKTVEGVSSLKGHILAALP

SEQ ID NO: 320 - protein, 46 aa, Artificial
  Engineered binding domain
LAEAKVLANRELDKYGVSDFYKRVINRAKTVEGVDSLIAEILAALP

SEQ ID NO: 321 - protein, 46 aa, Artificial
  Engineered binding domain
LAEAKVLANRELDKYGVSDFYKRLINRARTVEGVQTLISDILAALP

SEQ ID NO: 322 - protein, 46 aa, Artificial
  Engineered binding domain
LAEAKVLANRELDKYGVSDFYKRFINKAKTVEGVETLISEILAALP

SEQ ID NO: 323 - protein, 46 aa, Artificial
  Engineered binding domain
LAEAKVLANRELDKYGVSDFYKRLINRAKTVEGVHSLTDEILAALP

SEQ ID NO: 324 - protein, 46 aa, Artificial
  Engineered binding domain
LAEAKVLANRELDKYGVSDYYKRVINKAKTVEGVSSLTAEILAALP

SEQ ID NO: 325 - protein, 46 aa, Artificial
  Engineered binding domain
LAEAKVLANRELDKYGVSDFYKRLINRAKTVEGVDALTSHILAALP

SEQ ID NO: 326 - protein, 46 aa, Artificial
  Engineered binding domain
LAEAKVLANRELDKYGVSDFYKRLINKAKTVEGVSTLIHDILAALP

SEQ ID NO: 327 - protein, 46 aa, Artificial
  Engineered binding domain
LAEAKVLANRELDKYGVSDFYKRLINRAKTVEGVSTLIHDILAALP

SEQ ID NO: 328 - protein, 46 aa, Artificial
  Engineered binding domain
LAEAKVLANRELDKYGVSDFYKRLINRAKTVEGVQALISEILAALP

SEQ ID NO: 329 - protein, 46 aa, Artificial
  Engineered binding domain
LAEAKVLANRELDKYGVSDYYKRLINKAKTVEGVDSLIVHILAALP

SEQ ID NO: 330 - protein, 46 aa, Artificial
  Engineered binding domain
LAEAKVLANRELDKYGVSDFYKRLINRAKTVEGVQSLITEILAALP

SEQ ID NO: 331 - protein, 46 aa, Artificial
  Engineered binding domain
LAEAKVLANRELDKYGVSDYYKRLINRAKTVEGVDALITHILAALP

SEQ ID NO: 332 - protein, 46 aa, Artificial
  Engineered binding domain
LAEAKVLANRELDKYGVSDFYKRMINRAKTVEGVDSLITHILAALP

SEQ ID NO: 333 - protein, 46 aa, Artificial
  Engineered binding domain
LAEAKVLANRELDKYGVSDFYKRLINRAKTVEGVTTLTTDILAALP

SEQ ID NO: 334 - protein, 46 aa, Artificial
  Engineered binding domain
LAEAKVLANRELDKYGVSDFYKRLINRAKTVEGVESLIDHILAALP

SEQ ID NO: 335 - protein, 46 aa, Artificial
  Engineered binding domain
LAEAKVLANRELDKYGVSDFYKRYINRAKTVEGVHTLIGHILAALP

SEQ ID NO: 336 - protein, 46 aa, Artificial
  Engineered binding domain
LAEAKVLANRELDKYGVSDFYKRLINRAKTVEGVQTLISDILAALP

SEQ ID NO: 337 - protein, 46 aa, Artificial
  Engineered binding domain
LAEAKVLANRELDKYGVSDFYKRLINRAKTVEGVNSLTSHILAALP

SEQ ID NO: 338 - protein, 46 aa, Artificial
  Engineered binding domain
LAEAKVLANRELDKYGVSDFYKRLINRAKTVEGVNTLIHDILAALP

SEQ ID NO: 339 - protein, 46 aa, Artificial
  Engineered binding domain
LAEAKVLANRELDKYGVSDFYKRLINRAKTVEGVESLIGEILAALP

SEQ ID NO: 340 - protein, 46 aa, Artificial
  Engineered binding domain
LAEAKVLANRELDKYGVSDFYKRLINKAKTVEGVHTLIHDILAALP

SEQ ID NO: 341 - protein, 46 aa, Artificial
  Engineered binding domain
LAEAKVLANRELDKYGVSDYYKRLINKAKTVEGVSALKMHILAALP

SEQ ID NO: 342 - protein, 46 aa, Artificial
  Engineered binding domain
LAEAKVLANRELDKYGVSDFYKRLINKAKTVEGVDALIVHILAALP

SEQ ID NO: 343 - protein, 46 aa, Artificial
  Engineered binding domain
LAEAKVLANRELDKYGVSDYYKRLINRARTVEGVDTLIHDILAALP

SEQ ID NO: 344 - protein, 46 aa, Artificial
  Engineered binding domain
LAEAKVLANRELDKYGVSDFYKRVINRARTVEGVQALIADILAALP

SEQ ID NO: 345 - protein, 46 aa, Artificial
  Engineered binding domain
LAEAKVLANRELDKYGVSDFYKRLINRAKTVEGVESLIADILAALP

SEQ ID NO: 346 - protein, 46 aa, Artificial
  Engineered binding domain
LAEAKVLANRELDKYGVSDYYKRLINKAKTVEGVDALIAHILAALP

SEQ ID NO: 347 - protein, 46 aa, Artificial
  Engineered binding domain
LAEAKVLANRELDKYGVSDYYKRLINRAKTVEGVESLITHILAALP

SEQ ID NO: 348 - protein, 46 aa, Artificial
  Engineered binding domain
LAEAKVLANRELDKYGVSDFYKRLINRARTVEGVDSLIVEILAALP

SEQ ID NO: 349 - protein, 46 aa, Artificial
  Engineered binding domain
LAEAKVLANRELDKYGVSDFYKRVINRAKTVEGVSALIREILAALP

SEQ ID NO: 350 - protein, 46 aa, Artificial
  Engineered binding domain
LAEAKVLANRELDKYGVSDFYKRLINRAKTVEGVNALISDILAALP

SEQ ID NO: 351 - protein, 46 aa, Artificial
  Engineered binding domain
LAEAKVLANRELDKYGVSDFYKRLINRAKTVEGVSALIQEILAALP

SEQ ID NO: 352 - protein, 46 aa, Artificial
  Engineered binding domain
LAEAKVLANRELDKYGVSDFYKRLINRAKTVEGVQSLIDHILAALP

SEQ ID NO: 353 - protein, 46 aa, Artificial
  Engineered binding domain
LAEAKVLANRELDKYGVSDFYKRLINRAKTVEGVDALICHILAALP

SEQ ID NO: 354 - protein, 46 aa, Artificial
  Engineered binding domain
LAEAKVLANRELDKYGVSDFYKRVINKAKTVEGVEALIADILAALP

SEQ ID NO: 355 - protein, 46 aa, Artificial
  Engineered binding domain
LAEAKVLANRELDKYGVSDFYKRLINRAKTVEGVETLIRDILAALP

SEQ ID NO: 356 - protein, 46 aa, Artificial
  Engineered binding domain
LAEAKVLANRELDKYGVSDFYKRLINRARTVEGVQTLITDILAALP

SEQ ID NO: 357 - protein, 46 aa, Artificial
  Engineered binding domain
LAEAKVLANRELDKYGVSDYYKRLINRAKTVEGVQALIAHILAALP

SEQ ID NO: 358 - protein, 46 aa, Artificial
  Engineered binding domain
LAEAKVLANRELDKYGVSDFYKRVINRAKTVEGVNSLINHILAALP

SEQ ID NO: 359 - protein, 46 aa, Artificial
  Engineered binding domain
LAEAKVLANRELDKYGVSDFYKRLINRARTVEGVDSLIRHILAALP

SEQ ID NO: 360 - protein, 46 aa, Artificial
  Engineered binding domain
LAEAKVLANRELDKYGVSDYYKRLINKAKTVEGVEALTLHILAALP

SEQ ID NO: 361 - protein, 46 aa, Artificial
  Engineered binding domain
LAEAKVLANRELDKYGVSDFYKRLINRAKTVEGVDALIAHILAALP

SEQ ID NO: 362 - protein, 46 aa, Artificial
  Engineered binding domain
LAEAKVLANRELDKYGVSDYYKRLINKAKTVEGVQALIAHILAALP

SEQ ID NO: 363 - protein, 46 aa, Artificial
  Engineered binding domain
LAEAKVLANRELDKYGVSDFYKNLINRAKTVEGVHALKGHILAALP

SEQ ID NO: 364 - protein, 46 aa, Artificial
  Engineered binding domain
LAEAKVLANRELDKYGVSDFYKNVINRAKTVEGVHALKDHILAALP

SEQ ID NO: 365 - protein, 46 aa, Artificial
  Engineered binding domain
LAEAKVLANRELDKYGVSDFYKNVINRAKTVEGVSALKHEILAALP

SEQ ID NO: 366 - protein, 46 aa, Artificial
  Engineered binding domain
LAEAKVLANRELDKYGVSDFYKRLINRAKTVEGVNTLKADILAALP

SEQ ID NO: 367 - protein, 46 aa, Artificial
  Engineered binding domain
LAEAKVLANRELDKYGVSDFYKNLINRAKTVEGVNTLKADILAALP

SEQ ID NO: 368 - protein, 46 aa, Artificial
  Engineered binding domain
LAEAKVLANRELDKYGVSDYYKNLINRAKTVEGVNSLKSHILAALP

SEQ ID NO: 369 - protein, 46 aa, Artificial
  Engineered binding domain
LAEAKVLANRELDKYGVSDFYKRLINRAKTVGGVQSLKSEILAALP

SEQ ID NO: 370 - protein, 46 aa, Artificial
  Engineered binding domain
LAEAKVLANRELDKYGVSDFYKNVINRAKTVEGVDSLKAEILAALP

SEQ ID NO: 371 - protein, 46 aa, Artificial
  Engineered binding domain
LAEAKVLANRELDKYGVSDFYKNLINRARTVEGVQTLKSDILAALP

SEQ ID NO: 372 - protein, 46 aa, Artificial
  Engineered binding domain
LAEAKVLANRELDKYGVSDFYKKFINKAKTVEGVETLKSEILAALP

SEQ ID NO: 373 - protein, 46 aa, Artificial
  Engineered binding domain
LAEAKVLANRELDKYGVSDFYKSLINRAKTVEGVHSLKDEILAALP

SEQ ID NO: 374 - protein, 46 aa, Artificial
  Engineered binding domain
LAEAKVLANRELDKYGVSDYYKNVINKAKTVEGVSSLKAEILAALP

SEQ ID NO: 375 - protein, 46 aa, Artificial
  Engineered binding domain
LAEAKVLANRELDKYGVSDFYKSLINRAKTVEGVDALKSHILAALP

SEQ ID NO: 376 - protein, 46 aa, Artificial
  Engineered binding domain
LAEAKVLANRELDKYGVSDFYKNLINKAKTVEGVSTLKHDILAALP

SEQ ID NO: 377 - protein, 46 aa, Artificial
  Engineered binding domain
LAEAKVLANRELDKYGVSDFYKNLINRAKTVEGVSTLKHDILAALP

SEQ ID NO: 378 - protein, 46 aa, Artificial
  Engineered binding domain
LAEAKVLANRELDKYGVSDFYKNLINRAKTVEGVQALKSEILAALP

SEQ ID NO: 379 - protein, 46 aa, Artificial
  Engineered binding domain
LAEAKVLANRELDKYGVSDYYKSLINKAKTVEGVDSLKVHILAALP

SEQ ID NO: 380 - protein, 46 aa, Artificial
  Engineered binding domain
LAEAKVLANRELDKYGVSDFYKNLINRAKTVEGVQSLKTEILAALP

SEQ ID NO: 381 - protein, 46 aa, Artificial
  Engineered binding domain
LAEAKVLANRELDKYGVSDYYKNLINRAKTVEGVDALKTHILAALP

SEQ ID NO: 382 - protein, 46 aa, Artificial
  Engineered binding domain
LAEAKVLANRELDKYGVSDFYKSMINRAKTVEGVDSLKTHILAALP

SEQ ID NO: 383 - protein, 46 aa, Artificial
  Engineered binding domain
LAEAKVLANRELDKYGVSDFYKNLINRAKTVEGVTTLKTDILAALP

SEQ ID NO: 384 - protein, 46 aa, Artificial
  Engineered binding domain
LAEAKVLANRELDKYGVSDFYKNLINRAKTVEGVESLKDHILAALP

SEQ ID NO: 385 - protein, 46 aa, Artificial
  Engineered binding domain
LAEAKVLANRELDKYGVSDFYKSYINRAKTVEGVHTLKGHILAALP

SEQ ID NO: 386 - protein, 46 aa, Artificial
  Engineered binding domain
LAEAKVLANRELDKYGVSDFYKNLINRAKTVEGVQTLKSDILAALP

SEQ ID NO: 387 - protein, 46 aa, Artificial
  Engineered binding domain
LAEAKVLANRELDKYGVSDFYKNLINRAKTVEGVNSLKSHILAALP

SEQ ID NO: 388 - protein, 46 aa, Artificial
  Engineered binding domain
LAEAKVLANRELDKYGVSDFYKNLINRAKTVEGVNTLKHDILAALP

SEQ ID NO: 389 - protein, 46 aa, Artificial
  Engineered binding domain
LAEAKVLANRELDKYGVSDFYKNLINRAKTVEGVESLKGEILAALP

SEQ ID NO: 390 - protein, 46 aa, Artificial
  Engineered binding domain
LAEAKVLANRELDKYGVSDFYKNLINKAKTVEGVHTLKHDILAALP

SEQ ID NO: 391 - protein, 46 aa, Artificial
  Engineered binding domain
LAEAKVLANRELDKYGVSDFYKNLINKAKTVEGVDALKVHILAALP

SEQ ID NO: 392 - protein, 46 aa, Artificial
  Engineered binding domain
LAEAKVLANRELDKYGVSDYYKRLINRAKTVEGVHALKAEILAALP

SEQ ID NO: 393 - protein, 46 aa, Artificial
  Engineered binding domain
LAEAKVLANRELDKYGVSDYYKNLINRARTVEGVDTLKHDILAALP

SEQ ID NO: 394 - protein, 46 aa, Artificial
  Engineered binding domain
LAEAKVLANRELDKYGVSDFYKKVINRARTVEGVQALKADILAALP

SEQ ID NO: 395 - protein, 46 aa, Artificial
  Engineered binding domain
LAEAKVLANRELDKYGVSDFYKNLINRAKTVEGVESLKADILAALP

SEQ ID NO: 396 - protein, 46 aa, Artificial
  Engineered binding domain
LAEAKVLANRELDKYGVSDYYKNLINKAKTVEGVDALKAHILAALP

SEQ ID NO: 397 - protein, 46 aa, Artificial
  Engineered binding domain
LAEAKVLANRELDKYGVSDYYKNLINRAKTVEGVESLKTHILAALP

SEQ ID NO: 398 - protein, 46 aa, Artificial
  Engineered binding domain
LAEAKVLANRELDKYGVSDFYKNLINRARTVEGVDSLKVEILAALP

SEQ ID NO: 399 - protein, 46 aa, Artificial
  Engineered binding domain
LAEAKVLANRELDKYGVSDFYKNVINRAKTVEGVSALKREILAALP

SEQ ID NO: 400 - protein, 46 aa, Artificial
  Engineered binding domain
LAEAKVLANRELDKYGVSDFYKNLINRAKTVEGVNALKSDILAALP

SEQ ID NO: 401 - protein, 46 aa, Artificial
  Engineered binding domain
LAEAKVLANRELDKYGVSDFYKNLINRAKTVEGVSALKQEILAALP

SEQ ID NO: 402 - protein, 46 aa, Artificial
  Engineered binding domain
LAEAKVLANRELDKYGVSDFYKNLINRAKTVEGVQSLKDHILAALP

SEQ ID NO: 403 - protein, 46 aa, Artificial
  Engineered binding domain
LAEAKVLANRELDKYGVSDFYKNLINRAKTVEGVDALKCHILAALP

SEQ ID NO: 404 - protein, 46 aa, Artificial
  Engineered binding domain
LAEAKVLANRELDKYGVSDYYKRLINKAKTVEGVNALKTHILAALP

SEQ ID NO: 405 - protein, 46 aa, Artificial
  Engineered binding domain
LAEAKVLANRELDKYGVSDFYKNVINKAKTVEGVEALKADILAALP

SEQ ID NO: 406 - protein, 46 aa, Artificial
  Engineered binding domain
LAEAKVLANRELDKYGVSDFYKNLINRAKTVEGVETLKRDILAALP

SEQ ID NO: 407 - protein, 46 aa, Artificial
  Engineered binding domain
LAEAKVLANRELDKYGVSDFYKNLINRARTVEGVQTLKTDILAALP

SEQ ID NO: 408 - protein, 46 aa, Artificial
  Engineered binding domain
LAEAKVLANRELDKYGVSDFYKRLINKAKTVEGVNALKHHILAALP

SEQ ID NO: 409 - protein, 46 aa, Artificial
  Engineered binding domain
LAEAKVLANRELDKYGVSDYYKNLINRAKTVEGVQALKAHILAALP

SEQ ID NO: 410 - protein, 46 aa, Artificial
  Engineered binding domain
LAEAKVLANRELDKYGVSDFYKNVINRAKTVEGVNSLKNHILAALP

SEQ ID NO: 411 - protein, 46 aa, Artificial
  Engineered binding domain
LAEAKVLANRELDKYGVSDFYKSLINRARTVEGVDSLKRHILAALP

SEQ ID NO: 412 - protein, 46 aa, Artificial
  Engineered binding domain
LAEAKVLANRELDKYGVSDYYKNLINKAKTVEGVEALKLHILAALP

SEQ ID NO: 413 - protein, 46 aa, Artificial
  Engineered binding domain
LAEAKVLANRELDKYGVSDFYKNLINRAKTVEGVDALKAHILAALP

SEQ ID NO: 414 - protein, 46 aa, Artificial
  Engineered binding domain
LAEAKVLANRELDKYGVSDYYKNLINKAKTVEGVQALKAHILAALP

SEQ ID NO: 415 - protein, 46 aa, Artificial
  Engineered binding domain
LAEAKVLANRELDKYGVSDFYKRLINRAKTVEGVHALKGHILAALP

SEQ ID NO: 416 - protein, 46 aa, Artificial
  Engineered binding domain
LAEAKVLANRELDKYGVSDFYKRVINRAKTVEGVHALKDHILAALP

SEQ ID NO: 417 - protein, 46 aa, Artificial; identical to SEQ ID NO: 315
  Engineered binding domain

SEQ ID NO: 418 - protein, 46 aa, Artificial
  Engineered binding domain
LAEAKVLANRELDKYGVSDFYKRVINRAKTVEGVSALKHEILAALP

SEQ ID NO: 419 - protein, 46 aa, Artificial
  Engineered binding domain
LAEAKVLANRELDKYGVSDYYKRLINRAKTVEGVNSLKSHILAALP

SEQ ID NO: 420 - protein, 46 aa, Artificial; identical to SEQ ID NO: 369
  Engineered binding domain

SEQ ID NO: 421 - protein, 46 aa, Artificial; identical to SEQ ID NO: 319
  Engineered binding domain

SEQ ID NO: 422 - protein, 46 aa, Artificial
  Engineered binding domain
LAEAKVLANRELDKYGVSDFYKRVINRAKTVEGVDSLKAEILAALP

SEQ ID NO: 423 - protein, 46 aa, Artificial
  Engineered binding domain
LAEAKVLANRELDKYGVSDFYKRLINRARTVEGVQTLKSDILAALP

SEQ ID NO: 424 - protein, 46 aa, Artificial
  Engineered binding domain
LAEAKVLANRELDKYGVSDFYKRFINKAKTVEGVETLKSEILAALP

SEQ ID NO: 425 - protein, 46 aa, Artificial
  Engineered binding domain
LAEAKVLANRELDKYGVSDFYKRLINRAKTVEGVHSLKDEILAALP

SEQ ID NO: 426 - protein, 46 aa, Artificial
  Engineered binding domain
LAEAKVLANRELDKYGVSDYYKRVINKAKTVEGVSSLKAEILAALP

SEQ ID NO: 427 - protein, 46 aa, Artificial
  Engineered binding domain
LAEAKVLANRELDKYGVSDFYKRLINRAKTVEGVDALKSHILAALP

SEQ ID NO: 428 - protein, 46 aa, Artificial
  Engineered binding domain
LAEAKVLANRELDKYGVSDFYKRLINKAKTVEGVSTLKHDILAALP

SEQ ID NO: 429 - protein, 46 aa, Artificial
  Engineered binding domain
LAEAKVLANRELDKYGVSDFYKRLINRAKTVEGVSTLKHDILAALP

SEQ ID NO: 430 - protein, 46 aa, Artificial
  Engineered binding domain
LAEAKVLANRELDKYGVSDFYKRLINRAKTVEGVQALKSEILAALP

SEQ ID NO: 431 - protein, 46 aa, Artificial
  Engineered binding domain
LAEAKVLANRELDKYGVSDYYKRLINKAKTVEGVDSLKVHILAALP

SEQ ID NO: 432 - protein, 46 aa, Artificial
  Engineered binding domain
LAEAKVLANRELDKYGVSDFYKRLINRAKTVEGVQSLKTEILAALP

SEQ ID NO: 433 - protein, 46 aa, Artificial
  Engineered binding domain
LAEAKVLANRELDKYGVSDYYKRLINRAKTVEGVDALKTHILAALP

SEQ ID NO: 434 - protein, 46 aa, Artificial
  Engineered binding domain
LAEAKVLANRELDKYGVSDFYKRMINRAKTVEGVDSLKTHILAALP

SEQ ID NO: 435 - protein, 46 aa, Artificial
  Engineered binding domain
LAEAKVLANRELDKYGVSDFYKRLINRAKTVEGVTTLKTDILAALP

SEQ ID NO: 436 - protein, 46 aa, Artificial
  Engineered binding domain
LAEAKVLANRELDKYGVSDFYKRLINRAKTVEGVESLKDHILAALP

SEQ ID NO: 437 - protein, 46 aa, Artificial
  Engineered binding domain
LAEAKVLANRELDKYGVSDFYKRYINRAKTVEGVHTLKGHILAALP

SEQ ID NO: 438 - protein, 46 aa, Artificial
  Engineered binding domain
LAEAKVLANRELDKYGVSDFYKRLINRAKTVEGVQTLKSDILAALP

SEQ ID NO: 439 - protein, 46 aa, Artificial
  Engineered binding domain
LAEAKVLANRELDKYGVSDFYKRLINRAKTVEGVNSLKSHILAALP

SEQ ID NO: 440 - protein, 46 aa, Artificial
  Engineered binding domain
LAEAKVLANRELDKYGVSDFYKRLINRAKTVEGVNTLKHDILAALP

SEQ ID NO: 441 - protein, 46 aa, Artificial
  Engineered binding domain
LAEAKVLANRELDKYGVSDFYKRLINRAKTVEGVESLKGEILAALP

SEQ ID NO: 442 - protein, 46 aa, Artificial
  Engineered binding domain
LAEAKVLANRELDKYGVSDFYKRLINKAKTVEGVHTLKHDILAALP

SEQ ID NO: 443 - protein, 46 aa, Artificial; identical to SEQ ID NO: 341
  Engineered binding domain

SEQ ID NO: 444 - protein, 46 aa, Artificial
  Engineered binding domain
LAEAKVLANRELDKYGVSDFYKRLINKAKTVEGVDALKVHILAALP

SEQ ID NO: 445 - protein, 46 aa, Artificial; identical to SEQ ID NO: 392
  Engineered binding domain

SEQ ID NO: 446 - protein, 46 aa, Artificial
  Engineered binding domain
LAEAKVLANRELDKYGVSDYYKRLINRARTVEGVDTLKHDILAALP

SEQ ID NO: 447 - protein, 46 aa, Artificial
  Engineered binding domain
LAEAKVLANRELDKYGVSDFYKRVINRARTVEGVQALKADILAALP

SEQ ID NO: 448 - protein, 46 aa, Artificial
  Engineered binding domain
LAEAKVLANRELDKYGVSDFYKRLINRAKTVEGVESLKADILAALP

SEQ ID NO: 449 - protein, 46 aa, Artificial
  Engineered binding domain
LAEAKVLANRELDKYGVSDYYKRLINKAKTVEGVDALKAHILAALP

SEQ ID NO: 450 - protein, 46 aa, Artificial
  Engineered binding domain
LAEAKVLANRELDKYGVSDYYKRLINRAKTVEGVESLKTHILAALP

SEQ ID NO: 451 - protein, 46 aa, Artificial
  Engineered binding domain
LAEAKVLANRELDKYGVSDFYKRLINRARTVEGVDSLKVEILAALP

SEQ ID NO: 452 - protein, 46 aa, Artificial
  Engineered binding domain
LAEAKVLANRELDKYGVSDFYKRVINRAKTVEGVSALKREILAALP

SEQ ID NO: 453 - protein, 46 aa, Artificial
  Engineered binding domain
LAEAKVLANRELDKYGVSDFYKRLINRAKTVEGVNALKSDILAALP

SEQ ID NO: 454 - protein, 46 aa, Artificial
  Engineered binding domain
LAEAKVLANRELDKYGVSDFYKRLINRAKTVEGVSALKQEILAALP

SEQ ID NO: 455 - protein, 46 aa, Artificial
  Engineered binding domain
LAEAKVLANRELDKYGVSDFYKRLINRAKTVEGVQSLKDHILAALP

SEQ ID NO: 456 - protein, 46 aa, Artificial
  Engineered binding domain
LAEAKVLANRELDKYGVSDFYKRLINRAKTVEGVDALKCHILAALP

SEQ ID NO: 457 - protein, 46 aa, Artificial; identical to SEQ ID NO: 404
  Engineered binding domain

SEQ ID NO: 458 - protein, 46 aa, Artificial
  Engineered binding domain
LAEAKVLANRELDKYGVSDFYKRVINKAKTVEGVEALKADILAALP

SEQ ID NO: 459 - protein, 46 aa, Artificial
  Engineered binding domain
LAEAKVLANRELDKYGVSDFYKRLINRAKTVEGVETLKRDILAALP

SEQ ID NO: 460 - protein, 46 aa, Artificial
  Engineered binding domain
LAEAKVLANRELDKYGVSDFYKRLINRARTVEGVQTLKTDILAALP

SEQ ID NO: 461 - protein, 46 aa, Artificial; identical to SEQ ID NO: 408
  Engineered binding domain

SEQ ID NO: 462 - protein, 46 aa, Artificial
  Engineered binding domain
LAEAKVLANRELDKYGVSDYYKRLINRAKTVEGVQALKAHILAALP

SEQ ID NO: 463 - protein, 46 aa, Artificial
  Engineered binding domain
LAEAKVLANRELDKYGVSDFYKRVINRAKTVEGVNSLKNHILAALP

SEQ ID NO: 464 - protein, 46 aa, Artificial
  Engineered binding domain
LAEAKVLANRELDKYGVSDFYKRLINRARTVEGVDSLKRHILAALP

SEQ ID NO: 465 - protein, 46 aa, Artificial
  Engineered binding domain
LAEAKVLANRELDKYGVSDYYKRLINKAKTVEGVEALKLHILAALP

SEQ ID NO: 466 - protein, 46 aa, Artificial
  Engineered binding domain
LAEAKVLANRELDKYGVSDFYKRLINRAKTVEGVDALKAHILAALP

SEQ ID NO: 467 - protein, 46 aa, Artificial
  Engineered binding domain
LAEAKVLANRELDKYGVSDYYKRLINKAKTVEGVQALKAHILAALP

SEQ ID NO: 468 - protein, 46 aa, Artificial
  Engineered binding domain
LAEAKVLANRELDKYGVSDFYKRVINRAKTVEGVSALKHHILAALP

SEQ ID NO: 469 - protein, 46 aa, Artificial
  Engineered binding domain
LAEAKVLANRELDKYGVSDFYKRLINRAKTVEGVNTLKAHILAALP

SEQ ID NO: 470 - protein, 46 aa, Artificial
  Engineered binding domain
LAEAKVLANRELDKYGVSDFYKRLINRAKTVGGVQSLKSHILAALP

SEQ ID NO: 471 - protein, 46 aa, Artificial
  Engineered binding domain
LAEAKVLANRELDKYGVSDFYKRVINRAKTVEGVDSLKAHILAALP

SEQ ID NO: 472 - protein, 46 aa, Artificial
  Engineered binding domain
LAEAKVLANRELDKYGVSDFYKRLINRARTVEGVQTLKSHILAALP

SEQ ID NO: 473 - protein, 46 aa, Artificial
  Engineered binding domain
LAEAKVLANRELDKYGVSDFYKRFINKAKTVEGVETLKSHILAALP

SEQ ID NO: 474 - protein, 46 aa, Artificial
  Engineered binding domain
LAEAKVLANRELDKYGVSDFYKRLINRAKTVEGVHSLKDHILAALP

SEQ ID NO: 475 - protein, 46 aa, Artificial
  Engineered binding domain
LAEAKVLANRELDKYGVSDYYKRVINKAKTVEGVSSLKAHILAALP

SEQ ID NO: 476 - protein, 46 aa, Artificial
  Engineered binding domain
LAEAKVLANRELDKYGVSDFYKRLINKAKTVEGVSTLKHHILAALP

SEQ ID NO: 477 - protein, 46 aa, Artificial
  Engineered binding domain
LAEAKVLANRELDKYGVSDFYKRLINRAKTVEGVSTLKHHILAALP

SEQ ID NO: 478 - protein, 46 aa, Artificial
  Engineered binding domain
LAEAKVLANRELDKYGVSDFYKRLINRAKTVEGVQALKSHILAALP

SEQ ID NO: 479 - protein, 46 aa, Artificial
  Engineered binding domain
LAEAKVLANRELDKYGVSDFYKRLINRAKTVEGVQSLKTHILAALP

SEQ ID NO: 480 - protein, 46 aa, Artificial
  Engineered binding domain
LAEAKVLANRELDKYGVSDFYKRLINRAKTVEGVTTLKTHILAALP

SEQ ID NO: 481 - protein, 46 aa, Artificial
  Engineered binding domain
LAEAKVLANRELDKYGVSDFYKRLINRAKTVEGVQTLKSHILAALP

SEQ ID NO: 482 - protein, 46 aa, Artificial
  Engineered binding domain
LAEAKVLANRELDKYGVSDFYKRLINRAKTVEGVNTLKHHILAALP

SEQ ID NO: 483 - protein, 46 aa, Artificial
  Engineered binding domain
LAEAKVLANRELDKYGVSDFYKRLINRAKTVEGVESLKGHILAALP

SEQ ID NO: 484 - protein, 46 aa, Artificial
  Engineered binding domain
LAEAKVLANRELDKYGVSDFYKRLINKAKTVEGVHTLKHHILAALP

SEQ ID NO: 485 - protein, 46 aa, Artificial
  Engineered binding domain
LAEAKVLANRELDKYGVSDYYKRLINRAKTVEGVHALKAHILAALP

SEQ ID NO: 486 - protein, 46 aa, Artificial
  Engineered binding domain
LAEAKVLANRELDKYGVSDYYKRLINRARTVEGVDTLKHHILAALP

SEQ ID NO: 487 - protein, 46 aa, Artificial
  Engineered binding domain
LAEAKVLANRELDKYGVSDFYKRVINRARTVEGVQALKAHILAALP

SEQ ID NO: 488 - protein, 46 aa, Artificial
  Engineered binding domain
LAEAKVLANRELDKYGVSDFYKRLINRAKTVEGVESLKAHILAALP

SEQ ID NO: 489 - protein, 46 aa, Artificial
  Engineered binding domain
LAEAKVLANRELDKYGVSDFYKRLINRARTVEGVDSLKVHILAALP

SEQ ID NO: 490 - protein, 46 aa, Artificial
  Engineered binding domain
LAEAKVLANRELDKYGVSDFYKRVINRAKTVEGVSALKRHILAALP

SEQ ID NO: 491 - protein, 46 aa, Artificial
  Engineered binding domain
LAEAKVLANRELDKYGVSDFYKRLINRAKTVEGVNALKSHILAALP

SEQ ID NO: 492 - protein, 46 aa, Artificial
  Engineered binding domain
LAEAKVLANRELDKYGVSDFYKRLINRAKTVEGVSALKQHILAALP

SEQ ID NO: 493 - protein, 46 aa, Artificial
  Engineered binding domain
LAEAKVLANRELDKYGVSDFYKRVINKAKTVEGVEALKAHILAALP

SEQ ID NO: 494 - protein, 46 aa, Artificial
  Engineered binding domain
LAEAKVLANRELDKYGVSDFYKRLINRAKTVEGVETLKRHILAALP

SEQ ID NO: 495 - protein, 46 aa, Artificial
  Engineered binding domain
LAEAKVLANRELDKYGVSDFYKRLINRARTVEGVQTLKTHILAALP

SEQ ID NO: 496 - protein, 46 aa, Artificial
  Engineered binding domain
LAEAKVLANRELDKYGVSDFYKRLINKAKTVEGVEALKLHILAALP

SEQ ID NO: 497 - protein, 46 aa, Artificial
  Engineered binding domain
LAEAKVLANRELDKYGVSDYYKNLINRARTVEGVEALKLHILAALP

SEQ ID NO: 498 - protein, 46 aa, Artificial
  Engineered binding domain
LAEAKVLANRELDKYGVSDYYKNIINRAKTVEGVEALKLHILAALP

SEQ ID NO: 499 - protein, 46 aa, Artificial
  Engineered binding domain
LAEAKVLANRELDKYGVSDFYKNLINRAKTVEGVEALKLHILAALP

SEQ ID NO: 500 - protein, 46 aa, Artificial
  Engineered binding domain
LAEAKVLANRELDKYGVSDFYKNVINKAKTVEGVEALKLHILAALP

SEQ ID NO: 501 - protein, 46 aa, Artificial
  Engineered binding domain
LAEAKVLANRELDKYGVSDYYKNLINRAKTVEGVEALKLHILAALP

SEQ ID NO: 502 - protein, 46 aa, Artificial
  Engineered binding domain
LAEAKVLANRELDKYGVSDYYKNLINRARTVEGVHALIDHILAALP

SEQ ID NO: 503 - protein, 46 aa, Artificial
  Engineered binding domain
LAEAKVLALRELDKYGVSDFYKRLINKAKTVEGVEALKLHILAALP

SEQ ID NO: 504 - protein, 46 aa, Artificial
  Engineered binding domain
LAEAKVLALRELDKYGVSDYYKRLISKAKTVEGVKALISEILAALP

SEQ ID NO: 505 - protein, 46 aa, Artificial
  Engineered binding domain
LAEAKVLAIRELDKYGVSDFYKRLINKAKTVEGVEALKLHILAALP

SEQ ID NO: 506 - protein, 46 aa, Artificial
  Engineered binding domain
LAEAKVLAIRELDKYGVSDYYKRLISKAKTVEGVKALISEILAALP

SEQ ID NO: 507 - protein, 46 aa, Artificial
  Engineered binding domain
LAEAKVLAIKELDKYGVSDYYKRLISKAKTVEGVKALISEILAALP

SEQ ID NO: 508 - protein, 46 aa, Artificial
  Engineered binding domain
LAEAKELANRELDKYGVSDYYKRLISKAKTVEGVKALISEILAALP

SEQ ID NO: 509 - protein, 46 aa, Artificial
  Engineered binding domain
LAEAKVDANRELDKYGVSDYYKRLISKAKTVEGVKALISEILAALP

SEQ ID NO: 510 - protein, 46 aa, Artificial
  Engineered binding domain
LAEAKEDANRELDKYGVSDYYKRLISKAKTVEGVKALISEILAALP

SEQ ID NO: 511 - protein, 46 aa, Artificial
  Engineered binding domain
LAEAKEDAIKELDKYGVSDYYKRLISKAKTVEGVKALISEILAALP

SEQ ID NO: 512 - protein, 46 aa, Artificial
  Engineered binding domain
LAEAKVLALKELDKYGVSDYYKRLISKAKTVEGVKALISEILAALP

SEQ ID NO: 513 - protein, 46 aa, Artificial
  Engineered binding domain
LAEAKELAIKELDKYGVSDYYKRLISKAKTVEGVKALISEILAALP

SEQ ID NO: 514 - protein, 46 aa, Artificial
  Engineered binding domain
LAEAKVDAIKELDKYGVSDYYKRLISKAKTVEGVKALISEILAALP

SEQ ID NO: 515 - protein, 46 aa, Streptococcus sp. G148
LAEAKVLANRELDKYGVSDYYKNLINNAKTVEGVKALIDEILAALP

SEQ ID NO: 516 - protein, 46 aa, Artificial
  Engineered binding domain
LASAKEAANAELDAYGVSDFYKRLIDKAKTVEGVEALKDAILAALP

SEQ ID NO: 517 - protein, 46 aa, Artificial
  Engineered binding domain
LASAKEAANSELDAYGVSDFYKRLIDKAKTVEGVEALKDAILAALP

SEQ ID NO: 518 - protein, 46 aa, Artificial
  Engineered binding domain
LASAKESANSELDAYGVSDFYKRLIDKAKTVEGVEALKDAILAALP

SEQ ID NO: 519 - protein, 46 aa, Artificial
  Engineered binding domain
LASAKESANAELDAYGVSDFYKRLIDKAKTVEGVEALKDAILAALP

SEQ ID NO: 520 - protein, 46 aa, Artificial
  Engineered binding domain
LASAKSAANAELDAYGVSDFYKRLIDKAKTVEGVEALKDAILAALP

SEQ ID NO: 521 - protein, 46 aa, Artificial
  Engineered binding domain
LASAKSAANSELDAYGVSDFYKRLIDKAKTVEGVEALKDAILAALP

SEQ ID NO: 522 - protein, 46 aa, Artificial
  Engineered binding domain
LASAKEAANAELDSYGVSDFYKRLIDKAKTVEGVEALKDAILAALP

SEQ ID NO: 523 - protein, 46 aa, Artificial
  Engineered binding domain
LASAKEAANSELDSYGVSDFYKRLIDKAKTVEGVEALKDAILAALP

SEQ ID NO: 524 - protein, 46 aa, Artificial
  Engineered binding domain
LASAKESANSELDSYGVSDFYKRLIDKAKTVEGVEALKDAILAALP

SEQ ID NO: 525 - protein, 46 aa, Artificial
  Engineered binding domain
LASAKESANAELDSYGVSDFYKRLIDKAKTVEGVEALKDAILAALP

SEQ ID NO: 526 - protein, 46 aa, Artificial
  Engineered binding domain
LASAKSAANAELDSYGVSDFYKRLIDKAKTVEGVEALKDAILAALP

SEQ ID NO: 527 - protein, 46 aa, Artificial
  Engineered binding domain
LASAKSAANSELDSYGVSDFYKRLIDKAKTVEGVEALKDAILAALP

SEQ ID NO: 528 - protein, 46 aa, Artificial
  Engineered binding domain
LAEAKEAANAELDSYGVSDFYKRLIDKAKTVEGVEALKDAILAALP

SEQ ID NO: 529 - protein, 46 aa, Artificial
  Engineered binding domain
LAEAKEAANSELDSYGVSDFYKRLIDKAKTVEGVEALKDAILAALP

SEQ ID NO: 530 - protein, 46 aa, Artificial
  Engineered binding domain
LAEAKESANSELDSYGVSDFYKRLIDKAKTVEGVEALKDAILAALP

SEQ ID NO: 531 - protein, 46 aa, Artificial
  Engineered binding domain
LAEAKESANAELDSYGVSDFYKRLIDKAKTVEGVEALKDAILAALP

SEQ ID NO: 532 - protein, 46 aa, Artificial
  Engineered binding domain
LAEAKSAANAELDSYGVSDFYKRLIDKAKTVEGVEALKDAILAALP

SEQ ID NO: 533 - protein, 46 aa, Artificial
  Engineered binding domain
LAEAKSAANSELDSYGVSDFYKRLIDKAKTVEGVEALKDAILAALP

SEQ ID NO: 534 - protein, 46 aa, Artificial
  Engineered binding domain
LAEAKEAANAELDAYGVSDFYKRLIDKAKTVEGVEALKDAILAALP

SEQ ID NO: 535 - protein, 46 aa, Artificial
  Engineered binding domain
LAEAKEAANSELDAYGVSDFYKRLIDKAKTVEGVEALKDAILAALP

SEQ ID NO: 536 - protein, 46 aa, Artificial
  Engineered binding domain
LAEAKESANSELDAYGVSDFYKRLIDKAKTVEGVEALKDAILAALP

SEQ ID NO: 537 - protein, 46 aa, Artificial
  Engineered binding domain
LAEAKESANAELDAYGVSDFYKRLIDKAKTVEGVEALKDAILAALP

SEQ ID NO: 538 - protein, 46 aa, Artificial
  Engineered binding domain
LAEAKSAANAELDAYGVSDFYKRLIDKAKTVEGVEALKDAILAALP

SEQ ID NO: 539 - protein, 46 aa, Artificial
  Engineered binding domain
LAEAKSAANSELDAYGVSDFYKRLIDKAKTVEGVEALKDAILAALP

SEQ ID NO: 540 - protein, 46 aa, Artificial
  Engineered binding domain
LAQAKEAANAELDAYGVSDFYKRLIDKAKTVEGVEALKDAILAALP

SEQ ID NO: 541 - protein, 46 aa, Artificial
  Engineered binding domain
LAQAKEAANSELDAYGVSDFYKRLIDKAKTVEGVEALKDAILAALP

SEQ ID NO: 542 - protein, 46 aa, Artificial
  Engineered binding domain
LAQAKESANSELDAYGVSDFYKRLIDKAKTVEGVEALKDAILAALP

SEQ ID NO: 543 - protein, 46 aa, Artificial
  Engineered binding domain
LAQAKESANAELDAYGVSDFYKRLIDKAKTVEGVEALKDAILAALP

SEQ ID NO: 544 - protein, 46 aa, Artificial
  Engineered binding domain
LAQAKSAANAELDAYGVSDFYKRLIDKAKTVEGVEALKDAILAALP

SEQ ID NO: 545 - protein, 46 aa, Artificial
  Engineered binding domain
LAQAKSAANSELDAYGVSDFYKRLIDKAKTVEGVEALKDAILAALP

SEQ ID NO: 546 - protein, 46 aa, Artificial
  Engineered binding domain
LAQAKEAANAELDSYGVSDFYKRLIDKAKTVEGVEALKDAILAALP

SEQ ID NO: 547 - protein, 46 aa, Artificial
  Engineered binding domain
LAQAKEAANSELDSYGVSDFYKRLIDKAKTVEGVEALKDAILAALP

SEQ ID NO: 548 - protein, 46 aa, Artificial
  Engineered binding domain
LAQAKESANSELDSYGVSDFYKRLIDKAKTVEGVEALKDAILAALP

SEQ ID NO: 549 - protein, 46 aa, Artificial
  Engineered binding domain
LAQAKESANAELDSYGVSDFYKRLIDKAKTVEGVEALKDAILAALP

SEQ ID NO: 550 - protein, 46 aa, Artificial
  Engineered binding domain
LAQAKSAANAELDSYGVSDFYKRLIDKAKTVEGVEALKDAILAALP

SEQ ID NO: 551 - protein, 46 aa, Artificial
  Engineered binding domain
LAQAKSAANSELDSYGVSDFYKRLIDKAKTVEGVEALKDAILAALP

SEQ ID NO: 552 - protein, 46 aa, Artificial
  Engineered binding domain
LASAKEAANAELDCYGVSDFYKRLIDKAKTVEGVEALKDAILAALP

SEQ ID NO: 553 - protein, 46 aa, Artificial
  Engineered binding domain
LASAKEAANSELDCYGVSDFYKRLIDKAKTVEGVEALKDAILAALP

SEQ ID NO: 554 - protein, 46 aa, Artificial
  Engineered binding domain
LASAKESANSELDCYGVSDFYKRLIDKAKTVEGVEALKDAILAALP

SEQ ID NO: 555 - protein, 46 aa, Artificial
  Engineered binding domain
LASAKESANAELDCYGVSDFYKRLIDKAKTVEGVEALKDAILAALP

SEQ ID NO: 556 - protein, 46 aa, Artificial
  Engineered binding domain
LASAKSAANAELDCYGVSDFYKRLIDKAKTVEGVEALKDAILAALP

SEQ ID NO: 557 - protein, 46 aa, Artificial
  Engineered binding domain
LASAKSAANSELDCYGVSDFYKRLIDKAKTVEGVEALKDAILAALP

SEQ ID NO: 558 - protein, 46 aa, Artificial
  Engineered binding domain
LASAKEAANAELDKYGVSDFYKRLIDKAKTVEGVEALKDAILAALP

SEQ ID NO: 559 - protein, 46 aa, Artificial
  Engineered binding domain
LASAKEAANSELDKYGVSDFYKRLIDKAKTVEGVEALKDAILAALP

SEQ ID NO: 560 - protein, 46 aa, Artificial
  Engineered binding domain
LASAKESANSELDKYGVSDFYKRLIDKAKTVEGVEALKDAILAALP

SEQ ID NO: 561 - protein, 46 aa, Artificial
  Engineered binding domain
LASAKESANAELDKYGVSDFYKRLIDKAKTVEGVEALKDAILAALP

SEQ ID NO: 562 - protein, 46 aa, Artificial
  Engineered binding domain
LASAKSAANAELDKYGVSDFYKRLIDKAKTVEGVEALKDAILAALP

SEQ ID NO: 563 - protein, 46 aa, Artificial
  Engineered binding domain
LASAKSAANSELDKYGVSDFYKRLIDKAKTVEGVEALKDAILAALP

SEQ ID NO: 564 - protein, 46 aa, Artificial
  Engineered binding domain
LAEAKEAANAELDCYGVSDFYKRLIDKAKTVEGVEALKDAILAALP

SEQ ID NO: 565 - protein, 46 aa, Artificial
  Engineered binding domain
LAEAKEAANSELDCYGVSDFYKRLIDKAKTVEGVEALKDAILAALP

SEQ ID NO: 566 - protein, 46 aa, Artificial
  Engineered binding domain
LAEAKESANSELDCYGVSDFYKRLIDKAKTVEGVEALKDAILAALP

SEQ ID NO: 567 - protein, 46 aa, Artificial
  Engineered binding domain
LAEAKESANAELDCYGVSDFYKRLIDKAKTVEGVEALKDAILAALP

SEQ ID NO: 568 - protein, 46 aa, Artificial
  Engineered binding domain
LAEAKSAANAELDCYGVSDFYKRLIDKAKTVEGVEALKDAILAALP

SEQ ID NO: 569 - protein, 46 aa, Artificial
  Engineered binding domain
LAEAKSAANSELDCYGVSDFYKRLIDKAKTVEGVEALKDAILAALP

SEQ ID NO: 570 - protein, 46 aa, Artificial
  Engineered binding domain
LAEAKEAANAELDKYGVSDFYKRLIDKAKTVEGVEALKDAILAALP

SEQ ID NO: 571 - protein, 46 aa, Artificial
  Engineered binding domain
LAEAKEAANSELDKYGVSDFYKRLIDKAKTVEGVEALKDAILAALP

SEQ ID NO: 572 - protein, 46 aa, Artificial
  Engineered binding domain
LAEAKESANSELDKYGVSDFYKRLIDKAKTVEGVEALKDAILAALP

SEQ ID NO: 573 - protein, 46 aa, Artificial
  Engineered binding domain
LAEAKESANAELDKYGVSDFYKRLIDKAKTVEGVEALKDAILAALP

SEQ ID NO: 574 - protein, 46 aa, Artificial
  Engineered binding domain
LAEAKSAANAELDKYGVSDFYKRLIDKAKTVEGVEALKDAILAALP

SEQ ID NO: 575 - protein, 46 aa, Artificial
  Engineered binding domain
LAEAKSAANSELDKYGVSDFYKRLIDKAKTVEGVEALKDAILAALP

SEQ ID NO: 576 - protein, 46 aa, Artificial
  Engineered binding domain
LAQAKEAANAELDCYGVSDFYKRLIDKAKTVEGVEALKDAILAALP

SEQ ID NO: 577 - protein, 46 aa, Artificial
  Engineered binding domain
LAQAKEAANSELDCYGVSDFYKRLIDKAKTVEGVEALKDAILAALP

SEQ ID NO: 578 - protein, 46 aa, Artificial
  Engineered binding domain
LAQAKESANSELDCYGVSDFYKRLIDKAKTVEGVEALKDAILAALP

SEQ ID NO: 579 - protein, 46 aa, Artificial
  Engineered binding domain
LAQAKESANAELDCYGVSDFYKRLIDKAKTVEGVEALKDAILAALP

SEQ ID NO: 580 - protein, 46 aa, Artificial
  Engineered binding domain
LAQAKSAANAELDCYGVSDFYKRLIDKAKTVEGVEALKDAILAALP

SEQ ID NO: 581 - protein, 46 aa, Artificial
  Engineered binding domain
LAQAKSAANSELDCYGVSDFYKRLIDKAKTVEGVEALKDAILAALP

SEQ ID NO: 582 - protein, 46 aa, Artificial
  Engineered binding domain
LAQAKEAANAELDKYGVSDFYKRLIDKAKTVEGVEALKDAILAALP

SEQ ID NO: 583 - protein, 46 aa, Artificial
  Engineered binding domain
LAQAKEAANSELDKYGVSDFYKRLIDKAKTVEGVEALKDAILAALP

SEQ ID NO: 584 - protein, 46 aa, Artificial
  Engineered binding domain
LAQAKESANSELDKYGVSDFYKRLIDKAKTVEGVEALKDAILAALP

SEQ ID NO: 585 - protein, 46 aa, Artificial
  Engineered binding domain
LAQAKESANAELDKYGVSDFYKRLIDKAKTVEGVEALKDAILAALP

SEQ ID NO: 586 - protein, 46 aa, Artificial
  Engineered binding domain
LAQAKSAANAELDKYGVSDFYKRLIDKAKTVEGVEALKDAILAALP

SEQ ID NO: 587 - protein, 46 aa, Artificial
  Engineered binding domain
LAQAKSAANSELDKYGVSDFYKRLIDKAKTVEGVEALKDAILAALP

SEQ ID NO: 588 - protein, 46 aa, Artificial
  Engineered binding domain
LACAKEAANAELDCYGVSDFYKRLIDKAKTVEGVEALKDAILAALP

SEQ ID NO: 589 - protein, 46 aa, Artificial
  Engineered binding domain
LACAKEAANSELDCYGVSDFYKRLIDKAKTVEGVEALKDAILAALP

SEQ ID NO: 590 - protein, 46 aa, Artificial
  Engineered binding domain
LACAKESANSELDCYGVSDFYKRLIDKAKTVEGVEALKDAILAALP

SEQ ID NO: 591 - protein, 46 aa, Artificial
  Engineered binding domain
LACAKESANAELDCYGVSDFYKRLIDKAKTVEGVEALKDAILAALP

SEQ ID NO: 592 - protein, 46 aa, Artificial
  Engineered binding domain
LACAKSAANAELDCYGVSDFYKRLIDKAKTVEGVEALKDAILAALP

SEQ ID NO: 593 - protein, 46 aa, Artificial
  Engineered binding domain
LACAKSAANSELDCYGVSDFYKRLIDKAKTVEGVEALKDAILAALP

SEQ ID NO: 594 - protein, 46 aa, Artificial
  Engineered binding domain
LACAKEAANAELDKYGVSDFYKRLIDKAKTVEGVEALKDAILAALP

SEQ ID NO: 595 - protein, 46 aa, Artificial
  Engineered binding domain
LACAKEAANSELDKYGVSDFYKRLIDKAKTVEGVEALKDAILAALP

SEQ ID NO: 596 - protein, 46 aa, Artificial
  Engineered binding domain
LACAKESANSELDKYGVSDFYKRLIDKAKTVEGVEALKDAILAALP

SEQ ID NO: 597 - protein, 46 aa, Artificial
  Engineered binding domain
LACAKESANAELDKYGVSDFYKRLIDKAKTVEGVEALKDAILAALP

SEQ ID NO: 598 - protein, 46 aa, Artificial
  Engineered binding domain
LACAKSAANAELDKYGVSDFYKRLIDKAKTVEGVEALKDAILAALP

SEQ ID NO: 599 - protein, 46 aa, Artificial
  Engineered binding domain
LACAKSAANSELDKYGVSDFYKRLIDKAKTVEGVEALKDAILAALP

SEQ ID NO: 600 - protein, 46 aa, Artificial
  Engineered binding domain
LACAKEAANAELDSYGVSDFYKRLIDKAKTVEGVEALKDAILAALP

SEQ ID NO: 601 - protein, 46 aa, Artificial
  Engineered binding domain
LACAKEAANSELDSYGVSDFYKRLIDKAKTVEGVEALKDAILAALP

SEQ ID NO: 602 - protein, 46 aa, Artificial
  Engineered binding domain
LACAKESANSELDSYGVSDFYKRLIDKAKTVEGVEALKDAILAALP

SEQ ID NO: 603 - protein, 46 aa, Artificial
  Engineered binding domain
LACAKESANAELDSYGVSDFYKRLIDKAKTVEGVEALKDAILAALP

SEQ ID NO: 604 - protein, 46 aa, Artificial
  Engineered binding domain
LACAKSAANAELDSYGVSDFYKRLIDKAKTVEGVEALKDAILAALP

SEQ ID NO: 605 - protein, 46 aa, Artificial
  Engineered binding domain
LACAKSAANSELDSYGVSDFYKRLIDKAKTVEGVEALKDAILAALP

SEQ ID NO: 606 - protein, 46 aa, Artificial
  Engineered binding domain
LACAKEAANAELDAYGVSDFYKRLIDKAKTVEGVEALKDAILAALP

SEQ ID NO: 607 - protein, 46 aa, Artificial
  Engineered binding domain
LACAKEAANSELDAYGVSDFYKRLIDKAKTVEGVEALKDAILAALP

SEQ ID NO: 608 - protein, 46 aa, Artificial
  Engineered binding domain
LACAKESANSELDAYGVSDFYKRLIDKAKTVEGVEALKDAILAALP

SEQ ID NO: 609 - protein, 46 aa, Artificial
  Engineered binding domain
LACAKESANAELDAYGVSDFYKRLIDKAKTVEGVEALKDAILAALP

SEQ ID NO: 610 - protein, 46 aa, Artificial
  Engineered binding domain
LACAKSAANAELDAYGVSDFYKRLIDKAKTVEGVEALKDAILAALP

SEQ ID NO: 611 - protein, 46 aa, Artificial
  Engineered binding domain
LACAKSAANSELDAYGVSDFYKRLIDKAKTVEGVEALKDAILAALP

SEQ ID NO: 612 - protein, 46 aa, Artificial
  Engineered binding domain
LAQAKCAANAELDAYGVSDFYKRLIDKAKTVEGVEALKDAILAALP

SEQ ID NO: 613 - protein, 46 aa, Artificial
  Engineered binding domain
LAQAKCAANSELDAYGVSDFYKRLIDKAKTVEGVEALKDAILAALP

SEQ ID NO: 614 - protein, 46 aa, Artificial
  Engineered binding domain
LAQAKCSANSELDAYGVSDFYKRLIDKAKTVEGVEALKDAILAALP

SEQ ID NO: 615 - protein, 46 aa, Artificial
  Engineered binding domain
LAQAKCSANAELDAYGVSDFYKRLIDKAKTVEGVEALKDAILAALP

SEQ ID NO: 616 - protein, 46 aa, Artificial
  Engineered binding domain
LAQAKCAANAELDCYGVSDFYKRLIDKAKTVEGVEALKDAILAALP

SEQ ID NO: 617 - protein, 46 aa, Artificial
  Engineered binding domain
LAQAKCAANSELDCYGVSDFYKRLIDKAKTVEGVEALKDAILAALP

SEQ ID NO: 618 - protein, 46 aa, Artificial
  Engineered binding domain
LAQAKCAANAELDSYGVSDFYKRLIDKAKTVEGVEALKDAILAALP

SEQ ID NO: 619 - protein, 46 aa, Artificial
  Engineered binding domain
LAQAKCAANSELDSYGVSDFYKRLIDKAKTVEGVEALKDAILAALP

SEQ ID NO: 620 - protein, 46 aa, Artificial
  Engineered binding domain
LAQAKCSANSELDSYGVSDFYKRLIDKAKTVEGVEALKDAILAALP

SEQ ID NO: 621 - protein, 46 aa, Artificial
  Engineered binding domain
LAQAKCSANAELDSYGVSDFYKRLIDKAKTVEGVEALKDAILAALP

SEQ ID NO: 622 - protein, 46 aa, Artificial
  Engineered binding domain
LAQAKCAANAELDKYGVSDFYKRLIDKAKTVEGVEALKDAILAALP

SEQ ID NO: 623 - protein, 46 aa, Artificial
  Engineered binding domain
LAQAKCAANSELDKYGVSDFYKRLIDKAKTVEGVEALKDAILAALP

SEQ ID NO: 624 - protein, 46 aa, Artificial
  Engineered binding domain
LASAKCAANAELDAYGVSDFYKRLIDKAKTVEGVEALKDAILAALP

SEQ ID NO: 625 - protein, 46 aa, Artificial
  Engineered binding domain
LASAKCAANSELDAYGVSDFYKRLIDKAKTVEGVEALKDAILAALP

SEQ ID NO: 626 - protein, 46 aa, Artificial
  Engineered binding domain
LASAKCSANSELDAYGVSDFYKRLIDKAKTVEGVEALKDAILAALP

SEQ ID NO: 627 - protein, 46 aa, Artificial
  Engineered binding domain
LASAKCSANAELDAYGVSDFYKRLIDKAKTVEGVEALKDAILAALP

SEQ ID NO: 628 - protein, 46 aa, Artificial
  Engineered binding domain
LASAKCAANAELDCYGVSDFYKRLIDKAKTVEGVEALKDAILAALP

SEQ ID NO: 629 - protein, 46 aa, Artificial
  Engineered binding domain
LASAKCAANSELDCYGVSDFYKRLIDKAKTVEGVEALKDAILAALP

SEQ ID NO: 630 - protein, 46 aa, Artificial
  Engineered binding domain
LASAKCAANAELDSYGVSDFYKRLIDKAKTVEGVEALKDAILAALP

SEQ ID NO: 631 - protein, 46 aa, Artificial
  Engineered binding domain
LASAKCAANSELDSYGVSDFYKRLIDKAKTVEGVEALKDAILAALP

SEQ ID NO: 632 - protein, 46 aa, Artificial
  Engineered binding domain
LASAKCSANSELDSYGVSDFYKRLIDKAKTVEGVEALKDAILAALP

SEQ ID NO: 633 - protein, 46 aa, Artificial
  Engineered binding domain
LASAKCSANAELDSYGVSDFYKRLIDKAKTVEGVEALKDAILAALP

SEQ ID NO: 634 - protein, 46 aa, Artificial
  Engineered binding domain
LASAKCAANAELDKYGVSDFYKRLIDKAKTVEGVEALKDAILAALP

SEQ ID NO: 635 - protein, 46 aa, Artificial
  Engineered binding domain
LASAKCAANSELDKYGVSDFYKRLIDKAKTVEGVEALKDAILAALP

SEQ ID NO: 636 - protein, 46 aa, Artificial
  Engineered binding domain
LAEAKCAANAELDAYGVSDFYKRLIDKAKTVEGVEALKDAILAALP

SEQ ID NO: 637 - protein, 46 aa, Artificial
  Engineered binding domain
LAEAKCAANSELDAYGVSDFYKRLIDKAKTVEGVEALKDAILAALP

SEQ ID NO: 638 - protein, 46 aa, Artificial
  Engineered binding domain
LAEAKCSANSELDAYGVSDFYKRLIDKAKTVEGVEALKDAILAALP

SEQ ID NO: 639 - protein, 46 aa, Artificial
  Engineered binding domain
LAEAKCSANAELDAYGVSDFYKRLIDKAKTVEGVEALKDAILAALP

SEQ ID NO: 640 - protein, 46 aa, Artificial
  Engineered binding domain
LAEAKCAANAELDCYGVSDFYKRLIDKAKTVEGVEALKDAILAALP

SEQ ID NO: 641 - protein, 46 aa, Artificial
  Engineered binding domain
LAEAKCAANSELDCYGVSDFYKRLIDKAKTVEGVEALKDAILAALP

SEQ ID NO: 642 - protein, 46 aa, Artificial
  Engineered binding domain
LAEAKCAANAELDSYGVSDFYKRLIDKAKTVEGVEALKDAILAALP

SEQ ID NO: 643 - protein, 46 aa, Artificial
  Engineered binding domain
LAEAKCAANSELDSYGVSDFYKRLIDKAKTVEGVEALKDAILAALP

SEQ ID NO: 644 - protein, 46 aa, Artificial
  Engineered binding domain
LAEAKCSANSELDSYGVSDFYKRLIDKAKTVEGVEALKDAILAALP

SEQ ID NO: 645 - protein, 46 aa, Artificial
  Engineered binding domain
LAEAKCSANAELDSYGVSDFYKRLIDKAKTVEGVEALKDAILAALP

SEQ ID NO: 646 - protein, 46 aa, Artificial
  Engineered binding domain
LAEAKCAANAELDKYGVSDFYKRLIDKAKTVEGVEALKDAILAALP

SEQ ID NO: 647 - protein, 46 aa, Artificial
  Engineered binding domain
LAEAKCAANSELDKYGVSDFYKRLIDKAKTVEGVEALKDAILAALP

SEQ ID NO: 648 - protein, 46 aa, Artificial
  Engineered binding domain
LACAKCAANAELDAYGVSDFYKRLIDKAKTVEGVEALKDAILAALP

SEQ ID NO: 649 - protein, 46 aa, Artificial
  Engineered binding domain
LACAKCAANSELDAYGVSDFYKRLIDKAKTVEGVEALKDAILAALP

SEQ ID NO: 650 - protein, 46 aa, Artificial
  Engineered binding domain
LACAKCSANSELDAYGVSDFYKRLIDKAKTVEGVEALKDAILAALP

SEQ ID NO: 651 - protein, 46 aa, Artificial
  Engineered binding domain
LACAKCSANAELDAYGVSDFYKRLIDKAKTVEGVEALKDAILAALP

SEQ ID NO: 652 - protein, 46 aa, Artificial
  Engineered binding domain
LACAKCAANAELDCYGVSDFYKRLIDKAKTVEGVEALKDAILAALP

SEQ ID NO: 653 - protein, 46 aa, Artificial
  Engineered binding domain
LACAKCAANSELDCYGVSDFYKRLIDKAKTVEGVEALKDAILAALP

SEQ ID NO: 654 - protein, 46 aa, Artificial
  Engineered binding domain
LACAKCAANAELDSYGVSDFYKRLIDKAKTVEGVEALKDAILAALP

SEQ ID NO: 655 - protein, 46 aa, Artificial
  Engineered binding domain
LACAKCAANSELDSYGVSDFYKRLIDKAKTVEGVEALKDAILAALP

SEQ ID NO: 656 - protein, 46 aa, Artificial
  Engineered binding domain
LACAKCSANSELDSYGVSDFYKRLIDKAKTVEGVEALKDAILAALP

SEQ ID NO: 657 - protein, 46 aa, Artificial
  Engineered binding domain
LACAKCSANAELDSYGVSDFYKRLIDKAKTVEGVEALKDAILAALP

SEQ ID NO: 658 - protein, 46 aa, Artificial
  Engineered binding domain
LACAKCAANAELDKYGVSDFYKRLIDKAKTVEGVEALKDAILAALP

SEQ ID NO: 659 - protein, 46 aa, Artificial
  Engineered binding domain
LACAKCAANSELDKYGVSDFYKRLIDKAKTVEGVEALKDAILAALP

SEQ ID NO: 660 - protein, 49 aa, Artificial
  Engineered binding domain
GSLASAKEAANAELDSYGVSDFYKRLIDKAKTVEGVEALKDAILAALPG

SEQ ID NO: 661 - protein, 49 aa, Artificial
  Engineered binding domain
GSLAEAKEAANAELDSYGVSDFYKRLIDKAKTVEGVEALKDAILAALPG

SEQ ID NO: 662 - protein, 49 aa, Artificial
  Engineered binding domain
GSLASAKEAANAELDCYGVSDFYKRLIDKAKTVEGVEALKDAILAALPG

SEQ ID NO: 663 - protein, 49 aa, Artificial
  Engineered binding domain
GSLAEAKEAANAELDCYGVSDFYKRLIDKAKTVEGVEALKDAILAALPG

SEQ ID NO: 664 - protein, 50 aa, Artificial
  Engineered binding domain
GSLASAKEAANAELDSYGVSDFYKRLIDKAKTVEGVEALKDAILAALPCG

SEQ ID NO: 665 - protein, 50 aa, Artificial
  Engineered binding domain
GSLAEAKEAANAELDSYGVSDFYKRLIDKAKTVEGVEALKDAILAALPCG

SEQ ID NO: 666 - protein, 50 aa, Artificial
  Engineered binding domain
GCSLASAKEAANAELDKYGVSDFYKRLIDKAKTVEGVEALKDAILAALPG

SEQ ID NO: 667 - protein, 50 aa, Artificial
  Engineered binding domain
GCSLAEAKEAANAELDKYGVSDFYKRLIDKAKTVEGVEALKDAILAALPG

SEQ ID NO: 668 - protein, 47 aa, Artificial
  Engineered binding domain
GLAEAKVLANRELDKYGVSDYYKNLINNAKTVEGVKALIDEILAALP

SEQ ID NO: 669 - protein, 49 aa, Artificial
  Engineered binding domain
GSSLAEAKVLANRELDKYGVSDFYKRLINKAKTVEGVEALKLHILAALP

SEQ ID NO: 670 - protein, 49 aa, Artificial
  Engineered binding domain
GSSLASAKEAANAELDAYGVSDFYKRLIDKAKTVEGVEALKDAILAALP

SEQ ID NO: 671 - protein, 49 aa, Artificial
  Engineered binding domain
GSSLASAKEAANAELDSYGVSDFYKRLIDKAKTVEGVEALKDAILAALP

SEQ ID NO: 672 - protein, 47 aa, Artificial
  Engineered binding domain
GLASAKEAANAELDSYGVSDFYKRLIDKAKTVEGVEALKDAILAALP

SEQ ID NO: 673 - protein, 47 aa, Artificial
  Engineered binding domain
GLAEAKEAANAELDSYGVSDFYKRLIDKAKTVEGVEALKDAILAALP

SEQ ID NO: 674 - protein, 47 aa, Artificial
  Engineered binding domain
GLASAKEAANAELDCYGVSDFYKRLIDKAKTVEGVEALKDAILAALP

SEQ ID NO: 675 - protein, 47 aa, Artificial
  Engineered binding domain
ALASAKEAANAELDCYGVSDFYKRLIDKAKTVEGVEALKDAILAALP

SEQ ID NO: 676 - protein, 49 aa, Artificial
  Engineered binding domain
GSSLASAKEAANAELDKYGVSDFYKRLIDKAKTVEGVEALKDAILAALP

SEQ ID NO: 677 - protein, 48 aa, Artificial
  Engineered binding domain
GSLASAKEAANAELDSYGVSDFYKRLIDKAKTVEGVEALKDAILAALP

SEQ ID NO: 678 - protein, 48 aa, Artificial
  Engineered binding domain
GSLAEAKEAANAELDSYGVSDFYKRLIDKAKTVEGVEALKDAILAALP

SEQ ID NO: 679 - protein, 46 aa, Artificial
  Engineered binding domain
LAEAKEAANRELDSYGVSDFYKRLIDKAKTVEGVEALKDAILAALP

SEQ ID NO: 680 - protein, 46 aa, Artificial
  Engineered binding domain
LAEAKEAANAELDSYGVSDFYKRLIEKAKTVEGVEALKDAILAALP

SEQ ID NO: 681 - protein, 46 aa, Artificial
  Engineered binding domain
LAEAKEAANAELDSYGVSDFYKRLIDKAKTVEGVEALKEAILAALP

SEQ ID NO: 682 - protein, 46 aa, Artificial
  Engineered binding domain
LAEAKEAANAELDSYGVSDFYKRLIEKAKTVEGVEALKEAILAALP

SEQ ID NO: 683 - protein, 46 aa, Artificial
  Engineered binding domain
LAEAKEAANRELDSYGVSDFYKRLIEKAKTVEGVEALKEAILAALP

SEQ ID NO: 684 - protein, 46 aa, Artificial
  Engineered binding domain
LAEAKEAANAELDSYGVSDFYKRLIDKAKTVEGVEALKDAILKALP

SEQ ID NO: 685 - protein, 46 aa, Artificial
  Engineered binding domain
LAEAKEAANAELDSYGVSDFYKRLIDKAKTVEGVEALKDAILASLP

SEQ ID NO: 686 - protein, 46 aa, Artificial
  Engineered binding domain
LAEAKEAANRELDAYGVSDFYKRLIDKAKTVEGVEALKDAILAALP

SEQ ID NO: 687 - protein, 46 aa, Artificial
  Engineered binding domain
LAEAKEAANAELDAYGVSDFYKRLIEKAKTVEGVEALKDAILAALP

SEQ ID NO: 688 - protein, 46 aa, Artificial
  Engineered binding domain
LAEAKEAANAELDAYGVSDFYKRLIDKAKTVEGVEALKEAILAALP

SEQ ID NO: 689 - protein, 46 aa, Artificial
  Engineered binding domain
LAEAKEAANAELDAYGVSDFYKRLIEKAKTVEGVEALKEAILAALP

SEQ ID NO: 690 - protein, 46 aa, Artificial
  Engineered binding domain
LAEAKEAANRELDAYGVSDFYKRLIEKAKTVEGVEALKEAILAALP

SEQ ID NO: 691 - protein, 46 aa, Artificial
  Engineered binding domain
LAEAKEAANAELDAYGVSDFYKRLIDKAKTVEGVEALKDAILKALP

SEQ ID NO: 692 - protein, 46 aa, Artificial
  Engineered binding domain
LAEAKEAANAELDAYGVSDFYKRLIDKAKTVEGVEALKDAILASLP

SEQ ID NO: 693 - protein, 46 aa, Artificial
  Engineered binding domain
LAQAKEAANRELDAYGVSDFYKRLIDKAKTVEGVEALKDAILAALP

SEQ ID NO: 694 - protein, 46 aa, Artificial
  Engineered binding domain
LAQAKEAANAELDAYGVSDFYKRLIEKAKTVEGVEALKDAILAALP

SEQ ID NO: 695 - protein, 46 aa, Artificial
  Engineered binding domain
LAQAKEAANAELDAYGVSDFYKRLIDKAKTVEGVEALKEAILAALP

SEQ ID NO: 696 - protein, 46 aa, Artificial
  Engineered binding domain
LAQAKEAANAELDAYGVSDFYKRLIEKAKTVEGVEALKEAILAALP

SEQ ID NO: 697 - protein, 46 aa, Artificial
  Engineered binding domain
LAQAKEAANRELDAYGVSDFYKRLIEKAKTVEGVEALKEAILAALP

SEQ ID NO: 698 - protein, 46 aa, Artificial
  Engineered binding domain
LAQAKEAANAELDAYGVSDFYKRLIDKAKTVEGVEALKDAILKALP

SEQ ID NO: 699 - protein, 46 aa, Artificial
  Engineered binding domain
LAQAKEAANAELDAYGVSDFYKRLIDKAKTVEGVEALKDAILASLP

SEQ ID NO: 700 - protein, 46 aa, Artificial
  Engineered binding domain
LAQAKEAANRELDSYGVSDFYKRLIDKAKTVEGVEALKDAILAALP

SEQ ID NO: 701 - protein, 46 aa, Artificial
  Engineered binding domain
LAQAKEAANAELDSYGVSDFYKRLIEKAKTVEGVEALKDAILAALP

SEQ ID NO: 702 - protein, 46 aa, Artificial
  Engineered binding domain
LAQAKEAANAELDSYGVSDFYKRLIDKAKTVEGVEALKEAILAALP

SEQ ID NO: 703 - protein, 46 aa, Artificial
  Engineered binding domain
LAQAKEAANAELDSYGVSDFYKRLIEKAKTVEGVEALKEAILAALP

SEQ ID NO: 704 - protein, 46 aa, Artificial
  Engineered binding domain
LAQAKEAANRELDSYGVSDFYKRLIEKAKTVEGVEALKEAILAALP

SEQ ID NO: 705 - protein, 46 aa, Artificial
  Engineered binding domain
LAQAKEAANAELDSYGVSDFYKRLIDKAKTVEGVEALKDAILKALP

SEQ ID NO: 706 - protein, 46 aa, Artificial
  Engineered binding domain
LAQAKEAANAELDSYGVSDFYKRLIDKAKTVEGVEALKDAILASLP

SEQ ID NO: 707 - protein, 46 aa, Artificial
  Engineered binding domain
LAEAKEAANRELDCYGVSDFYKRLIDKAKTVEGVEALKDAILAALP

SEQ ID NO: 708 - protein, 46 aa, Artificial
  Engineered binding domain
LAEAKEAANAELDCYGVSDFYKRLIEKAKTVEGVEALKDAILAALP

SEQ ID NO: 709 - protein, 46 aa, Artificial
  Engineered binding domain
LAEAKEAANAELDCYGVSDFYKRLIDKAKTVEGVEALKEAILAALP

SEQ ID NO: 710 - protein, 46 aa, Artificial
  Engineered binding domain
LAEAKEAANAELDCYGVSDFYKRLIEKAKTVEGVEALKEAILAALP

SEQ ID NO: 711 - protein, 46 aa, Artificial
  Engineered binding domain
LAEAKEAANRELDCYGVSDFYKRLIEKAKTVEGVEALKEAILAALP

SEQ ID NO: 712 - protein, 46 aa, Artificial
  Engineered binding domain
LAEAKEAANAELDCYGVSDFYKRLIDKAKTVEGVEALKDAILKALP

SEQ ID NO: 713 - protein, 46 aa, Artificial
  Engineered binding domain
LAEAKEAANAELDCYGVSDFYKRLIDKAKTVEGVEALKDAILASLP

SEQ ID NO: 714 - protein, 45 aa, Artificial
  Engineered binding domain
LAEAKEAANAELDSYGVSDFYKRLIDKAKTVEGVEALKDAILAAL

SEQ ID NO: 715 - protein, 45 aa, Artificial
  Engineered binding domain
LAEAKEAANAELDAYGVSDFYKRLIDKAKTVEGVEALKDAILAAL

SEQ ID NO: 716 - protein, 45 aa, Artificial
  Engineered binding domain
LAQAKEAANAELDAYGVSDFYKRLIDKAKTVEGVEALKDAILAAL

SEQ ID NO: 717 - protein, 45 aa, Artificial
  Engineered binding domain
LAQAKEAANAELDSYGVSDFYKRLIDKAKTVEGVEALKDAILAAL

SEQ ID NO: 718 - protein, 45 aa, Artificial
  Engineered binding domain
LAEAKEAANAELDCYGVSDFYKRLIDKAKTVEGVEALKDAILAAL

SEQ ID NO: 719 - protein, 48 aa, Artificial
  Engineered binding domain
EXXXAXXEIXXLPNLTXXQXXAFIXKLXDDPSQSSELLSEAKKLNDSQ

SEQ ID NO: 720 - protein, 53 aa, Artificial
  Engineered binding domain
AKYAKEXXXAXXEIXXLPNLTXXQXXAFIXKLXDDPSQSSELLSEAKKLNDSQ

SEQ ID NO: 721 - protein, 58 aa, Artificial
  Engineered binding domain
VDNKFNKEXXXAXXEIXXLPNLNXXQXXAFIXSLXDDPSQSANLLAEAKKLNDAQAPK

SEQ ID NO: 722 - protein, 26 aa, Artificial Sequence
  Engineered Polypeptide
GVSDXYKXXIXXAXTVEGVXALXXXI

SEQ ID NO: 723 - protein, 20 aa, Artificial Sequence
  Engineered Polypeptide
LAEAKXXAXXELXKYLAALP

SEQ ID NO: 724 - protein, 46 aa, Artificial Sequence
  Engineered Polypeptide
LAXAKXXANXELDXYGVSDFYKRLIXKAKTVEGVEALKXXILXXLP